(19) United States
(12) Patent Application Publication
(10) Pub. No.: US 2026/0035411 A1
(43) Pub. Date: Feb. 5, 2026

(54) CRYSTALLINE FORMS OF A PCSK9 INHIBITOR, COMPOSITIONS AND USES

(71) Applicants: MERCK SHARP & DOHME LLC, Rahway, NJ (US); WERTHENSTEIN BIOPHARMA GMBH, Schachen (CH)

(72) Inventors: Jongrock KONG, Princeton, NJ (US); Richard J. VARSOLONA, Scotch Plains, NJ (US); Richard DESMOND, Lebanon, NJ (US); Peter E. MALIGRES, Rahway, NJ (US); Fuh-Rong TSAY, Rahway, NJ (US); Carlos Alberto Pons SIEPERMANN, Rahway, NJ (US); Artis KLAPARS, Rahway, NJ (US); Lee ROBISON, Rahway, NJ (US); Alfred LEE, Rahway, NJ (US); Tiffany PIOU, Rahway, NJ (US); Lorenzo CODAN, Schachen (CH); Patrick LARPENT, Schachen (CH)

(21) Appl. No.: 19/100,813

(22) PCT Filed: Aug. 16, 2023
(86) PCT No.: PCT/US2023/072326

§ 371 Date: Feb. 3, 2025

Related U.S. Application Data
(60) Provisional application No. 63/371,690, filed on Aug. 17, 2022.
(60) Provisional application No. 63/384,298, filed on Nov. 18, 2022.

(51) Int. Cl. C07K 7/64; A61K 38/00

(52) U.S. Cl. CPC C07K 7/64; A61K 38/00

(57) ABSTRACT

This disclosure provides crystalline forms of a compound of Formula (I), as well as pharmaceutically acceptable compositions thereof, and methods for their preparation and use in methods of treating hypercholesterolemia and other conditions related to PCSK9 activity, e.g., atherosclerosis, atherosclerotic cardiovascular disease, peripheral arterial disease, cerebrovascular disease, coronary heart disease, metabolic syndrome, acute coronary syndrome, or related cardiovascular disease and cardiometabolic conditions.

### RELATED APPLICATIONS

This application claims priority to U.S. Provisional Application No. 63/371,690, filed on Aug. 17, 2022, and U.S. Provisional Application No. 63/384,298, filed on Nov. 18, 2022. The contents of each application are hereby incorporated by reference in their entireties.

### TECHNICAL FIELD

Provided herein are crystalline forms of a compound of Formula I:

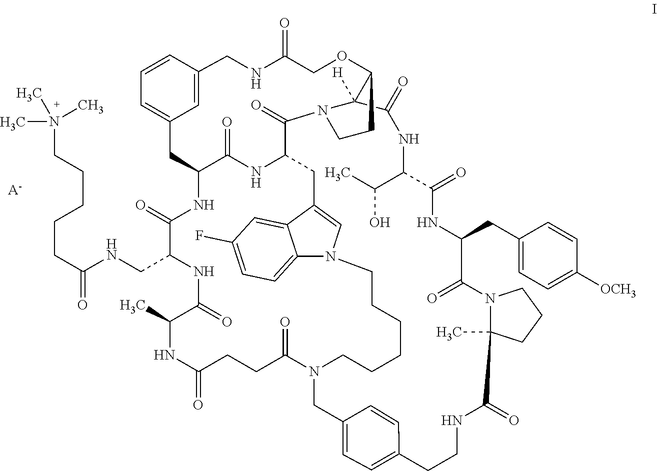

wherein A⁻ is a pharmaceutically acceptable anion, as well as pharmaceutically acceptable compositions thereof, and methods for their preparation and use in methods of treating hypercholesterolemia and other conditions related to PCSK9 activity, e.g., atherosclerosis, atherosclerotic cardiovascular disease, peripheral arterial disease, cerebrovascular disease, coronary heart disease, metabolic syndrome, acute coronary syndrome, or related cardiovascular disease and cardiometabolic conditions.

### BACKGROUND

The solid state of a compound can be important when the compound is used for pharmaceutical purposes. The physical properties of a compound can change from one solid form to another, which can affect the suitability of the form for pharmaceutical use. For example, a particular crystalline solid compound can overcome the disadvantage of other solid forms of the compound such as, e.g., instability and/or reduced purity.

Provided herein are crystalline forms of a compound of Formula I:

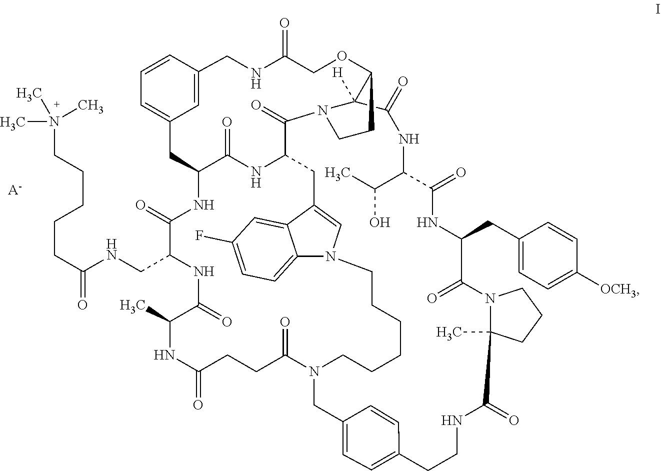

wherein A⁻ is a pharmaceutically acceptable anion. These crystalline forms of a compound of Formula I enable efficient isolation and purification, avoiding the need for expensive operations such as chromatography and lyophilization. In addition, the crystalline forms of a compound of Formula I are advantageous in that they have high purity, high stability, and lower hygroscopicity, making them suitable for use in pharmaceutical formulations.

### SUMMARY

The disclosure relates to a crystalline form of a compound of Formula I:

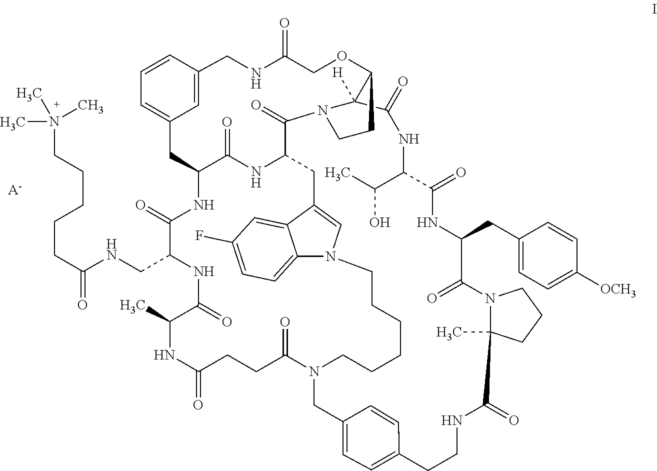

wherein A⁻ is a pharmaceutically acceptable anion, which has activity as a PCSK9 inhibitor, as well as compositions comprising the same, methods of making the same, and methods of using the same.

### BRIEF DESCRIPTION OF THE DRAWINGS

FIG. 1 depicts an X-ray powder diffraction pattern of Amorphous Acetate 1, showing a range of 2-40 2θ. The graph plots the intensity of the peaks as defined by counts per second versus the diffraction angle 2 theta (2θ) in degrees.

FIG. 2 depicts an X-ray powder diffraction pattern of Acetate 2, showing a range of 2-40 2θ. The graph plots the intensity of the peaks as defined by counts per second versus the diffraction angle 2 theta (2θ) in degrees.

FIG. 3 depicts an X-ray powder diffraction pattern of Acetate 3, showing a range of 2-40 2θ. The graph plots the intensity of the peaks as defined by counts per second versus the diffraction angle 2 theta (2θ) in degrees.

FIG. 4 depicts an X-ray powder diffraction pattern of Acetate 4, showing a range of 2-40 2θ. The graph plots the intensity of the peaks as defined by counts per second versus the diffraction angle 2 theta (2θ) in degrees.

FIG. 5 depicts an X-ray powder diffraction pattern of Acetate 5, showing a range of 2-40 2θ. The graph plots the intensity of the peaks as defined by counts per second versus the diffraction angle 2 theta (2θ) in degrees.

FIG. 6 depicts an X-ray powder diffraction pattern of Acetate 6, showing a range of 2-40 2θ. The graph plots the intensity of the peaks as defined by counts per second versus the diffraction angle 2 theta (2θ) in degrees.

FIG. 7 depicts an X-ray powder diffraction pattern of Amorphous Caprate 1, showing a range of 2-40 2θ. The graph plots the intensity of the peaks as defined by counts per second versus the diffraction angle 2 theta (2θ) in degrees.

FIG. 8 depicts an X-ray powder diffraction pattern of Caprate 2, showing a range of 2-40 2θ. The graph plots the intensity of the peaks as defined by counts per second versus the diffraction angle 2 theta (2θ) in degrees.

FIG. 9 depicts an X-ray powder diffraction pattern of Caprate 3, showing a range of 2-40 2θ. The graph plots the intensity of the peaks as defined by counts per second versus the diffraction angle 2 theta (2θ) in degrees.

FIG. 10 depicts an X-ray powder diffraction pattern of Caprate 4, showing a range of 2-40 2θ. The graph plots the intensity of the peaks as defined by counts per second versus the diffraction angle 2 theta (2θ) in degrees.

FIG. 11 depicts an X-ray powder diffraction pattern of Caprate 5, showing a range of 2-40 2θ. The graph plots the intensity of the peaks as defined by counts per second versus the diffraction angle 2 theta (2θ) in degrees.

FIG. 12 depicts an X-ray powder diffraction pattern of Caprate 6, showing a range of 2-40 2θ. The graph plots the intensity of the peaks as defined by counts per second versus the diffraction angle 2 theta (2θ) in degrees.

FIG. 13 depicts an X-ray powder diffraction pattern of Caprate 7, showing a range of 2-40 2θ. The graph plots the intensity of the peaks as defined by counts per second versus the diffraction angle 2 theta (2θ) in degrees.

FIG. 14 depicts an X-ray powder diffraction pattern of Caprate 8, showing a range of 2-40 2θ. The graph plots the intensity of the peaks as defined by counts per second versus the diffraction angle 2 theta (2θ) in degrees.

FIG. 15 depicts an X-ray powder diffraction pattern of Caprate 9, showing a range of 2-40 2θ. The graph plots the intensity of the peaks as defined by counts per second versus the diffraction angle 2 theta (2θ) in degrees.

FIG. 16 depicts an X-ray powder diffraction pattern of Caprate 10, showing a range of 2-40 2θ. The graph plots the intensity of the peaks as defined by counts per second versus the diffraction angle 2 theta (2θ) in degrees.

FIG. 17 depicts an X-ray powder diffraction pattern of Caprate 11, showing a range of 2-40 2θ. The graph plots the intensity of the peaks as defined by counts per second versus the diffraction angle 2 theta (2θ) in degrees.

FIG. 18 depicts an X-ray powder diffraction pattern of Caprate 12, showing a range of 2-40 2θ. The graph plots the intensity of the peaks as defined by counts per second versus the diffraction angle 2 theta (2θ) in degrees.

FIG. 19 depicts an X-ray powder diffraction pattern of Caprate 13, showing a range of 2-40 2θ. The graph plots the intensity of the peaks as defined by counts per second versus the diffraction angle 2 theta (2θ) in degrees.

FIG. 20 depicts an X-ray powder diffraction pattern of Caprate 14, showing a range of 2-40 2θ. The graph plots the intensity of the peaks as defined by counts per second versus the diffraction angle 2 theta (2θ) in degrees.

FIG. 21 depicts an X-ray powder diffraction pattern of D-Lactate 1, showing a range of 2-40 2θ. The graph plots the intensity of the peaks as defined by counts per second versus the diffraction angle 2 theta (2θ) in degrees.

FIG. 22 depicts an X-ray powder diffraction pattern of D-Lactate 2, showing a range of 2-40 2θ. The graph plots the intensity of the peaks as defined by counts per second versus the diffraction angle 2 theta (2θ) in degrees.

FIG. 23 depicts an X-ray powder diffraction pattern of Succinate 1, showing a range of 2-40 2θ. The graph plots the intensity of the peaks as defined by counts per second versus the diffraction angle 2 theta (2θ) in degrees.

FIG. 24 depicts an X-ray powder diffraction pattern of Succinate 2, showing a range of 2-40 2θ. The graph plots the intensity of the peaks as defined by counts per second versus the diffraction angle 2 theta (2θ) in degrees.

FIG. 25 depicts an X-ray powder diffraction pattern of L-Tartrate 1, showing a range of 2-40 2θ. The graph plots the intensity of the peaks as defined by counts per second versus the diffraction angle 2 theta (2θ) in degrees.

FIG. 26 depicts an X-ray powder diffraction pattern of L-Tartrate 2, showing a range of 2-40 2θ. The graph plots the intensity of the peaks as defined by counts per second versus the diffraction angle 2 theta (2θ) in degrees.

FIG. 27 depicts an X-ray powder diffraction pattern of Sulfate 1, showing a range of 2-40 2θ. The graph plots the intensity of the peaks as defined by counts per second versus the diffraction angle 2 theta (2θ) in degrees.

FIG. 28 depicts an X-ray powder diffraction pattern of Sulfate 2, showing a range of 2-40 2θ. The graph plots the intensity of the peaks as defined by counts per second versus the diffraction angle 2 theta (2θ) in degrees.

FIG. 29A depicts the percentage of total impurities for Compound A (API Chloride Amorphous; the amorphous form of the chloride salt), Acetate 1, Caprate 1, Caprate 3, and Caprate 7, showing a range of 3 months.

FIG. 29B depicts the percentage of total impurities for Acetate 1 (amorphous acetate salt), Caprate 1 (amorphous caprate salt), Caprate 3, and Caprate 7, showing a range of 3 months.

FIG. 30A depicts the adsorption/desorption cycles of Acetate 4, showing a range of 5-55% relative humidity (RH).

FIG. 30B depicts the X-ray powder diffraction pattern of Acetate 4 before and after the adsorption/desorption cycles of FIG. 30A.

FIG. 31A depicts the adsorption/desorption cycle of Acetate 4, showing a range of 5-95% RH.

FIG. 31B depicts the X-ray powder diffraction pattern of Acetate 4 before and after the adsorption/desorption cycle of FIG. 31A.

FIG. 32A depicts the adsorption/desorption cycles of Caprate 5, showing a range of 5-65% RH.

FIG. 32B depicts the X-ray powder diffraction pattern of Caprate 5 before and after the adsorption/desorption cycles of FIG. 32A.

FIG. 33A depicts the adsorption/desorption cycle of Caprate 5, showing a range of 5-95% RH.

FIG. 33B depicts the X-ray powder diffraction pattern of Caprate 5 before and after the adsorption/desorption cycle of FIG. 33A.

FIG. 34A depicts the adsorption/desorption cycle of Caprate 3, showing a range of 5-85% RH.

FIG. 34B depicts the X-ray powder diffraction pattern of Caprate 3 before and after the adsorption/desorption cycle of FIG. 34A.

FIG. 35A depicts the adsorption/desorption cycle of water-free Caprate 3, showing a range of 5-85% RH.

FIG. 35B depicts the X-ray powder diffraction pattern of water-free Caprate 3 before and after the adsorption/desorption cycle of FIG. 35A.

FIG. 36A depicts the solid-state C-13 CPMAS NMR spectrum for Caprate 3.

FIG. 36B depicts the solid-state C-13 CPMAS NMR spectrum for Caprate 5.

FIG. 36C depicts the solid-state C-13 CPMAS NMR spectrum for Caprate 8.

FIG. 37 depicts selected spectral regions from the caprate C-13 CPMAS spectra of the caprate forms exhibiting form-distinctive features. Spectral regions for Caprate 8, Caprate 5, and Caprate 3 are shown from top to bottom, respectively. Relevant isotropic chemical shifts are given in ppm (parts per million).

### DETAILED DESCRIPTION

Proprotein convertase subtilisin-kexin type 9 (hereinafter called “PCSK9”), also known as neural apoptosis-regulated convertase 1 (“NARC-1”), is a proteinase K-like subtilase identified as the ninth member of the secretory subtilase family; see Seidah et al., 2003 PNAS 100:928-933. PCSK9 belongs to the mammalian proprotein convertase family of serine proteases and contains an N-terminal signal sequence, a prodomain, a catalytic domain, and a C-terminal domain; see Seidah et al., 2012 Nat. Rev. Drug Discov. 11:367-383. A study of PCSK9 transcriptional regulation demonstrated that it is regulated by sterol regulatory element-binding proteins (“SREBP”), as seen with other genes involved in cholesterol metabolism; Maxwell et al., 2003 J. Lipid Res. 44:2109-2119, as is typical of other genes implicated in lipoprotein metabolism; Dubuc et al., 2004 Arterioscler. Thromb. Vasc. Biol. 24:1454-1459. Statins have been shown to upregulate PCSK9 expression in a manner attributed to the cholesterol-lowering effects of the drugs; supra. Moreover, it has been shown that PCSK9 promoters possess two conserved sites involved in cholesterol regulation, a sterol regulatory element and an Sp1 site; supra.

While in the endoplasmic reticulum, PCSK9 performs as its only catalytic activity an autocleavage between residues Gln-152 and Ser-153; see Naureckiene et al., 2003 Arch. Biochem. Biophys. 420:55-67; Seidah et al., 2003 Proc. Natl. Acad. Sci. U.S.A 100:928-933. The prodomain remains tightly associated with the catalytic domain during subsequent trafficking through the trans-Golgi network. The maturation via autocleavage has been demonstrated to be critical for PCSK9 secretion and subsequent extracellular function (see Benjannet et al., 2012 J. Biol. Chem. 287:33745-33755). Accordingly, several lines of evidence demonstrate that PCSK9, in particular, lowers the amount of hepatic LDLR protein and thus compromises the liver's ability to remove low density lipoprotein (“LDL”) cholesterol from the circulation.

Adenovirus-mediated overexpression of PCSK9 in the liver of mice results in the accumulation of circulating low density lipoprotein cholesterol (“LDL-C”) due to a dramatic loss of hepatic LDLR protein, with no effect on LDLR mRNA levels; Benjannet et al., 2004 J. Biol. Chem. 279:48865-48875; Maxwell & Breslow, 2004 PNAS 101:7100-7105; Park et al., 2004 J. Biol. Chem. 279:50630-50638; and Lalanne et al., 2005 J. Lipid Res. 46:1312-1319. The effect of PCSK9 overexpression on raising circulating LDL-C levels in mice is completely dependent on the expression of LDLR, again indicating that the regulation of LDL-C by PCSK9 is mediated through downregulation of LDLR protein. In agreement with these findings, mice lacking PCSK9 or in which PCSK9 mRNA has been lowered by antisense oligonucleotide inhibitors have higher levels of hepatic LDLR protein and a greater ability to clear circulating LDL-C; Rashid et al., 2005 PNAS 102:5374-5379; and Graham et al., 2007 J. Lipid Res. 48 (4): 763-767. In addition, lowering PCSK9 levels in cultured human hepatocytes by siRNA also results in higher LDLR protein levels and an increased ability to take up LDL-C; Benjannet et al., 2004 J. Biol. Chem. 279:48865-48875; and Lalanne et al., 2005 J. Lipid Res. 46:1312-1319. Together, these data indicate that PCSK9 action leads to increased LDL-C by lowering LDLR protein levels.

A number of mutations in the gene PCSK9 have also been conclusively associated with autosomal dominant hypercholesterolemia (“ADH”), an inherited metabolism disorder characterized by marked elevations of low-density lipoprotein (“LDL”) particles in the plasma which can lead to premature cardiovascular failure; see Abifadel et al., 2003 Nature Genetics 34:154-156; Timms et al., 2004 Hum. Genet. 114:349-353; Leren, 2004 Clin. Genet. 65:419-422. A later-published study on the S127R mutation of Abifadel et al., supra, reported that patients carrying such a mutation exhibited higher total cholesterol and apoB 100 in the plasma attributed to (1) an overproduction of apoB 100-containing lipoproteins, such as low density lipoprotein (“LDL”), very low density lipoprotein (“VLDL”) and intermediate density lipoprotein (“IDL”), and (2) an associated reduction in clearance or conversion of said lipoproteins; Ouguerram et al., 2004 Arterioscler. Thromb. Vasc. Biol. 24:1448-1453.

Accordingly, there can be no doubt that PCSK9 plays a role in the regulation of LDL. The expression or upregulation of PCSK9 is associated with increased plasma levels of LDL cholesterol, and the corresponding inhibition or lack of expression of PCSK9 is associated with reduced LDL cholesterol plasma levels. Decreased levels of LDL cholesterol associated with sequence variations in PCSK9 have been found to confer protection against coronary heart disease; Cohen, 2006 N. Engl. J. Med. 354:1264-1272.

In clinical trials, reductions in LDL cholesterol levels have been directly related to the reduction in the rate of coronary events; Law et al., 2003 BMJ 326:1423-1427. The moderate lifelong reduction in plasma LDL cholesterol levels was found to correlate with a substantial reduction in the incidence of coronary events; Cohen et al., 2006 N. Engl. J. Med. 354:1264-1272. This was the case even in populations with a high prevalence of non-lipid-related cardiovascular risk factors; supra. Accordingly, there is great benefit to be reaped from the managed control of LDL cholesterol levels.

Thus, identification of compounds and/or agents effective in the treatment of cardiovascular affliction is highly desirable, including antagonism of PCSK9's role in LDL regulation; however, in general, because PCSK9 circulates in blood and has modest binding affinity to cell surface LDL receptors heretofore attempts to utilize this mechanism in treatment of diseases related to high serum LDL levels have been focused on the use of large biomolecules, for example, antibodies. The therapeutic potential of small peptides or small molecules as drugs targeting PCSK9 has only just begun to be explored; see for example, Tombling et al., Atherosclerosis 330 (2021) 52-60. Moreover, there is a paucity of compounds which are amenable to formulation into a dosage form for utilizing an oral administration route of dosing such compounds, a route which would be highly desirable for the provision of therapy for conditions in which regulation of the activities of PCSK9 could play a role.

WO 2019/246349 discloses cyclic peptide compounds useful in the treatment of cardiovascular disease and conditions related to PCSK9 activity. The present disclosure advances the state of the art by providing crystalline forms of the compound of Formula I, which can be used to treat hypercholesterolemia and other conditions related to PCSK9 activity desirably comprising oral administration of an identified PCSK9 inhibitor. Certain crystalline forms have advantages, such as ease of processing or handling. In particular, these forms may exhibit improved physicochemical properties, such as rendering them particularly suitable for the manufacture of various pharmaceutical dosage forms, including oral dosage forms.

Thus, provided herein are crystalline forms of a salt of a compound of Formula I:

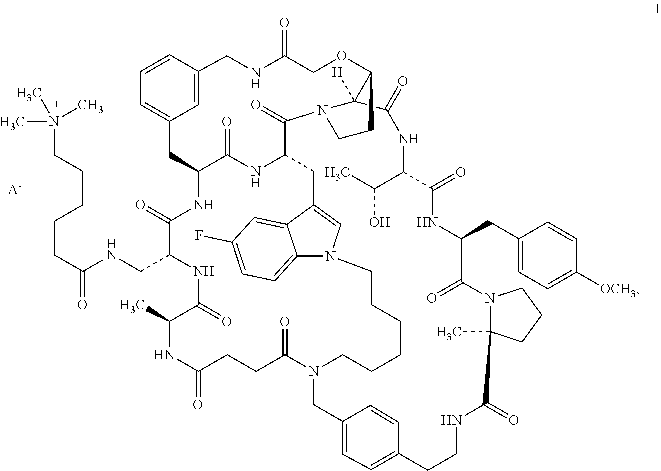

wherein A⁻ is a pharmaceutically acceptable anion. In a further embodiment, A⁻ is selected from acetate, caprate, lactate, tartrate, succinate, and sulfate. The term “caprate” is also known in the art as “decanoate” and may be used interchangeably. In still another embodiment, provided herein is a crystalline form of a compound of Formula I, wherein the crystalline form is selected from Acetate 2, Acetate 3, Acetate 4, Acetate 5, Acetate 6, Caprate 2, Caprate 3, Caprate 4, Caprate 5, Caprate 6, Caprate 7, Caprate 8, Caprate 9, Caprate 10, Caprate 11, Caprate 12, Caprate 13, Caprate 14, D-Lactate 1, D-Lactate 2, Succinate 1, Succinate 2, L-Tartrate 1, L-Tartrate 2, Sulfate 1, and Sulfate 2. Additional aspects of this embodiment of the present disclosure provide a particular drug substance that comprises at least one of the forms described herein. The presence of a particular crystalline form in a drug substance can be detected by physical methods known to those of ordinary skill in the art, such as X-ray powder diffraction (XRPD), single crystal X-ray diffraction, nuclear magnetic resonance (NMR) spectroscopy, or nitrogen-15 CPMAS NMR spectroscopy.

Compound A is the amorphous form of the chloride salt of a compound of Formula I.

Compound B is the bicarbonate salt of a compound of Formula I.

Described herein is an acetate salt of a compound of Formula I, as shown below, and referred to as Compound 1:

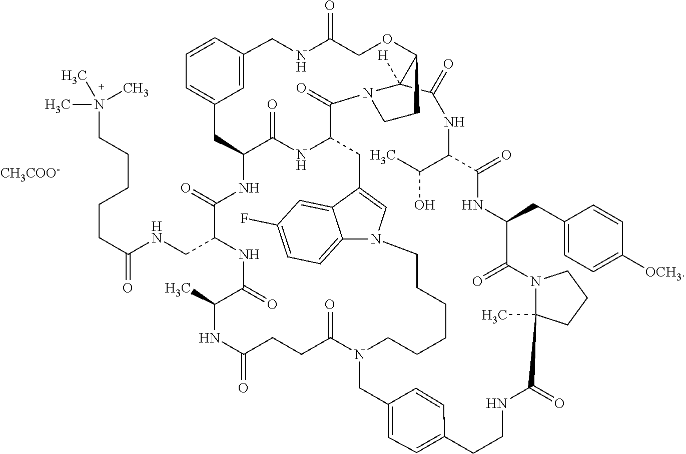

Acetate 1 is the amorphous form of Compound 1.

In an embodiment, provided herein are crystalline forms of Compound 1.

In a further embodiment, provided herein is the crystalline form of Compound 1, wherein the crystalline form is selected from Acetate 2, Acetate 3, Acetate 4, Acetate 5, and Acetate 6.

Also described herein is a caprate salt of a compound of Formula I as seen below and referred to as Compound 2:

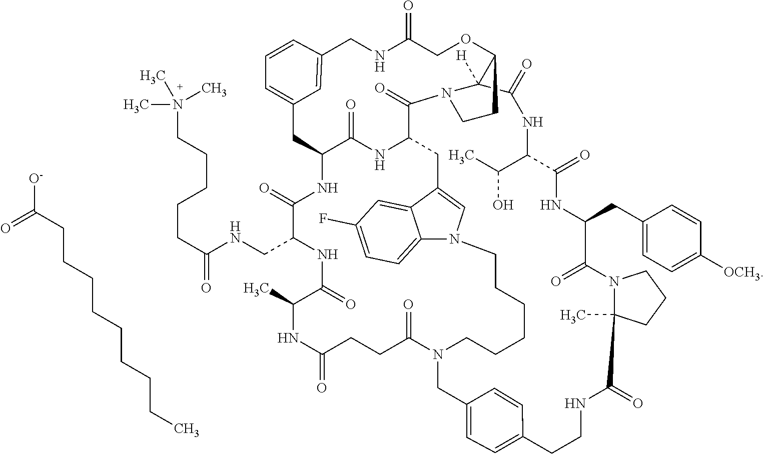

Caprate 1 is the amorphous form of Compound 2.

In an embodiment, provided herein are crystalline forms of Compound 2.

In a further embodiment, provided herein is the crystalline form of Compound 2, wherein the crystalline form is selected from Caprate 2, Caprate 3, Caprate 4, Caprate 5, Caprate 6, Caprate 7, Caprate 8, Caprate 9, Caprate 10, Caprate 11, Caprate 12, Caprate 13, and Caprate 14.

Also described herein is a lactate salt of a compound of Formula I as seen below and referred to as Compound 3:

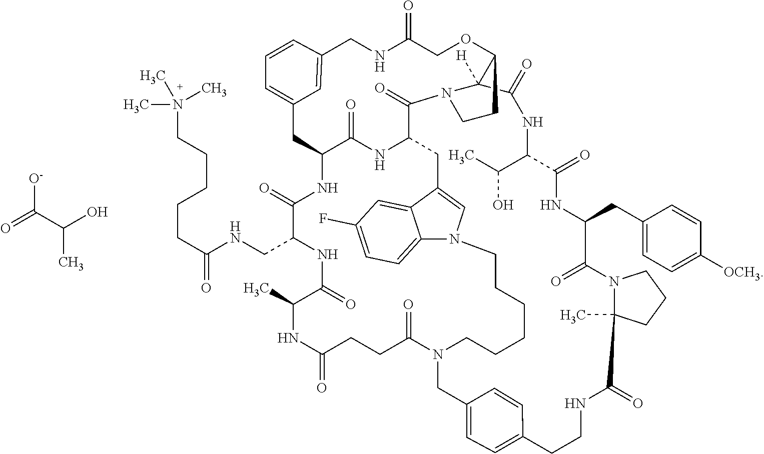

In an embodiment, provided herein are crystalline forms of Compound 3.

In a further embodiment, provided herein is the crystalline form of Compound 3, wherein the crystalline form is selected from D-Lactate 1 and D-Lactate 2. The structure of D-Lactate is depicted below:

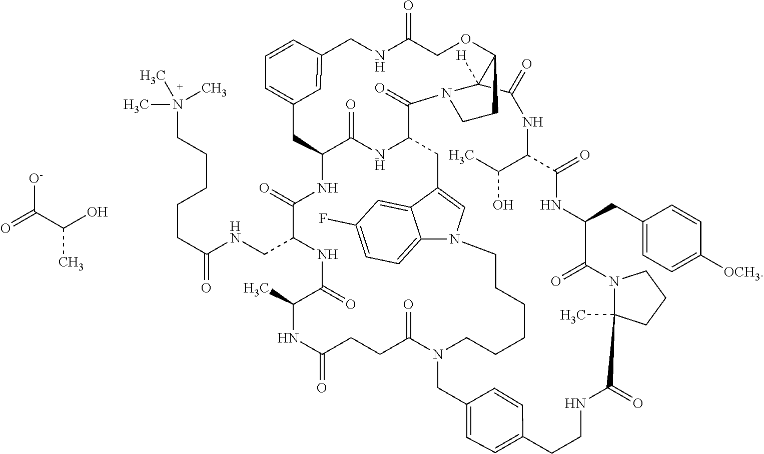

Also described herein is a succinate salt of a compound of Formula I as seen below and referred to as Compound 4:

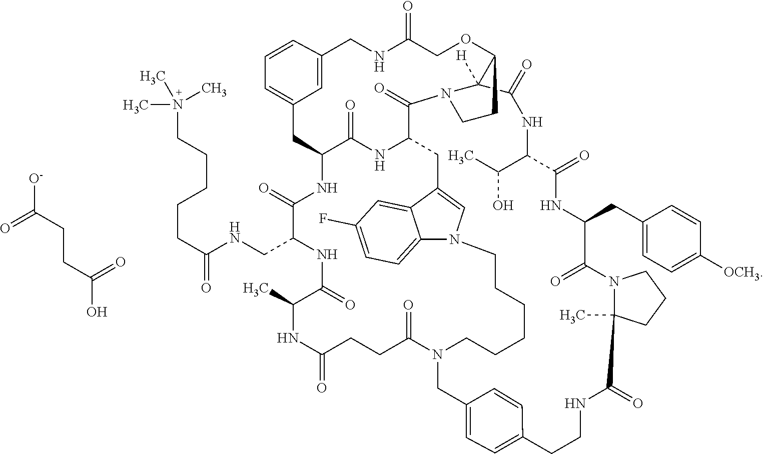

In an embodiment, provided herein are crystalline forms of Compound 4.

In a further embodiment, provided herein is the crystalline form of Compound 4, wherein the crystalline form is selected from Succinate 1 and Succinate 2.

Also described herein is a tartrate salt of a compound of Formula I as seen below and referred to as Compound 5:

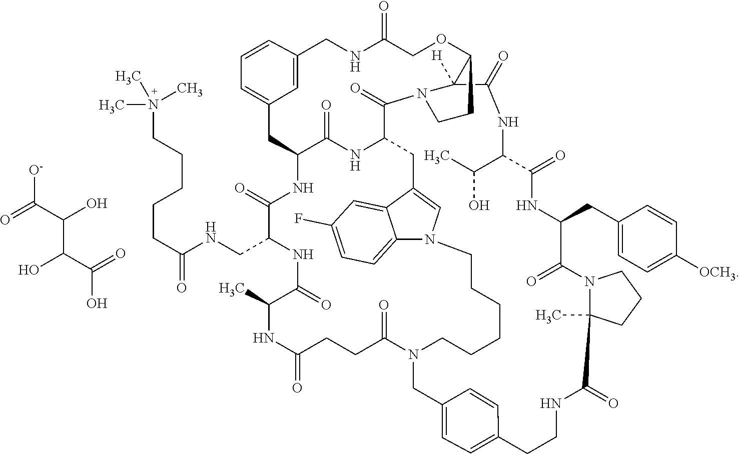

In an embodiment, provided herein are crystalline forms of Compound 5.

In a further embodiment, provided herein is the crystalline form of Compound 5, wherein the crystalline form is selected from L-Tartrate 1 and L-Tartrate 2. The structure of L-Tartrate is depicted below:

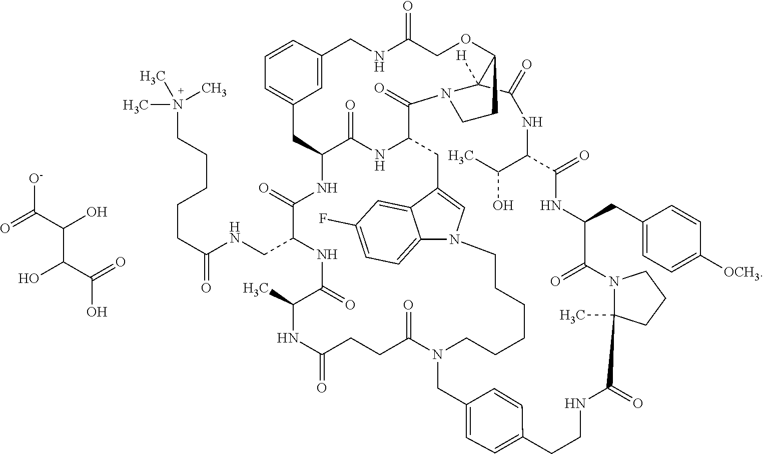

L-Tartrate may also be referred to as (2R,3R)-hydrogen tartrate.

Also described herein is a sulfate salt of a compound of Formula I as seen below and referred to as Compound 6:

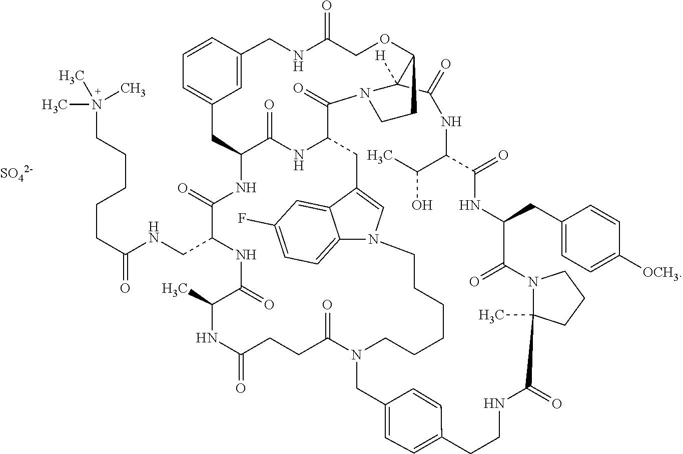

In an embodiment, provided herein are crystalline forms of Compound 6.

In a further embodiment, provided herein is the crystalline form of Compound 6, wherein the crystalline form is selected from Sulfate 1 and Sulfate 2.

In an embodiment of the above structure, the compound of Formula I and the sulfate anion have a stoichiometry of 2:1, as depicted below:

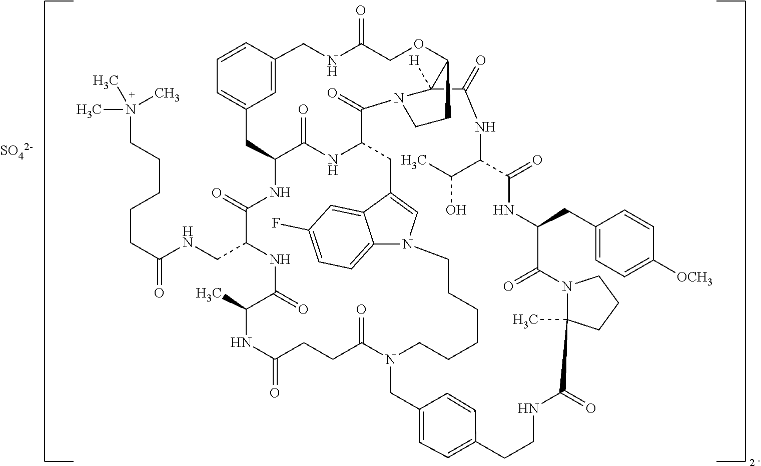

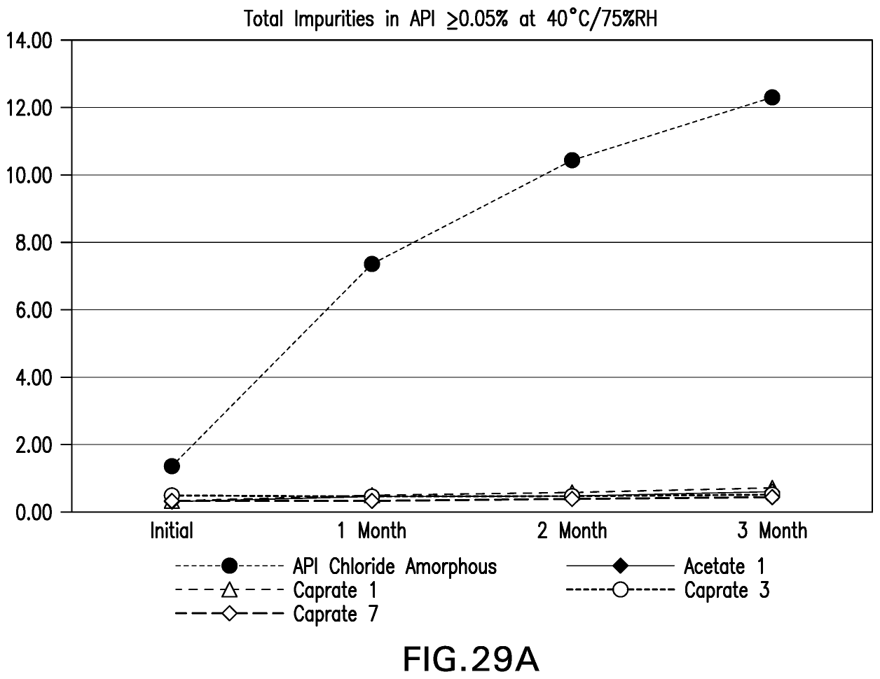

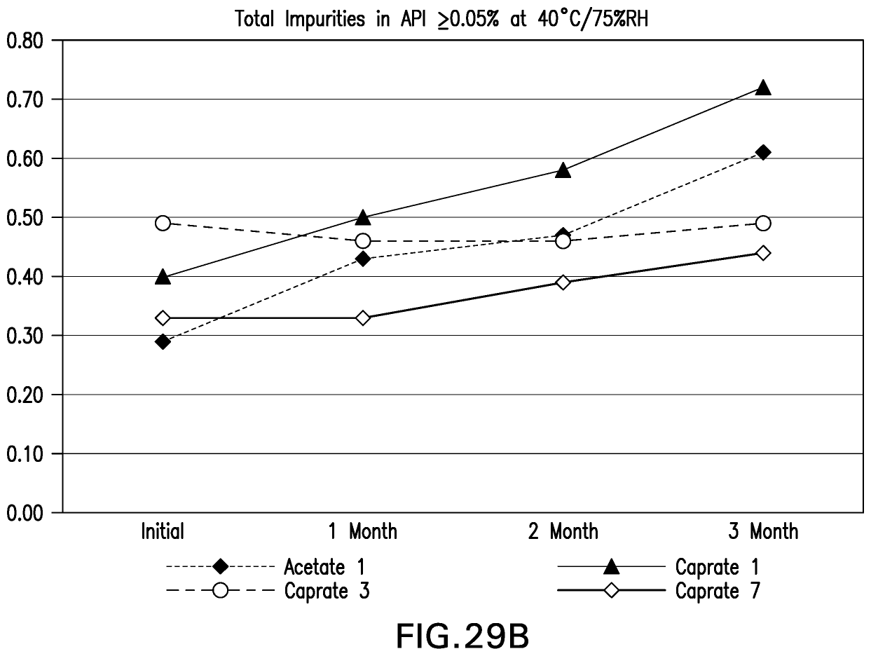

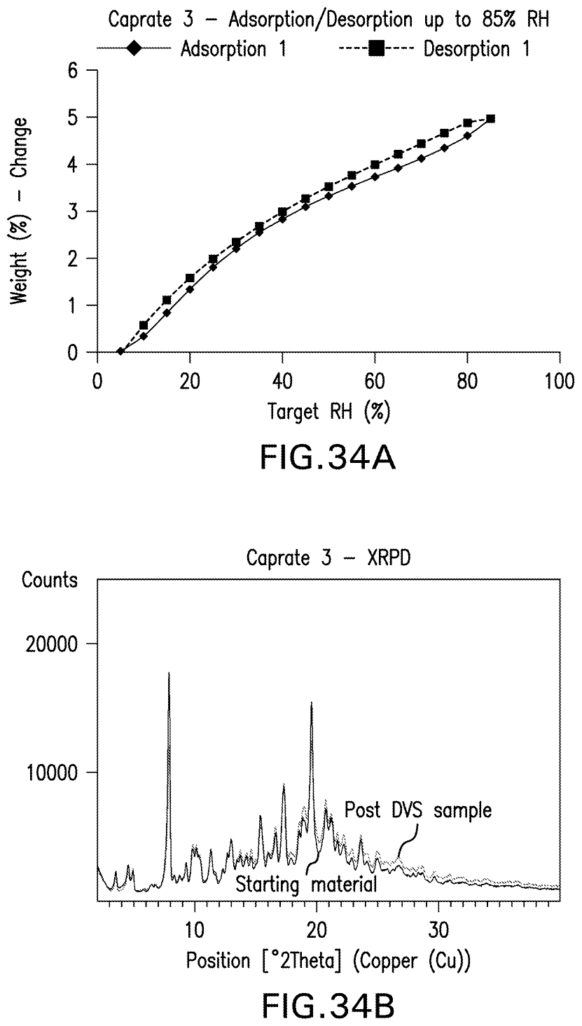

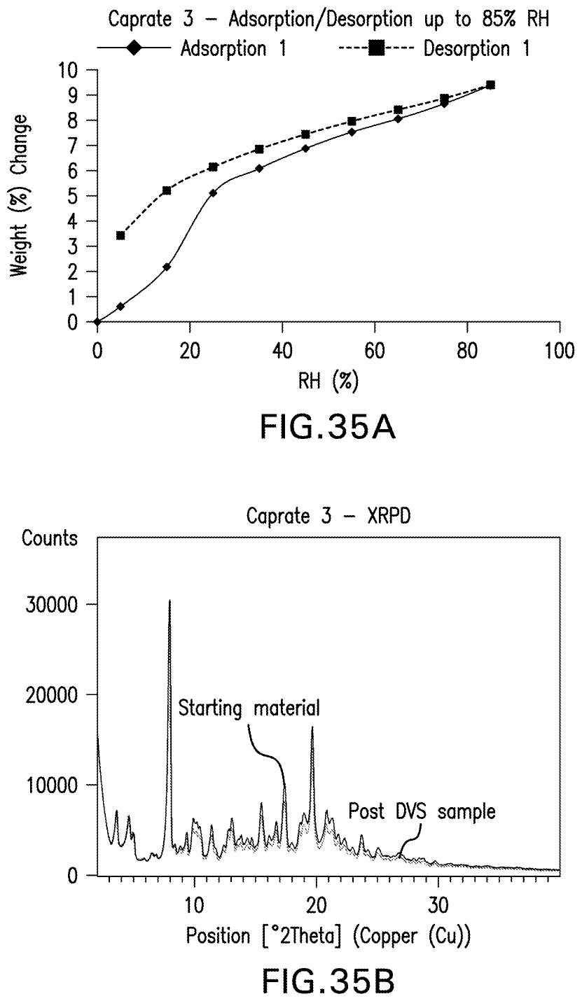

Particular crystalline forms of the compound of Formula I provided herein have advantageous characteristics that are beneficial to the preparation of various drug formulations. For example, a particular crystalline form of the compound of Formula I, Caprate 3, is a stable crystalline form. Caprate 3 maintains its crystallinity (i.e., is physically stable) when subjected to varying relative humidity (see, e.g., FIG. 34A-35B). Crystalline forms with good stability are important in the processes of preparation, packing, transportation, and storage of pharmaceutical products. The processes of making Caprate 3 (see, e.g., Examples 12A, 12B, 12C, and 19) also results in enhanced chemical stability over the amorphous chloride salt, an important feature for preparing and using pharmaceutical products (see, e.g., FIG. 29A and FIG. 29B).

### Characterization of Crystalline Forms

In certain embodiments, the crystalline forms provided herein are identifiable on the basis of characteristic peaks in an X-ray powder diffraction analysis. X-ray powder diffraction (XRPD) is a scientific technique using X-ray diffraction on powder, microcrystalline, or other solid materials for structural characterization of solid materials. A description of the methods used to obtain certain XRPD patterns in connection with the crystalline forms of the invention can be found in Example 34, Description of Powder X-Ray Diffraction. In an embodiment, the X-ray powder diffraction data provided herein is obtained by a method utilizing Cu Kα radiation.

### Crystalline Forms of Compounds of Formula I

### Acetate 2:

In an embodiment, provided herein is Acetate 2, which is a crystalline form of the acetate salt of a compound of Formula I, characterized by an X-ray powder diffraction pattern having peaks expressed in degrees-2-theta at angles of (±0.2°) 4.92, 6.59, 9.82, and 17.91. In particular aspects, Acetate 2 is characterized by an X-ray powder diffraction pattern having peaks expressed in degrees-2-theta at angles of (±0.2°) 4.92, 6.59, 9.82, 16.14, 17.37, 17.91, 19.01, 19.67, and 20.16. In another embodiment, the crystalline form of the compound of Formula I is Acetate 2, wherein the crystalline form is characterized by an X-ray powder diffraction pattern having peaks shown in Table I (expressed in degrees-2-theta at angles ±0.2°).

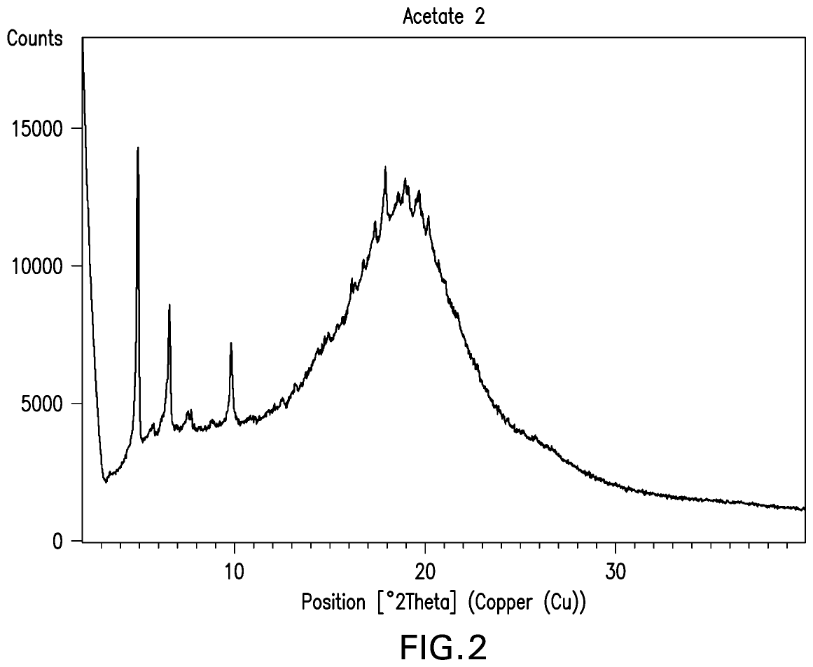

In aspects of this embodiment, about 10% to about 100% of the compound of Formula I in a pharmaceutical composition is in the form of Acetate 2, such as from about 25% to about 98%, from about 50% to about 96%, from about 75% to about 95%, from about 90% to about 94%, or about 92%. In aspects of this embodiment, Acetate 2 is characterized by an X-ray powder diffraction pattern substantially as shown in FIG. 2. In aspects of this embodiment, Acetate 2 is characterized by X-ray powder diffraction substantially as described by one or more of the characteristics recited in Table 1.

TABLE 1
X-Ray powder diffraction pattern of Acetate 2

| 2-θ ° | d-spacing, Å | Relative Intensity, % |
|---|---|---|
| 4.92 | 17.95 | 100 |
| 5.72 | 15.44 | 11 |
| 6.59 | 13.41 | 45 |
| 7.53 | 11.74 | 10 |
| 7.73 | 11.43 | 10 |
| 8.83 | 10.01 | 5 |
| 9.82 | 9.01 | 27 |
| 14.29 | 6.20 | 10 |
| 14.86 | 5.96 | 13 |
| 16.14 | 5.49 | 28 |
| 17.37 | 5.11 | 43 |
| 17.91 | 4.95 | 59 |
| 19.01 | 4.67 | 49 |
| 19.67 | 4.51 | 46 |
| 20.16 | 4.40 | 36 |

### Acetate 3:

In another embodiment, provided herein is Acetate 3, which is a crystalline form of the acetate salt of a compound of Formula I, characterized by an X-ray powder diffraction pattern having peaks expressed in degrees-2-theta at angles (±0.2°) of 4.48, 18.17, 18.79, and 19.27. In particular aspects, Acetate 3 is characterized by an X-ray powder diffraction pattern having peaks expressed in degrees-2-theta at angles (±0.2°) of 4.48, 16.54, 18.17, 18.79, 19.27, 20.64, 20.93, 21.51, 22.18, and 22.65. In still more particular aspects, Acetate 3 is characterized by an X-ray powder diffraction pattern having peaks expressed in degrees-2-theta at angles (±0.2°) of 4.48, 8.97, 9.08, 13.80, 14.51, 16.12, 16.54, 18.17, 18.79, 19.27, 20.64, 20.93, 21.51, 22.18, 22.65, 23.83, 24.29, and 24.57. In another embodiment, the crystalline form of the compound of Formula I is Acetate 3, wherein the crystalline form is characterized by an X-ray powder diffraction pattern having peaks shown in Table 2 (expressed in degrees-2-theta at angles)+0.2°.

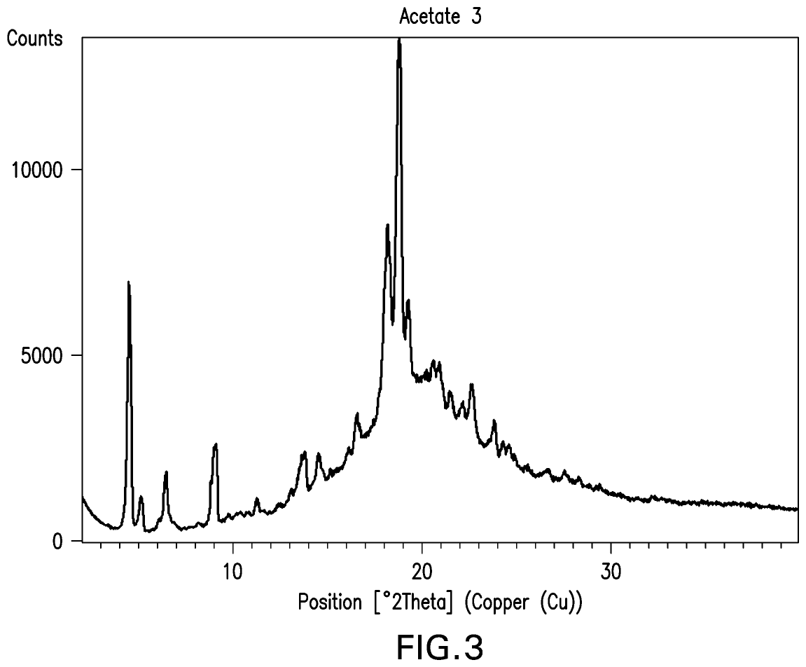

In aspects of this embodiment, about 10% to about 100% of the compound of Formula I in a pharmaceutical composition is in the form of Acetate 3, such as from about 25% to about 98%, from about 50% to about 96%, from about 75% to about 95%, from about 90% to about 94%, or about 92%. In aspects of this embodiment, Acetate 3 is characterized by an X-ray powder diffraction pattern substantially as shown in FIG. 3. In aspects of this embodiment, Acetate 3 is characterized by X-ray powder diffraction substantially as described by one or more of the characteristics recited in Table 2.

TABLE 2
X-Ray powder diffraction pattern of Acetate 3

| | | Relative |
|---|---|---|
| 2-θ ° | d-spacing, Å | Intensity, % |
| 4.48 | 19.72 | 53 |
| 5.11 | 17.30 | 7 |
| 6.06 | 14.58 | 2 |
| 6.44 | 13.72 | 12 |
| 6.96 | 12.70 | 1 |
| 8.15 | 10.85 | 1 |
| 8.81 | 10.03 | 10 |
| 8.97 | 9.86 | 17 |
| 9.08 | 9.73 | 15 |
| 9.78 | 9.04 | 2 |
| 10.39 | 8.51 | 3 |
| 10.78 | 8.20 | 3 |
| 11.29 | 7.84 | 6 |
| 12.42 | 7.13 | 4 |
| 13.07 | 6.77 | 7 |
| 13.80 | 6.42 | 15 |
| 14.51 | 6.10 | 15 |
| 15.15 | 5.85 | 11 |
| 16.12 | 5.50 | 16 |
| 16.54 | 5.36 | 23 |
| 18.17 | 4.88 | 64 |
| 18.79 | 4.72 | 100 |
| 19.27 | 4.61 | 44 |
| 20.64 | 4.30 | 33 |
| 20.93 | 4.25 | 33 |
| 21.51 | 4.13 | 26 |
| 22.18 | 4.01 | 24 |
| 22.65 | 3.93 | 27 |
| 23.83 | 3.73 | 19 |
| 24.29 | 3.66 | 15 |
| 24.57 | 3.62 | 15 |
| 25.53 | 3.49 | 10 |
| 26.72 | 3.34 | 9 |
| 27.56 | 3.24 | 8 |
| 28.32 | 3.15 | 7 |

### Acetate 4:

In an embodiment, provided herein is Acetate 4, which is a crystalline form of the acetate salt of a compound of Formula I, characterized by an X-ray powder diffraction pattern having peaks expressed in degrees-2-theta at angles (±0.2°) of 8.36, 17.74, 20.29, and 21.35. In particular aspects, Acetate 4 is characterized by an X-ray powder diffraction pattern having peaks expressed in degrees-2-theta at angles (±0.2°) of 7.10, 7.89, 8.36, 10.83, 11.45, 12.22, 13.60, 14.57, 15.51, 15.97, 17.00, 17.74, 18.23, 19.16, 19.84, 20.29, 20.81, 21.35, 22.05, 22.71, 23.10, 23.71, 24.26, 25.34, 26.16, and 26.84. In still more particular aspects, Acetate 4 is characterized by an X-ray powder diffraction pattern having peaks expressed in degrees-2-theta at angles (±0.2°) of 7.10, 7.89, 8.36, 9.09, 9.61, 10.30, 10.83, 11.45, 12.22, 12.89, 13.60, 14.57, 15.51, 15.97, 17.00, 17.74, 18.23, 19.16, 19.84, 20.29, 20.81, 21.35, 22.05, 22.71, 23.10, 23.71, 24.26, 25.34, 26.16, 26.8, 27.78, 28.39, 29.39, and 30.30. In another embodiment, the crystalline form of the compound of Formula I is Acetate 4, wherein the crystalline form is characterized by an X-ray powder diffraction pattern having peaks shown in Table 3 (expressed in degrees-2-theta at angles ±0.2°).

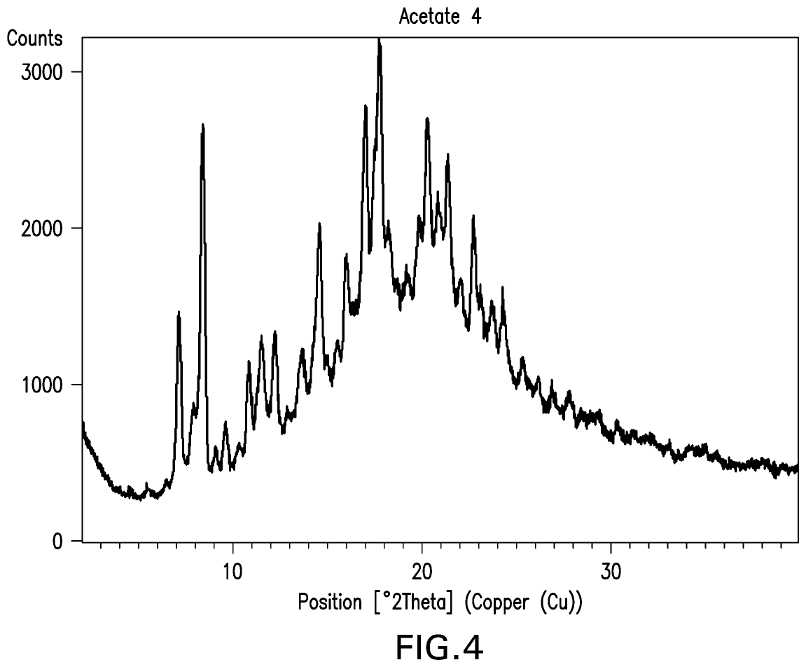

In aspects of this embodiment, about 10% to about 100% of the compound of Formula I in a pharmaceutical composition is in the form of Acetate 4, such as from about 25% to about 98%, from about 50% to about 96%, from about 75% to about 95%, from about 90% to about 94%, or about 92%. In aspects of this embodiment, Acetate 4 is characterized by an X-ray powder diffraction pattern substantially as shown in FIG. 4. In aspects of this embodiment, Acetate 4 is characterized by X-ray powder diffraction substantially as described by one or more of the characteristics recited in Table 3.

TABLE 3
X-Ray powder diffraction pattern of Acetate 4

| 2-θ ° | d-spacing, Å | Relative Intensity, % |
|---|---|---|
| 5.47 | 16.16 | 2 |
| 6.45 | 13.71 | 3 |
| 7.10 | 12.45 | 43 |
| 7.89 | 11.21 | 21 |
| 8.36 | 10.57 | 86 |
| 9.09 | 9.72 | 10 |
| 9.61 | 9.20 | 16 |
| 10.30 | 8.59 | 11 |
| 10.83 | 8.17 | 22 |
| 11.45 | 7.73 | 35 |
| 12.22 | 7.25 | 37 |
| 12.89 | 6.87 | 18 |
| 13.60 | 6.51 | 30 |
| 14.57 | 6.08 | 63 |
| 15.51 | 5.72 | 34 |
| 15.97 | 5.55 | 50 |
| 17.00 | 5.22 | 90 |
| 17.74 | 5.00 | 100 |
| 18.23 | 4.87 | 60 |
| 19.16 | 4.63 | 49 |
| 19.84 | 4.47 | 61 |
| 20.29 | 4.38 | 83 |
| 20.81 | 4.27 | 67 |
| 21.35 | 4.16 | 74 |
| 22.05 | 4.03 | 46 |
| 22.71 | 3.92 | 63 |
| 23.10 | 3.85 | 40 |
| 23.71 | 3.75 | 41 |
| 24.26 | 3.67 | 42 |
| 25.34 | 3.52 | 27 |
| 26.16 | 3.41 | 23 |
| 26.84 | 3.32 | 21 |
| 27.78 | 3.21 | 19 |
| 28.39 | 3.14 | 14 |
| 29.39 | 3.04 | 13 |
| 30.30 | 2.95 | 11 |

### Acetate 5:

In another embodiment, provided herein is Acetate 5, which is a crystalline form of the 5 acetate salt of a compound of Formula I, characterized by an X-ray powder diffraction pattern having peaks expressed in degrees-2-theta at angles (±0.2°) of 5.02, 6.66, 9.89, and 19.84. In particular aspects, Acetate 5 is characterized by an X-ray powder diffraction pattern having peaks expressed in degrees-2-theta at angles (±0.2°) of 5.02, 6.66, 9.89, 14.86, 16.32, 16.46, 16.91, 17.29, 17.54, 18.10, 18.59, 18.79, 19.12, 19.31, 19.65, 19.84, 20.38, 20.60, and 20.93. In still more particular aspects, Acetate 5 is characterized by an X-ray powder diffraction pattern having peaks expressed in degrees-2-theta at angles (±0.2°) of 5.02, 6.66, 9.89, 12.64, 13.33, 14.22, 14.47, 14.86, 15.08, 15.49, 15.77, 16.03, 16.32, 16.46, 16.91, 17.29, 17.54, 18.10, 18.59, 18.79, 19.12, 19.31, 19.65, 19.84, 20.38, 20.60, 20.93, 21.23, 21.65, 21.92, 22.33, 22.61, 22.95, and 23.38. In another embodiment, the crystalline form of the compound of Formula I is Acetate 5, wherein the crystalline form is characterized by an X-ray powder diffraction pattern having peaks shown in Table 4 (expressed in degrees-2-theta at angles ±0.2°).

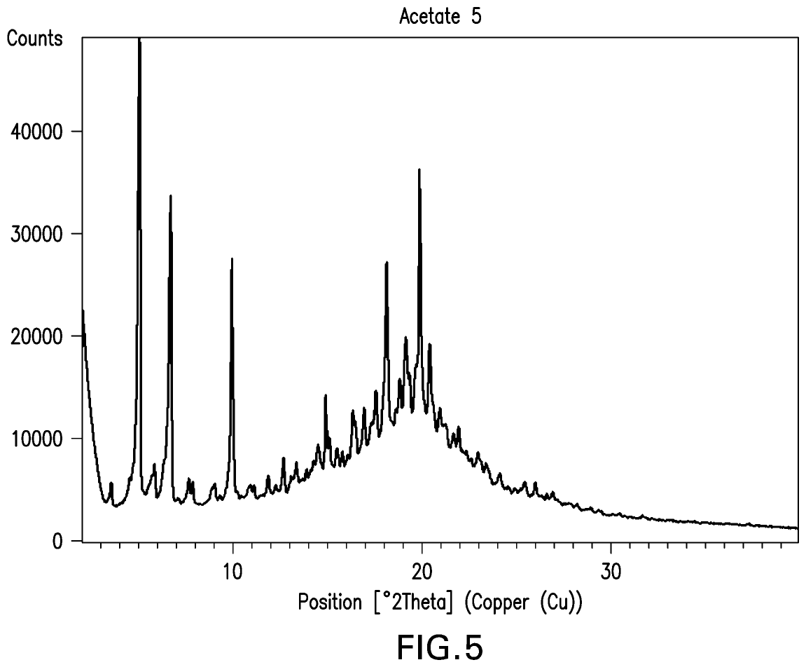

In aspects of this embodiment, about 10% to about 100% of the compound of Formula I in a pharmaceutical composition is in the form of Acetate 5, such as from about 25% to about 98%, from about 50% to about 96%, from about 75% to about 95%, from about 90% to about 94%, or about 92%. In aspects of this embodiment, Acetate 5 is characterized by an X-ray powder diffraction pattern substantially as shown in FIG. 5. In aspects of this embodiment, Acetate 5 is characterized by X-ray powder diffraction substantially as described by one or more of the characteristics recited in Table 4.

TABLE 4
X-Ray powder diffraction pattern of Acetate 5

| | | Relative |
|---|---|---|
| 2-θ ° | d-spacing, Å | Intensity, % |
| 3.53 | 25.02 | 3 |
| 4.49 | 19.70 | 6 |
| 5.02 | 17.60 | 100 |
| 5.64 | 15.67 | 6 |
| 5.80 | 15.24 | 9 |
| 6.31 | 14.01 | 9 |
| 6.66 | 13.26 | 62 |
| 7.08 | 12.49 | 2 |
| 7.63 | 11.58 | 6 |
| 7.83 | 11.30 | 5 |
| 8.85 | 9.99 | 4 |
| 8.99 | 9.83 | 5 |
| 9.28 | 9.53 | 2 |
| 9.89 | 8.94 | 54 |
| 10.20 | 8.67 | 4 |
| 10.41 | 8.50 | 3 |
| 10.88 | 8.13 | 5 |
| 11.09 | 7.98 | 5 |
| 11.83 | 7.48 | 7 |
| 12.24 | 7.23 | 5 |
| 12.64 | 7.01 | 11 |
| 13.07 | 6.77 | 7 |
| 13.33 | 6.64 | 10 |
| 13.85 | 6.40 | 8 |
| 14.05 | 6.30 | 9 |
| 14.22 | 6.23 | 11 |
| 14.47 | 6.12 | 14 |
| 14.86 | 5.96 | 25 |
| 15.08 | 5.88 | 16 |
| 15.49 | 5.72 | 14 |
| 15.77 | 5.62 | 13 |
| 16.03 | 5.53 | 12 |
| 16.32 | 5.43 | 22 |
| 16.46 | 5.39 | 19 |
| 16.91 | 5.24 | 23 |
| 17.29 | 5.13 | 20 |
| 17.54 | 5.06 | 27 |
| 18.10 | 4.90 | 55 |
| 18.59 | 4.77 | 23 |
| 18.79 | 4.72 | 29 |
| 19.12 | 4.64 | 38 |
| 19.31 | 4.60 | 30 |
| 19.65 | 4.52 | 32 |
| 19.84 | 4.47 | 75 |
| 20.38 | 4.36 | 37 |
| 20.60 | 4.31 | 23 |
| 20.93 | 4.24 | 23 |
| 21.23 | 4.19 | 19 |
| 21.65 | 4.10 | 18 |
| 21.92 | 4.06 | 19 |
| 22.33 | 3.98 | 14 |
| 22.61 | 3.93 | 12 |
| 22.95 | 3.88 | 14 |
| 23.38 | 3.81 | 11 |
| 24.10 | 3.69 | 9 |
| 24.58 | 3.62 | 6 |
| 24.91 | 3.57 | 6 |
| 25.43 | 3.50 | 7 |
| 25.44 | 3.51 | 8 |
| 25.98 | 3.43 | 8 |
| 26.57 | 3.35 | 5 |
| 26.87 | 3.32 | 6 |
| 27.88 | 3.20 | 3 |
| 28.21 | 3.16 | 3 |
| 28.93 | 3.08 | 3 |
| 29.32 | 3.04 | 2 |
| 30.45 | 2.93 | 1 |
| 30.75 | 2.91 | 1 |
| 31.67 | 2.82 | 1 |

### Acetate 6:

In another embodiment, provided herein is Acetate 6, which is a crystalline form of the 5 acetate salt of a compound of Formula I, characterized by an X-ray powder diffraction pattern having peaks expressed in degrees-2-theta at angles (±0.2°) of 7.79, 11.00, 16.24, and 18.89. In particular aspects, Acetate 6 is characterized by an X-ray powder diffraction pattern having peaks expressed in degrees-2-theta at angles (±0.2°) of 7.79, 11.00, 13.40, 14.70, 15.12, 15.44, 16.24, 17.05, 18.89, 20.34, and 20.96. In still more particular aspects, Acetate 6 is characterized by an X-ray powder diffraction pattern having peaks expressed in degrees-2-theta at angles (±0.2°) of 7.79, 9.47, 10.30, 11.00, 13.40, 14.01, 14.70, 15.12, 15.44, 16.24, 17.05, 18.89, 20.34, 20.96, and 21.95. In another embodiment, the crystalline form of the compound of Formula I is Acetate 6, wherein the crystalline form is characterized by an X-ray powder diffraction pattern having peaks shown in Table 5 (expressed in degrees-2-theta at angles ±0.2°).

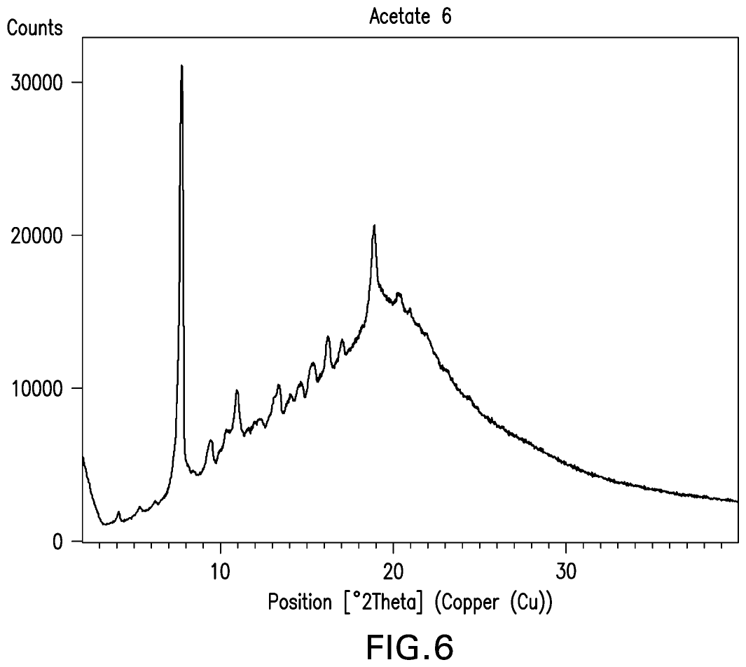

In aspects of this embodiment, about 10% to about 100% of the compound of Formula I in a pharmaceutical composition is in the form of Acetate 6, such as from about 25% to about 98%, from about 50% to about 96%, from about 75% to about 95%, from about 90% to about 94%, or about 92%. In aspects of this embodiment, Acetate 6 is characterized by an X-ray powder diffraction pattern substantially as shown in FIG. 6. In aspects of this embodiment, Acetate 6 is characterized by X-ray powder diffraction substantially as described by one or more of the characteristics recited in Table 5.

TABLE 5
X-Ray powder diffraction pattern of Acetate 6

| | | Relative |
|---|---|---|
| 2-θ ° | d-spacing, Å | Intensity, % |
| 4.12 | 21.47 | 2 |
| 5.31 | 16.63 | 2 |
| 6.18 | 14.30 | 2 |
| 7.79 | 11.34 | 100 |
| 9.47 | 9.33 | 11 |
| 10.30 | 8.59 | 12 |
| 11.00 | 8.04 | 20 |
| 12.31 | 7.19 | 11 |
| 13.40 | 6.61 | 18 |
| 14.01 | 6.32 | 13 |
| 14.70 | 6.02 | 15 |
| 15.12 | 5.86 | 18 |
| 15.44 | 5.74 | 19 |
| 16.24 | 5.46 | 25 |
| 17.05 | 5.20 | 22 |
| 18.89 | 4.70 | 49 |
| 20.34 | 4.37 | 28 |
| 20.96 | 4.24 | 22 |
| 21.95 | 4.05 | 13 |

### Caprate 2:

In another embodiment, provided herein is Caprate 2, which is a crystalline form of the caprate salt of a compound of Formula I, characterized by an X-ray powder diffraction pattern having peaks expressed in degrees-2-theta at angles (±0.2°) of 4.85, 7.65, 17.16, 18.20, and 19.50. In particular aspects, Caprate 2 is characterized by an X-ray powder diffraction pattern having peaks expressed in degrees-2-theta at angles (±0.2°) of 4.85, 6.27, 6.96, 7.65, 9.69, 17.16, 18.20, 19.50, 20.01, and 20.42. In another embodiment, the crystalline form of the compound of Formula I is Caprate 2, wherein the crystalline form is characterized by an X-ray powder diffraction pattern having peaks shown in Table 6 (expressed in degrees-2-theta at angles ±0.2°).

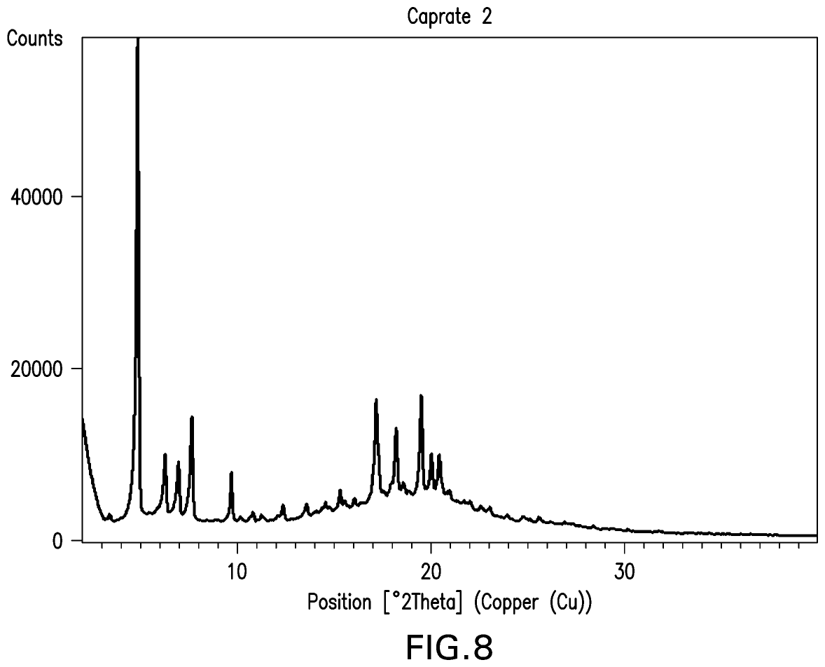

In aspects of this embodiment, about 10% to about 100% of the compound of Formula I in a pharmaceutical composition is in the form of Caprate 2, such as from about 25% to about 98%, from about 50% to about 96%, from about 75% to about 95%, from about 90% to about 94%, or about 92%. In aspects of this embodiment, Caprate 2 is characterized by an X-ray powder diffraction pattern substantially as shown in FIG. 8. In aspects of this embodiment, Caprate 2 is characterized by X-ray powder diffraction substantially as described by one or more of the characteristics recited in Table 6.

TABLE 6
X-Ray powder diffraction pattern of Caprate 2

| | | Relative |
|---|---|---|
| 2-θ ° | d-spacing, Å | Intensity, % |
| 3.43 | 25.78 | 1 |
| 4.85 | 18.23 | 100 |
| 6.27 | 14.10 | 14 |
| 6.96 | 12.70 | 12 |
| 7.65 | 11.56 | 22 |
| 8.93 | 9.90 | 0 |
| 9.69 | 9.13 | 10 |
| 10.15 | 8.71 | 1 |
| 10.80 | 8.19 | 2 |
| 11.24 | 7.87 | 2 |
| 12.07 | 7.33 | 2 |
| 12.37 | 7.15 | 4 |
| 13.57 | 6.52 | 4 |
| 14.55 | 6.09 | 5 |
| 14.78 | 5.99 | 3 |
| 15.31 | 5.79 | 7 |
| 15.54 | 5.70 | 5 |
| 16.06 | 5.52 | 6 |
| 17.16 | 5.17 | 26 |
| 17.95 | 4.94 | 9 |
| 18.20 | 4.87 | 21 |
| 18.55 | 4.78 | 9 |
| 18.87 | 4.70 | 7 |
| 19.50 | 4.55 | 27 |
| 20.01 | 4.44 | 15 |
| 20.42 | 4.35 | 15 |
| 20.95 | 4.24 | 8 |
| 21.67 | 4.10 | 5 |
| 22.06 | 4.03 | 5 |
| 22.56 | 3.94 | 5 |
| 23.07 | 3.86 | 4 |
| 23.91 | 3.72 | 3 |
| 24.76 | 3.60 | 3 |
| 25.09 | 3.55 | 2 |
| 25.54 | 3.49 | 2 |
| 26.16 | 3.41 | 2 |
| 26.91 | 3.31 | 2 |
| 28.37 | 3.15 | 1 |

### Caprate 3:

In another embodiment, provided herein is Caprate 3, which is a crystalline form of the caprate salt of a compound of Formula I, characterized by an X-ray powder diffraction pattern having peaks expressed in degrees-2-theta at angles (±0.2°) of 7.92, 17.33, and 19.60. In another embodiment, provided herein is Caprate 3, which is a crystalline form of the caprate salt of a compound of Formula I, characterized by an X-ray powder diffraction pattern having peaks expressed in degrees-2-theta at angles (±0.2°) of 7.92, 15.40, 17.33, and 19.60. In particular aspects, Caprate 3 is characterized by an X-ray powder diffraction pattern having peaks expressed in degrees-2-theta at angles (±0.2°) of 7.92, 15.40, 17.33, 18.86, 19.60, and 20.79. In still more particular aspects, Caprate 3 is characterized by an X-ray powder diffraction pattern having peaks expressed in degrees-2-theta at angles (±0.2°) of 7.92, 12.99, 15.40, 17.33, 18.59, 18.86, 19.07, 19.60, 20.79, and 21.27. In particular aspects, Caprate 3 is characterized by an X-ray powder diffraction pattern having peaks expressed in degrees-2-theta at angles (±0.2°) of 4.59, 7.92, 9.85, 12.99, 15.40, 16.66, 17.33, 18.59, 18.86, 19.07, 19.60, 20.79, 21.27, 21.73, and 22.24. In still more particular aspects, Caprate 3 is characterized by an X-ray powder diffraction pattern having peaks expressed in degrees-2-theta at angles (±0.2°) of 3.57, 4.59, 4.98, 7.92, 9.37, 9.85, 10.16, 10.38, 10.55, 11.35, 12.72, 12.99, 13.54, 13.75, 14.28, 14.66, 15.40, 16.66, 17.33, 17.97, 18.59, 18.86, 19.07, 19.60, 20.79, 21.27, 21.73, 22.24, 22.89, 23.64, 24.11, and 25.01. In another embodiment, the crystalline form of the compound of Formula I is Caprate 3, wherein the crystalline form is characterized by an X-ray powder diffraction pattern having peaks shown in Table 7 (expressed in degrees-2-theta at angles ±0.2°).

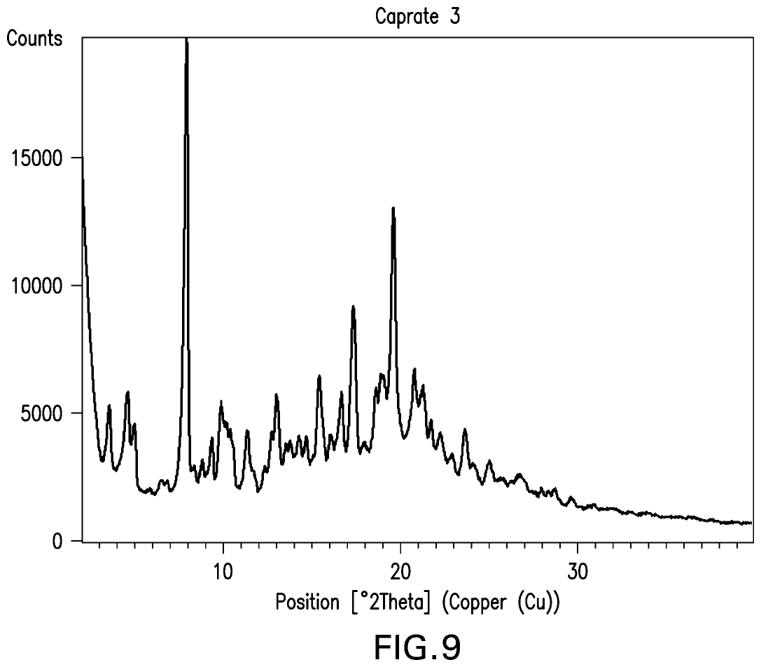

In aspects of this embodiment, about 10% to about 100% of the compound of Formula I in a pharmaceutical composition is in the form of Caprate 3, such as from about 25% to about 98%, from about 50% to about 96%, from about 75% to about 95%, from about 90% to about 94%, or about 92%. In aspects of this embodiment, Caprate 3 is characterized by an X-ray powder diffraction pattern substantially as shown in FIG. 9. In aspects of this embodiment, Caprate 3 is characterized by X-ray powder diffraction substantially as described by one or more of the characteristics recited in Table 7.

TABLE 7
X-Ray powder diffraction pattern of Caprate 3

| | | Relative |
|---|---|---|
| | d-spacing, | Intensity, |
| 2-θ ° | Å | % |
| 3.57 | 24.76 | 13 |
| 4.59 | 19.27 | 20 |
| 4.98 | 17.75 | 14 |
| 6.41 | 13.78 | 3 |
| 6.83 | 12.94 | 3 |
| 7.92 | 11.17 | 100 |
| 8.35 | 10.58 | 8 |
| 8.81 | 10.04 | 9 |
| 9.37 | 9.44 | 15 |
| 9.85 | 8.98 | 24 |
| 10.16 | 8.71 | 19 |
| 10.38 | 8.52 | 18 |
| 10.55 | 8.38 | 13 |
| 11.35 | 7.80 | 17 |
| 11.77 | 7.52 | 6 |
| 12.32 | 7.18 | 8 |
| 12.72 | 6.96 | 18 |
| 12.99 | 6.81 | 28 |
| 13.54 | 6.54 | 14 |
| 13.75 | 6.44 | 14 |
| 14.28 | 6.20 | 16 |
| 14.66 | 6.04 | 17 |
| 15.40 | 5.75 | 33 |
| 16.04 | 5.52 | 18 |
| 16.66 | 5.32 | 28 |
| 17.33 | 5.12 | 52 |
| 17.97 | 4.94 | 16 |
| 18.59 | 4.77 | 30 |
| 18.86 | 4.71 | 34 |
| 19.07 | 4.65 | 33 |
| 19.60 | 4.53 | 77 |
| 20.79 | 4.27 | 36 |
| 21.27 | 4.18 | 32 |
| 21.73 | 4.09 | 22 |
| 22.24 | 4.00 | 19 |
| 22.89 | 3.88 | 14 |
| 23.64 | 3.76 | 19 |
| 24.11 | 3.69 | 12 |
| 25.01 | 3.56 | 13 |
| 25.79 | 3.45 | 8 |
| 26.74 | 3.33 | 9 |
| 27.98 | 3.19 | 6 |
| 28.31 | 3.15 | 6 |
| 28.68 | 3.11 | 6 |
| 29.61 | 3.02 | 4 |
| 30.95 | 2.89 | 3 |
| 32.14 | 2.78 | 2 |
| 33.02 | 2.71 | 1 |
| 34.02 | 2.64 | 1 |

### Caprate 4:

In another embodiment, provided herein is Caprate 4, which is a crystalline form of the caprate salt of a compound of Formula I, characterized by an X-ray powder diffraction pattern having peaks expressed in degrees-2-theta at angles (±0.2°) of 4.18, 6.14, 17.51, and 17.68. In particular aspects, Caprate 4 is characterized by an X-ray powder diffraction pattern having peaks expressed in degrees-2-theta at angles (±0.2°) of 4.18, 4.86, 6.14, 8.41, 15.72, 16.90, 17.12, 17.51, 17.68, 18.17, 18.57, 19.21, 19.35, 19.96, 20.51, 20.87, 21.19, and 21.78. In another embodiment, the crystalline form of the compound of Formula I is Caprate 4, wherein the crystalline form is characterized by an X-ray powder diffraction pattern having peaks shown in Table 8 (expressed in degrees-2-theta at angles ±0.2°).

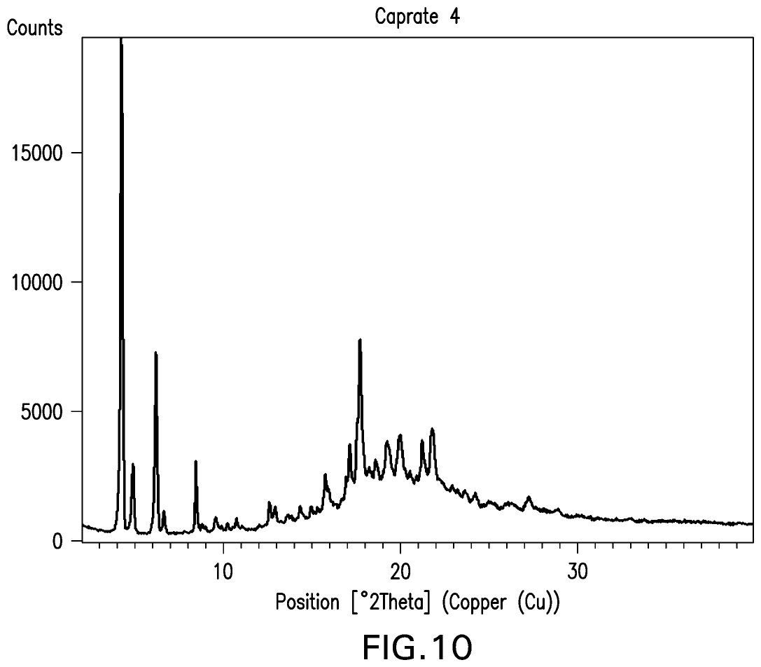

In aspects of this embodiment, about 10% to about 100% of the compound of Formula I in a pharmaceutical composition is in the form of Caprate 4, such as from about 25% to about 98%, from about 50% to about 96%, from about 75% to about 95%, from about 90% to about 94%, or about 92%. In aspects of this embodiment, Caprate 4 is characterized by an X-ray powder diffraction pattern substantially as shown in FIG. 10. In aspects of this embodiment, Caprate 4 is characterized by X-ray powder diffraction substantially as described by one or more of the characteristics recited in Table 8.

TABLE 8
X-Ray powder diffraction pattern of Caprate 4

| | d-spacing, | Relative |
|---|---|---|
| 2-θ ° | Å | Intensity, % |
| 4.18 | 21.13 | 100 |
| 4.86 | 18.20 | 14 |
| 6.14 | 14.39 | 38 |
| 6.59 | 13.41 | 5 |
| 8.41 | 10.51 | 15 |
| 8.77 | 10.08 | 2 |
| 8.98 | 9.85 | 1 |
| 9.52 | 9.29 | 3 |
| 9.86 | 8.97 | 1 |
| 10.20 | 8.68 | 2 |
| 10.70 | 8.27 | 3 |
| 11.05 | 8.01 | 1 |
| 11.97 | 7.39 | 1 |
| 12.56 | 7.05 | 6 |
| 12.88 | 6.87 | 5 |
| 13.61 | 6.51 | 3 |
| 14.30 | 6.20 | 5 |
| 14.92 | 5.94 | 5 |
| 15.28 | 5.80 | 4 |
| 15.72 | 5.64 | 12 |
| 15.92 | 5.57 | 8 |
| 16.90 | 5.25 | 11 |
| 17.12 | 5.18 | 18 |
| 17.51 | 5.06 | 21 |
| 17.68 | 5.02 | 39 |
| 18.17 | 4.88 | 13 |
| 18.57 | 4.78 | 14 |
| 19.21 | 4.62 | 18 |
| 19.35 | 4.59 | 16 |
| 19.96 | 4.45 | 19 |
| 20.51 | 4.33 | 12 |
| 20.87 | 4.26 | 11 |
| 21.19 | 4.19 | 18 |
| 21.78 | 4.08 | 19 |
| 22.88 | 3.89 | 9 |
| 23.56 | 3.78 | 7 |
| 24.23 | 3.67 | 6 |
| 24.93 | 3.57 | 5 |
| 26.00 | 3.43 | 4 |
| 27.21 | 3.28 | 6 |
| 28.90 | 3.09 | 3 |
| 29.93 | 2.99 | 2 |
| 30.67 | 2.92 | 1 |

### Caprate 5:

In another embodiment, provided herein is Caprate 5, which is a crystalline form of the caprate salt of a compound of Formula I, characterized by an X-ray powder diffraction pattern having peaks expressed in degrees-2-theta at angles (±0.2°) of 7.66, 16.18, 18.26, and 19.11. In particular aspects, Caprate 5 is characterized by having an X-ray powder diffraction pattern having peaks expressed in degrees-2-theta at angles (±0.2°) of 6.75, 7.66, 15.28, 16.18, 18.26, 19.11, and 20.63. In another embodiment, the crystalline form of the compound of Formula I is Caprate 5, wherein the crystalline form is characterized by an X-ray powder diffraction pattern having peaks shown in Table 9 (expressed in degrees-2-theta at angles ±0.2°).

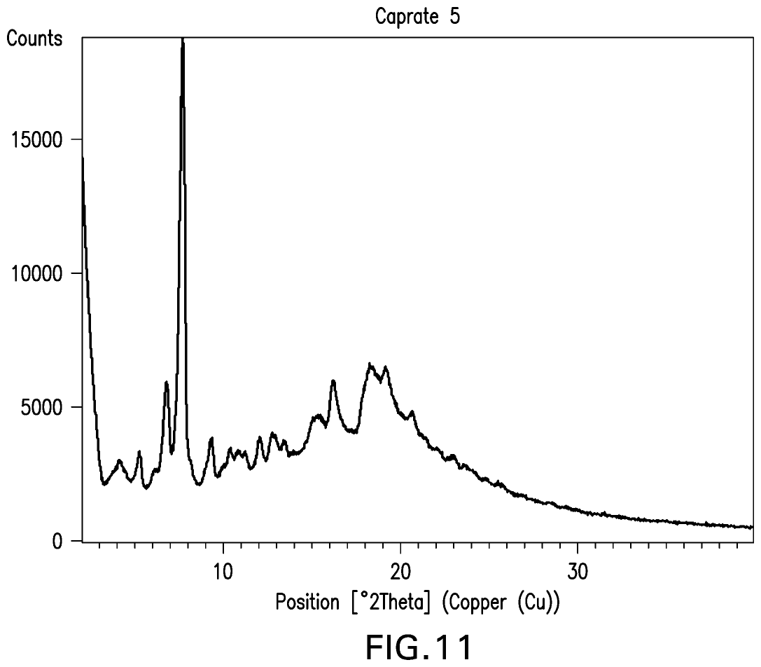

In aspects of this embodiment, about 10% to about 100% of the compound of Formula I in a pharmaceutical composition is in the form of Caprate 5, such as from about 25% to about 98%, from about 50% to about 96%, from about 75% to about 95%, from about 90% to about 94%, or about 92%. In aspects of this embodiment, Caprate 5 is characterized by an X-ray powder diffraction pattern substantially as shown in FIG. 11. In aspects of this embodiment, Caprate 5 is characterized by X-ray powder diffraction substantially as described by one or more of the characteristics recited in Table 9.

TABLE 9
X-Ray powder diffraction pattern of Caprate 5

| | | Relative |
|---|---|---|
| 2-θ ° | d-spacing, Å | Intensity, % |
| 4.12 | 21.43 | 5 |
| 5.21 | 16.96 | 8 |
| 6.07 | 14.57 | 3 |
| 6.75 | 13.10 | 28 |
| 7.66 | 11.54 | 100 |
| 9.29 | 9.52 | 13 |
| 10.32 | 8.57 | 10 |
| 10.80 | 8.19 | 10 |
| 11.24 | 7.87 | 10 |
| 12.06 | 7.34 | 14 |
| 12.80 | 6.92 | 15 |
| 13.43 | 6.60 | 14 |
| 15.28 | 5.80 | 21 |
| 16.18 | 5.48 | 30 |
| 18.26 | 4.86 | 36 |
| 19.11 | 4.65 | 36 |
| 20.63 | 4.31 | 24 |
| 21.96 | 4.05 | 14 |
| 22.99 | 3.87 | 13 |
| 23.61 | 3.77 | 10 |
| 25.67 | 3.47 | 6 |
| 28.39 | 3.14 | 2 |

### Caprate 6:

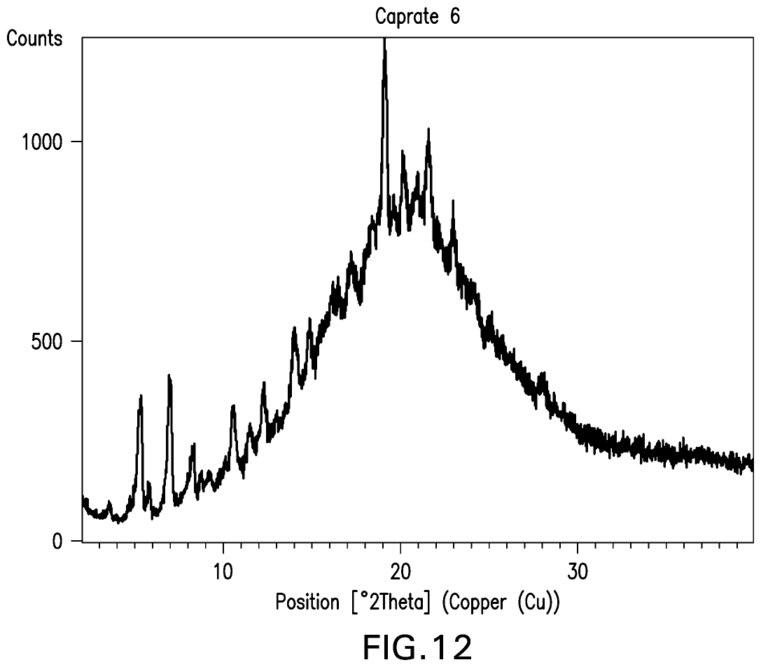

In another embodiment, provided herein is Caprate 6, which is a crystalline form of the caprate salt of a compound of Formula I, characterized by an X-ray powder diffraction pattern having peaks expressed in degrees-2-theta at angles (±0.2°) of 5.33, 6.97, 19.04, and 21.58. In particular aspects, Caprate 6 is characterized by having an X-ray powder diffraction pattern having peaks expressed in degrees-2-theta at angles (±0.2°) of 5.17, 5.33, 6.97, 10.47, 12.24, 13.97, 14.85, 16.23, 17.21, 18.40, 19.04, 20.08, 20.86, 21.58, 22.97, and 24.1. In another embodiment, the crystalline form of the compound of Formula I is Caprate 6, wherein the crystalline form is characterized by an X-ray powder diffraction pattern having peaks shown in Table 10 (expressed in degrees-2-theta at angles)+0.2°. In aspects of this embodiment, about 10% to about 100% of the compound of Formula I in a pharmaceutical composition is in the form of Caprate 6, such as from about 25% to about 98%, from about 50% to about 96%, from about 75% to about 95%, from about 90% to about 94%, or about 92%. In aspects of this embodiment, Caprate 6 is characterized by an X-ray powder diffraction pattern substantially as shown in FIG. 12. In aspects of this embodiment, Caprate 6 is characterized by X-ray powder diffraction substantially as described by one or more of the characteristics recited in Table 10.

TABLE 10
X-Ray powder diffraction pattern of Caprate 6

| | | Relative |
|---|---|---|
| 2-θ ° | d-spacing, Å | Intensity, % |
| 3.52 | 25.13 | 4 |
| 5.17 | 17.09 | 28 |
| 5.33 | 16.58 | 32 |
| 5.81 | 15.22 | 6 |
| 6.97 | 12.68 | 34 |
| 8.25 | 10.72 | 13 |
| 8.75 | 10.10 | 5 |
| 9.24 | 9.57 | 3 |
| 10.47 | 8.45 | 20 |
| 11.45 | 7.73 | 12 |
| 12.24 | 7.23 | 22 |
| 13.97 | 6.34 | 33 |
| 14.85 | 5.97 | 37 |
| 16.23 | 5.46 | 39 |
| 17.21 | 5.15 | 48 |
| 18.40 | 4.82 | 55 |
| 19.04 | 4.66 | 100 |
| 20.08 | 4.42 | 69 |
| 20.86 | 4.26 | 61 |
| 21.58 | 4.12 | 72 |
| 22.97 | 3.87 | 49 |
| 24.19 | 3.68 | 23 |
| 27.98 | 3.19 | 4 |

### Caprate 7:

In another embodiment, provided herein is Caprate 7, which is a crystalline form of the caprate salt of a compound of Formula I, characterized by an X-ray powder diffraction pattern 7.73, 17.14, 18.75, and 19.48. In another embodiment, the crystalline form of the compound of Formula I is Caprate 7, wherein the crystalline form is characterized by an X-ray powder diffraction pattern having peaks shown in Table 11 (expressed in degrees-2-theta at angles ±0.2°).

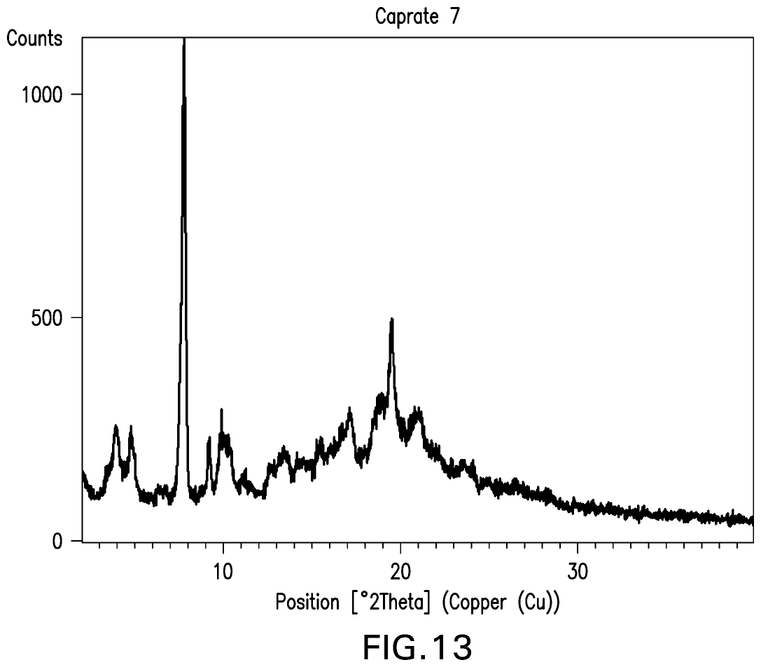

In aspects of this embodiment, about 10% to about 100% of the compound of Formula I in a pharmaceutical composition is in the form of Caprate 7, such as from about 25% to about 98%, from about 50% to about 96%, from about 75% to about 95%, from about 90% to about 94%, or about 92%. In aspects of this embodiment, Caprate 7 is characterized by an X-ray powder diffraction pattern substantially as shown in FIG. 13. In aspects of this embodiment, Caprate 7 is characterized by X-ray powder diffraction substantially as described by one or more of the characteristics recited in Table 11.

TABLE 11
X-Ray powder diffraction pattern of Caprate 7

| | | Relative |
|---|---|---|
| 2-θ ° | d-spacing, Å | Intensity, % |
| 3.90 | 22.66 | 15 |
| 4.74 | 18.65 | 14 |
| 7.73 | 11.44 | 100 |
| 9.15 | 9.67 | 12 |
| 9.83 | 9.00 | 13 |
| 10.36 | 8.54 | 10 |
| 11.16 | 7.93 | 8 |
| 12.59 | 7.03 | 5 |
| 13.49 | 6.56 | 8 |
| 14.23 | 6.22 | 6 |
| 15.41 | 5.75 | 9 |
| 17.14 | 5.17 | 17 |
| 18.75 | 4.73 | 17 |
| 19.48 | 4.56 | 37 |
| 20.94 | 4.24 | 16 |
| 23.53 | 3.78 | 4 |

### Caprate 8:

In another embodiment, provided herein is Caprate 8, which is a crystalline form of the caprate salt of a compound of Formula I, characterized by an X-ray powder diffraction pattern 7.45, 17.97, 19.32, and 22.08. In particular aspects, Caprate 8 is characterized by having an X-ray powder diffraction pattern having peaks expressed in degrees-2-theta at angles (±0.2°) of 6.35, 7.45, 14.95, 16.13, 17.46, 17.97, 19.32, 20.62, and 22.08. In another embodiment, the crystalline form of the compound of Formula I is Caprate 8, wherein the crystalline form is characterized by an X-ray powder diffraction pattern having peaks shown in Table 12 (expressed in degrees-2-theta at angles ±0.2°).

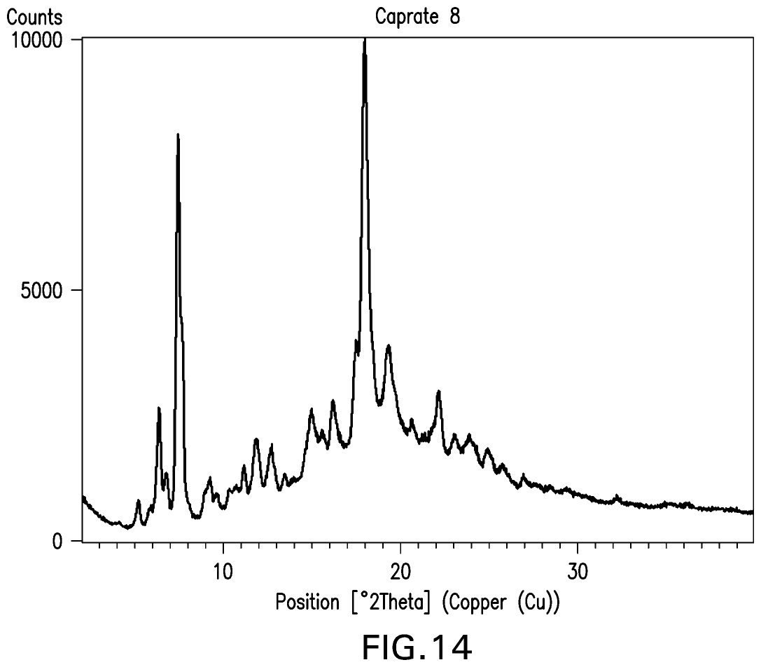

In aspects of this embodiment, about 10% to about 100% of the compound of Formula I in a pharmaceutical composition is in the form of Caprate 8, such as from about 25% to about 98%, from about 50% to about 96%, from about 75% to about 95%, from about 90% to about 94%, or about 92%. In aspects of this embodiment, Caprate 8 is characterized by an X-ray powder diffraction pattern substantially as shown in FIG. 14. In aspects of this embodiment, Caprate 8 is characterized by X-ray powder diffraction substantially as described by one or more of the characteristics recited in Table 12.

TABLE 12
X-Ray powder diffraction pattern of Caprate 8

| | | Relative |
|---|---|---|
| | d-spacing, | Intensity, |
| 2-θ ° | Å | % |
| 4.11 | 21.49 | 1 |
| 5.20 | 17.00 | 6 |
| 5.89 | 15.01 | 4 |
| 6.35 | 13.92 | 25 |
| 6.76 | 13.07 | 11 |
| 7.45 | 11.87 | 82 |
| 7.68 | 11.52 | 42 |
| 8.92 | 9.91 | 7 |
| 9.21 | 9.60 | 9 |
| 9.66 | 9.16 | 6 |
| 10.31 | 8.58 | 7 |
| 10.67 | 8.29 | 8 |
| 11.17 | 7.92 | 12 |
| 11.85 | 7.47 | 17 |
| 12.69 | 6.98 | 15 |
| 13.47 | 6.57 | 10 |
| 14.95 | 5.93 | 23 |
| 15.56 | 5.69 | 19 |
| 16.13 | 5.50 | 25 |
| 17.46 | 5.08 | 37 |
| 17.97 | 4.93 | 100 |
| 19.32 | 4.59 | 35 |
| 20.62 | 4.31 | 21 |
| 22.08 | 4.03 | 26 |
| 23.03 | 3.86 | 17 |
| 23.85 | 3.73 | 17 |
| 24.90 | 3.58 | 14 |
| 25.70 | 3.47 | 11 |
| 26.92 | 3.31 | 8 |
| 28.41 | 3.14 | 6 |
| 29.34 | 3.04 | 5 |
| 32.18 | 2.78 | 3 |

### Caprate 9:

In another embodiment, provided herein is Caprate 9, which is a crystalline form of the caprate salt of Formula I, characterized by an X-ray powder diffraction pattern 6.73, 11.95, 18.23, and 19.77. In particular aspects, Caprate 9 is characterized by having an X-ray powder diffraction pattern having peaks expressed in degrees-2-theta at angles (±0.2°) of 5.01, 6.73, 11.33, 11.95, 12.67, 13.05, 13.43, 13.85, 14.05, 14.34, 15.19, 15.61, 16.54, 16.81, 17.03, 17.53, 17.64, 18.23, 18.56, 19.37, 19.57, 19.77, 20.21, 20.39, 20.53, 21.05, 21.83, 22.14, 22.77, 23.12, 23.69, 23.95, 24.62, 25.07, 25.47, and 26.08. In another embodiment, the crystalline form of the compound of Formula I is Caprate 9, wherein the crystalline form is characterized by an X-ray powder diffraction pattern having peaks shown in Table 13 (expressed in degrees-2-theta at angles ±0.2°).

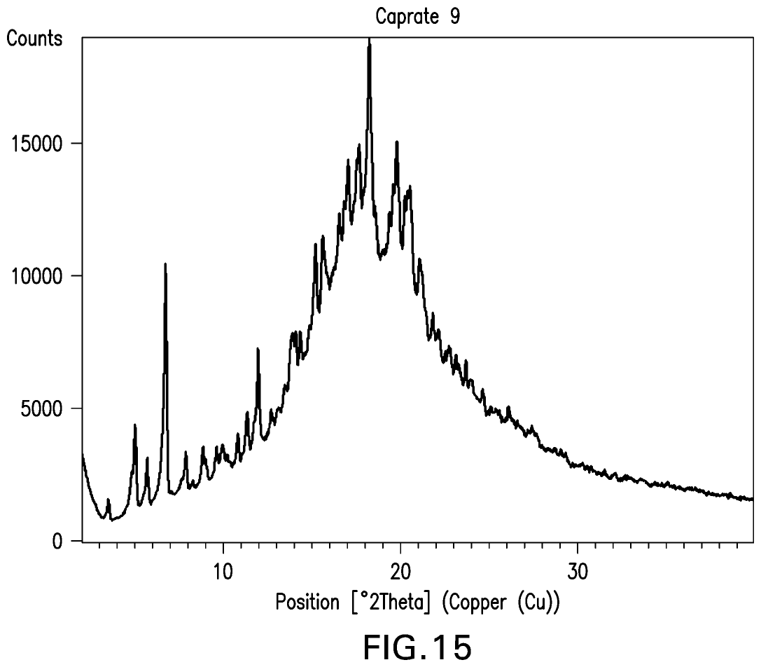

5 In aspects of this embodiment, about 10% to about 100% of the compound of Formula I in a pharmaceutical composition is in the form of Caprate 9, such as from about 25% to about 98%, from about 50% to about 96%, from about 75% to about 95%, from about 90% to about 94%, or about 92%. In aspects of this embodiment, Caprate 9 is characterized by an X-ray powder diffraction pattern substantially as shown in FIG. 15. In aspects of this embodiment, Caprate 9 is characterized by X-ray powder diffraction substantially as described by one or more of the characteristics recited in Table 13.

TABLE 13
X-Ray powder diffraction pattern of Caprate 9

| | | Relative |
|---|---|---|
| | d-spacing, | Intensity, |
| 2-θ ° | Å | % |
| 3.50 | 25.24 | 3 |
| 4.81 | 18.36 | 10 |
| 5.01 | 17.63 | 20 |
| 5.69 | 15.53 | 13 |
| 6.73 | 13.13 | 53 |
| 7.86 | 11.25 | 14 |
| 8.25 | 10.71 | 7 |
| 8.84 | 10.01 | 15 |
| 9.01 | 9.82 | 12 |
| 9.59 | 9.22 | 14 |
| 9.95 | 8.89 | 15 |
| 10.21 | 8.67 | 12 |
| 10.81 | 8.18 | 17 |
| 11.33 | 7.81 | 22 |
| 11.95 | 7.41 | 35 |
| 12.67 | 6.99 | 22 |
| 13.05 | 6.79 | 22 |
| 13.43 | 6.59 | 27 |
| 13.85 | 6.39 | 36 |
| 14.05 | 6.30 | 38 |
| 14.34 | 6.18 | 38 |
| 15.19 | 5.83 | 56 |
| 15.61 | 5.68 | 58 |
| 16.54 | 5.36 | 63 |
| 16.81 | 5.27 | 64 |
| 17.03 | 5.21 | 73 |
| 17.53 | 5.06 | 74 |
| 17.64 | 5.03 | 77 |
| 18.23 | 4.87 | 100 |
| 18.56 | 4.78 | 64 |
| 19.37 | 4.58 | 63 |
| 19.57 | 4.54 | 69 |
| 19.77 | 4.49 | 78 |
| 20.21 | 4.39 | 66 |
| 20.39 | 4.36 | 67 |
| 20.53 | 4.33 | 68 |
| 21.05 | 4.22 | 53 |
| 21.83 | 4.07 | 41 |
| 22.14 | 4.02 | 38 |
| 22.77 | 3.90 | 34 |
| 23.12 | 3.85 | 32 |
| 23.69 | 3.76 | 31 |
| 23.95 | 3.72 | 27 |
| 24.62 | 3.62 | 24 |
| 25.07 | 3.55 | 21 |
| 25.47 | 3.50 | 20 |
| 26.08 | 3.42 | 21 |
| 26.76 | 3.33 | 17 |
| 27.42 | 3.25 | 17 |
| 28.70 | 3.11 | 12 |
| 29.01 | 3.08 | 11 |
| 29.33 | 3.04 | 11 |
| 30.15 | 2.96 | 8 |
| 31.50 | 2.84 | 7 |
| 32.08 | 2.79 | 6 |
| 32.66 | 2.74 | 5 |
| 33.62 | 2.67 | 5 |
| 35.10 | 2.56 | 4 |

### Caprate 10:

In another embodiment, provided herein is Caprate 10, which is a crystalline form of the caprate salt of a compound of Formula I, characterized by an X-ray powder diffraction pattern 3.50, 7.90, 16.21, and 18.23. In another embodiment, the crystalline form of the compound of Formula I is Caprate 10, wherein the crystalline form is characterized by an X-ray powder diffraction pattern having peaks shown in Table 14 (expressed in degrees-2-theta at angles ±0.2°).

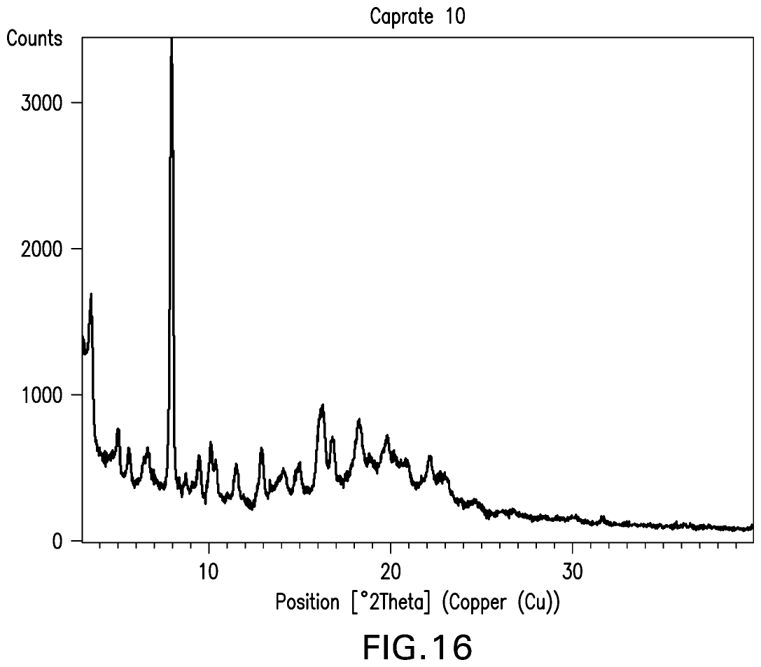

In aspects of this embodiment, about 10% to about 100% of the compound of Formula I in a pharmaceutical composition is in the form of Caprate 10, such as from about 25% to about 98%, from about 50% to about 96%, from about 75% to about 95%, from about 90% to about 94%, or about 92%. In aspects of this embodiment, Caprate 10 is characterized by an X-ray powder diffraction pattern substantially as shown in FIG. 16. In aspects of this embodiment, Caprate 10 is characterized by X-ray powder diffraction substantially as described by one or more of the characteristics recited in Table 14.

TABLE 14
X-Ray powder diffraction pattern of Caprate 10

| | | Relative |
|---|---|---|
| | d-spacing, | Intensity, |
| 2-θ ° | Å | % |
| 3.50 | 25.27 | 48 |
| 5.01 | 17.63 | 19 |
| 5.56 | 15.89 | 15 |
| 6.40 | 13.81 | 13 |
| 6.60 | 13.39 | 15 |
| 7.90 | 11.19 | 100 |
| 8.69 | 10.18 | 9 |
| 9.42 | 9.38 | 13 |
| 10.06 | 8.79 | 15 |
| 10.34 | 8.55 | 11 |
| 11.47 | 7.72 | 12 |
| 12.88 | 6.88 | 15 |
| 14.05 | 6.30 | 10 |
| 14.92 | 5.94 | 11 |
| 15.98 | 5.55 | 18 |
| 16.21 | 5.47 | 24 |
| 16.76 | 5.29 | 17 |
| 18.23 | 4.87 | 21 |
| 19.76 | 4.49 | 17 |
| 20.90 | 4.25 | 12 |
| 22.12 | 4.02 | 13 |
| 22.66 | 3.92 | 15 |
| 23.00 | 3.87 | 9 |
| 24.60 | 3.62 | 4 |
| 26.03 | 3.42 | 2 |
| 26.69 | 3.34 | 2 |

### Caprate 11:

In another embodiment, provided herein is Caprate 11, which is a crystalline form of the 5 caprate salt of a compound of Formula I, characterized by an X-ray powder diffraction pattern 3.93, 4.90, and 7.68. In another embodiment, the crystalline form of the compound of Formula I is Caprate 11, wherein the crystalline form is characterized by an X-ray powder diffraction pattern having peaks shown in Table 15 (expressed in degrees-2-theta at angles ±0.2°).

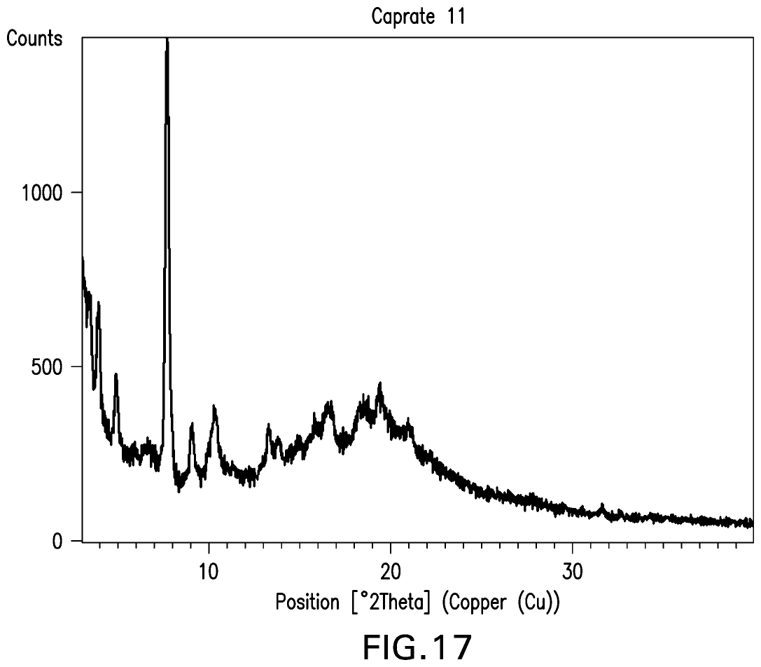

In aspects of this embodiment, about 10% to about 100% of the compound of Formula I in a pharmaceutical composition is in the form of Caprate 11, such as from about 25% to about 98%, from about 50% to about 96%, from about 75% to about 95%, from about 90% to about 94%, or about 92%. In aspects of this embodiment, Caprate 11 is characterized by an X-ray powder diffraction pattern substantially as shown in FIG. 17. In aspects of this embodiment, Caprate 11 is characterized by X-ray powder diffraction substantially as described by one or more of the characteristics recited in Table 15.

TABLE 15
X-Ray powder diffraction pattern of Caprate 11

| | | Relative |
|---|---|---|
| | d-spacing, | Intensity, |
| 2-θ, ° | Å | % |
| 3.93 | 22.50 | 41 |
| 4.90 | 18.05 | 25 |
| 6.54 | 13.52 | 7 |
| 7.68 | 11.51 | 100 |
| 9.04 | 9.78 | 12 |
| 10.30 | 8.59 | 16 |
| 13.25 | 6.68 | 12 |
| 13.82 | 6.41 | 9 |
| 15.78 | 5.62 | 12 |
| 16.56 | 5.35 | 14 |
| 18.28 | 4.85 | 15 |
| 19.31 | 4.60 | 18 |
| 20.95 | 4.24 | 10 |

### Caprate 12:

In another embodiment, provided herein is Caprate 12, which is a crystalline form of the caprate salt of a compound of Formula I, characterized by an X-ray powder diffraction pattern 6.80, 15.37, 18.22, and 20.63. In particular aspects, Caprate 12 is characterized by having an X-ray powder diffraction pattern having peaks expressed in degrees-2-theta at angles (±0.2°) of 5.01, 5.58, 6.80, 10.75, 13.44, 13.85, 14.43, 15.37, 16.00, 16.34, 16.68, 17.67, 18.22, 18.50, 19.09, 19.67, 20.27, 20.63, 21.33, 22.30, 23.22, 23.88, 25.39, and 26.02. In another embodiment, the crystalline form of the compound of Formula I is Caprate 12, wherein the crystalline form is characterized by an X-ray powder diffraction pattern having peaks shown in Table 16 (expressed in degrees-2-theta at angles ±0.2°).

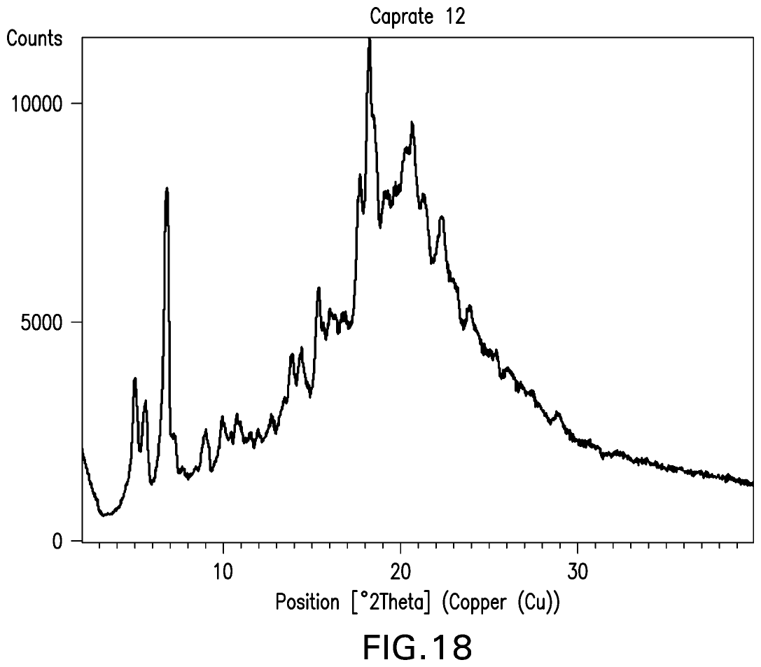

In aspects of this embodiment, about 10% to about 100% of the compound of Formula I in a pharmaceutical composition is in the form of Caprate 12, such as from about 25% to about 98%, from about 50% to about 96%, from about 75% to about 95%, from about 90% to about 94%, or about 92%. In aspects of this embodiment, Caprate 12 is characterized by an X-ray powder diffraction pattern substantially as shown in FIG. 18. In aspects of this embodiment, Caprate 12 is characterized by X-ray powder diffraction substantially as described by one or more of the characteristics recited in Table 16.

TABLE 16
X-Ray powder diffraction pattern of Caprate 12

| | | Relative |
|---|---|---|
| 2-θ ° | d-spacing, Å | Intensity, % |
| 5.01 | 17.64 | 29 |
| 5.58 | 15.82 | 24 |
| 6.80 | 13.01 | 63 |
| 7.21 | 12.25 | 16 |
| 7.69 | 11.50 | 9 |
| 8.98 | 9.85 | 17 |
| 9.17 | 9.65 | 12 |
| 9.92 | 8.92 | 19 |
| 10.75 | 8.23 | 20 |
| 11.57 | 7.65 | 15 |
| 11.93 | 7.42 | 16 |
| 12.72 | 6.96 | 19 |
| 13.44 | 6.59 | 22 |
| 13.85 | 6.39 | 31 |
| 14.43 | 6.14 | 32 |
| 15.37 | 5.77 | 46 |
| 16.00 | 5.54 | 40 |
| 16.34 | 5.42 | 39 |
| 16.68 | 5.31 | 39 |
| 17.67 | 5.02 | 62 |
| 18.22 | 4.87 | 100 |
| 18.50 | 4.80 | 75 |
| 19.09 | 4.65 | 65 |
| 19.67 | 4.51 | 66 |
| 20.27 | 4.38 | 74 |
| 20.63 | 4.31 | 80 |
| 21.33 | 4.17 | 64 |
| 22.30 | 3.99 | 59 |
| 23.22 | 3.83 | 42 |
| 23.88 | 3.73 | 38 |
| 25.39 | 3.51 | 27 |
| 26.02 | 3.42 | 24 |
| 27.41 | 3.25 | 18 |
| 28.83 | 3.10 | 13 |
| 31.16 | 2.87 | 4 |

### Caprate 13:

In another embodiment, provided herein is Caprate 13, which is a crystalline form of the 5 caprate salt of a compound of Formula I, characterized by an X-ray powder diffraction pattern 5.02, 6.29, 7.12, and 20.25. In particular aspects, Caprate 13 is characterized by having an X-ray powder diffraction pattern having peaks expressed in degrees-2-theta at angles (±0.2°) of 4.23, 5.02, 6.29, 7.12, 15.16, 16.47, 16.97, 17.33, 18.12, 18.88, 19.09, 20.25, 21.53, 22.08, and 23.06. In another embodiment, the crystalline form the compound of Formula I is Caprate 13, wherein the crystalline form is characterized by an X-ray powder diffraction pattern having peaks shown 10 in Table 17 (expressed in degrees-2-theta at angles ±0.2°).

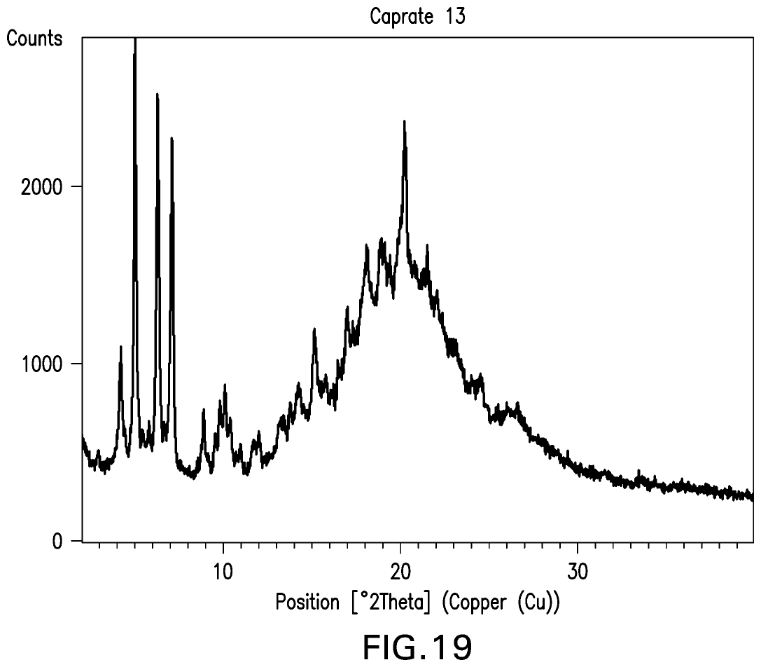

In aspects of this embodiment, about 10% to about 100% of the compound of Formula I in a pharmaceutical composition is in the form of Caprate 13, such as from about 25% to about 98%, from about 50% to about 96%, from about 75% to about 95%, from about 90% to about 94%, or about 92%. In aspects of this embodiment, Caprate 13 is characterized by an X-ray powder diffraction pattern substantially as shown in FIG. 19. In aspects of this embodiment, Caprate 13 is characterized by X-ray powder diffraction substantially as described by one or more of the characteristics recited in Table 17.

TABLE 17
X-Ray powder diffraction pattern of Caprate 13

| | | Relative |
|---|---|---|
| 2-θ ° | d-spacing, Å | Intensity, % |
| 2.97 | 29.79 | 5 |
| 4.23 | 20.89 | 29 |
| 5.02 | 17.59 | 100 |
| 5.45 | 16.20 | 10 |
| 5.81 | 15.21 | 11 |
| 6.29 | 14.05 | 83 |
| 7.12 | 12.42 | 77 |
| 8.90 | 9.94 | 14 |
| 9.56 | 9.25 | 8 |
| 9.83 | 9.00 | 15 |
| 10.10 | 8.76 | 19 |
| 10.41 | 8.50 | 12 |
| 10.98 | 8.06 | 5 |
| 11.68 | 7.58 | 5 |
| 12.04 | 7.35 | 7 |
| 13.17 | 6.72 | 9 |
| 13.79 | 6.42 | 14 |
| 14.27 | 6.21 | 17 |
| 15.16 | 5.85 | 30 |
| 15.79 | 5.61 | 19 |
| 16.47 | 5.38 | 23 |
| 16.97 | 5.22 | 33 |
| 17.33 | 5.12 | 30 |
| 18.12 | 4.90 | 48 |
| 18.88 | 4.70 | 49 |
| 19.09 | 4.65 | 47 |
| 20.25 | 4.39 | 77 |
| 21.53 | 4.13 | 46 |
| 22.08 | 4.03 | 33 |
| 23.06 | 3.86 | 23 |
| 24.54 | 3.63 | 15 |
| 25.48 | 3.50 | 8 |
| 26.59 | 3.35 | 8 |
| 33.56 | 2.67 | 1 |

### Caprate 14:

In another embodiment, provided herein is Caprate 14, which is a crystalline form of the caprate salt of a compound of Formula I, characterized by an X-ray powder diffraction pattern 6.74, 18.16, 19.51, and 20.68. In particular aspects, Caprate 14 is characterized by having an X-ray powder diffraction pattern having peaks expressed in degrees-2-theta at angles (±0.2°) of 5.01, 5.54, 6.74, 7.06, 15.29, 16.08, 16.64, 17.67, 18.16, 18.54, 19.13, 19.51, 20.68, 21.40, 22.26, and 23.22. In another embodiment, the crystalline form of the compound of Formula I is Caprate 14, wherein the crystalline form is characterized by an X-ray powder diffraction pattern having peaks shown in Table 18 (expressed in degrees-2-theta at angles ±0.2°).

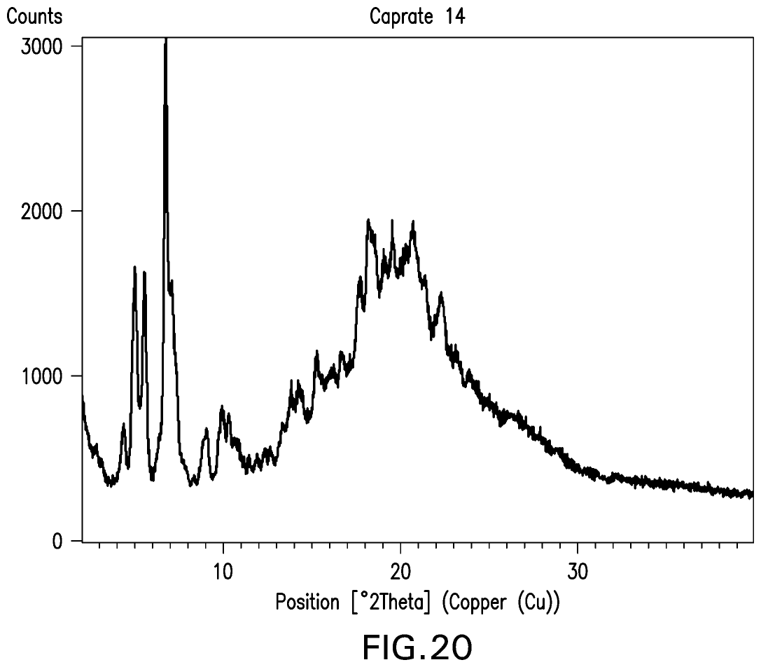

In aspects of this embodiment, about 10% to about 100% of the compound of Formula I in a pharmaceutical composition is in the form of Caprate 14, such as from about 25% to about 98%, from about 50% to about 96%, from about 75% to about 95%, from about 90% to about 94%, or about 92%. In aspects of this embodiment, Caprate 14 is characterized by an X-ray powder diffraction pattern substantially as shown in FIG. 20. In aspects of this embodiment, Caprate 14 is characterized by X-ray powder diffraction substantially as described by one or more of the characteristics recited in Table 18.

TABLE 18
X-Ray powder diffraction pattern of Caprate 14

| | | Relative |
|---|---|---|
| | d-spacing, | Intensity, |
| 2-θ, ° | Å | % |
| 2.81 | 31.40 | 8 |
| 4.36 | 20.28 | 11 |
| 5.01 | 17.65 | 47 |
| 5.54 | 15.96 | 47 |
| 6.74 | 13.12 | 100 |
| 7.06 | 12.53 | 43 |
| 7.35 | 12.03 | 19 |
| 9.05 | 9.77 | 9 |
| 9.92 | 8.91 | 14 |
| 10.31 | 8.58 | 13 |
| 10.80 | 8.19 | 6 |
| 11.43 | 7.74 | 3 |
| 11.85 | 7.47 | 2 |
| 12.31 | 7.19 | 4 |
| 12.64 | 7.00 | 4 |
| 13.31 | 6.65 | 9 |
| 13.84 | 6.40 | 18 |
| 14.24 | 6.22 | 18 |
| 15.29 | 5.80 | 25 |
| 16.08 | 5.51 | 20 |
| 16.64 | 5.33 | 24 |
| 17.67 | 5.02 | 38 |
| 18.16 | 4.89 | 50 |
| 18.54 | 4.79 | 49 |
| 19.13 | 4.64 | 44 |
| 19.51 | 4.55 | 51 |
| 20.68 | 4.30 | 51 |
| 21.40 | 4.15 | 38 |
| 22.26 | 3.99 | 35 |
| 23.22 | 3.83 | 20 |
| 23.83 | 3.73 | 16 |

### D-Lactate 1:

In another embodiment, provided herein is D-Lactate 1, which is a crystalline form of the lactate salt of a compound of Formula I, characterized by an X-ray powder diffraction pattern having peaks expressed in degrees-2-theta at angles (±0.2°) of 18.24, 19.56, 20.07, and 20.45. In particular aspects, D-Lactate 1 is characterized by having an X-ray powder diffraction pattern having peaks expressed in degrees-2-theta at angles (±0.2°) of 17.26, 18.24, 19.56, 20.07, 20.45, 20.89, 21.72, and 22.10. In still more particular aspects, D-Lactate 1 is characterized by having an X-ray powder diffraction pattern having peaks expressed in degrees-2-theta at angles (±0.2°) of 13.74, 14.54, 16.09, 17.26, 18.24, 19.56, 20.07, 20.45, 20.89, 21.72, and 22.10. In another embodiment, the crystalline form of the compound of Formula I is D-Lactate 1, wherein the crystalline form is characterized by an X-ray powder diffraction pattern having peaks shown in Table 19 (expressed in degrees-2-theta at angles ±0.2°).

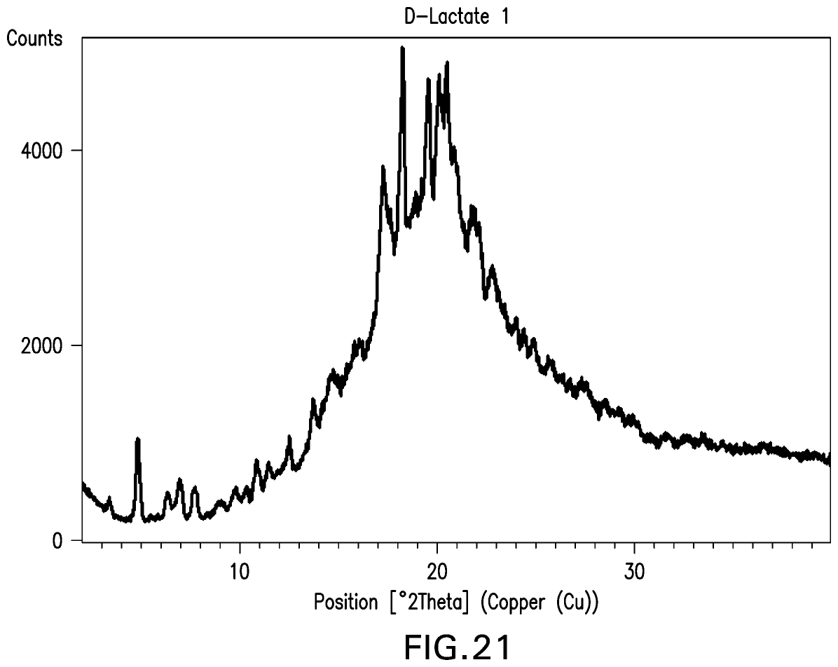

In aspects of this embodiment, about 10% to about 100% of the compound of Formula I in a pharmaceutical composition is in the form of D-Lactate 1, such as from about 25% to about 98%, from about 50% to about 96%, from about 75% to about 95%, from about 90% to about 94%, or about 92%. In aspects of this embodiment, D-Lactate 1 is characterized by an X-ray powder diffraction pattern substantially as shown in FIG. 21. In aspects of this embodiment, D-Lactate 1 is characterized by X-ray powder diffraction substantially as described by one or more of the characteristics recited in Table 19.

TABLE 19
X-Ray powder diffraction pattern of D-Lactate 1

| | | Relative |
|---|---|---|
| | d-spacing, | Intensity, |
| 2-θ ° | Å | % |
| 3.41 | 25.88 | 3 |
| 4.85 | 18.24 | 16 |
| 6.34 | 13.94 | 6 |
| 6.95 | 12.72 | 8 |
| 7.74 | 11.42 | 5 |
| 9.08 | 9.74 | 3 |
| 9.79 | 9.04 | 5 |
| 10.36 | 8.54 | 5 |
| 10.86 | 8.14 | 11 |
| 11.45 | 7.73 | 10 |
| 12.53 | 7.07 | 15 |
| 13.74 | 6.45 | 23 |
| 14.54 | 6.09 | 28 |
| 16.09 | 5.51 | 35 |
| 17.26 | 5.14 | 73 |
| 18.24 | 4.86 | 100 |
| 19.56 | 4.54 | 92 |
| 20.07 | 4.42 | 92 |
| 20.45 | 4.34 | 96 |
| 20.89 | 4.25 | 76 |
| 21.72 | 4.09 | 61 |
| 22.10 | 4.02 | 59 |
| 22.80 | 3.90 | 48 |
| 24.03 | 3.70 | 36 |
| 24.39 | 3.65 | 33 |
| 24.89 | 3.58 | 32 |
| 25.62 | 3.48 | 26 |
| 27.42 | 3.25 | 20 |
| 28.46 | 3.14 | 16 |
| 29.23 | 3.06 | 14 |
| 30.06 | 2.97 | 11 |
| 31.58 | 2.83 | 7 |
| 32.67 | 2.74 | 6 |
| 33.42 | 2.68 | 6 |
| 34.47 | 2.60 | 4 |

### D-Lactate 2:

In another embodiment, provided herein is D-Lactate 2, which is a crystalline form of the 5 lactate salt of a compound of Formula I, characterized by an X-ray powder diffraction pattern having peaks expressed in degrees-2-theta at angles (±0.2°) of 7.38. In particular aspects, D-Lactate 2 is characterized by having an X-ray powder diffraction pattern having peaks expressed in degrees-2-theta at angles (±0.2°) of 7.38 and 19.63. In another embodiment, the crystalline form of the compound of Formula I is D-Lactate 2, wherein the crystalline form is characterized by an X-ray powder diffraction pattern having peaks shown in Table 20 (expressed in degrees-2-theta at angles ±0.2°).

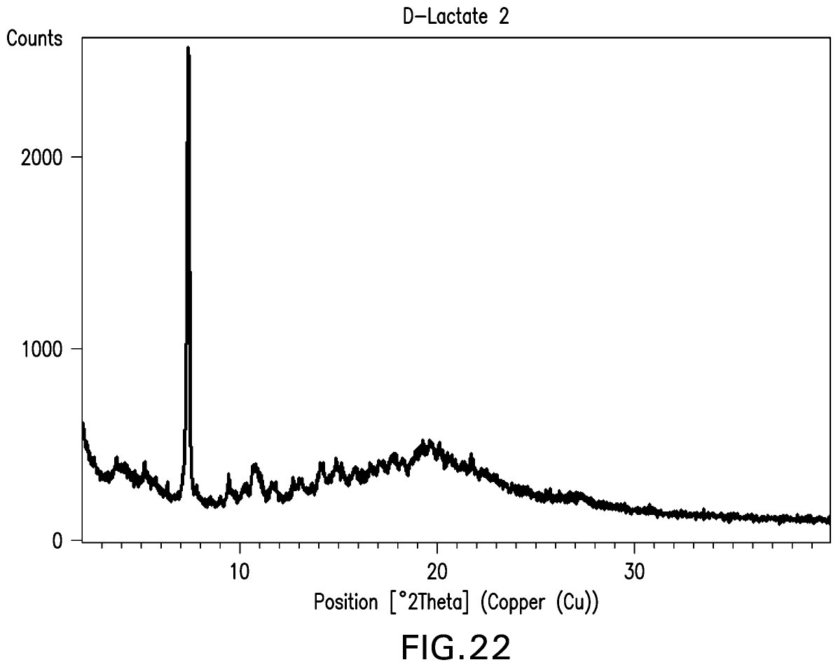

In aspects of this embodiment, about 10% to about 100% of the compound of Formula I in a pharmaceutical composition is in the form of D-Lactate 2, such as from about 25% to about 98%, from about 50% to about 96%, from about 75% to about 95%, from about 90% to about 94%, or about 92%. In aspects of this embodiment, D-Lactate 2 is characterized by an X-ray powder diffraction pattern substantially as shown in FIG. 22. In aspects of this embodiment, D-Lactate 2 is characterized by X-ray powder diffraction substantially as described by one or more of the characteristics recited in Table 20.

TABLE 20
X-Ray powder diffraction pattern of D-Lactate 2

| | | Relative |
|---|---|---|
| | d-spacing, | Intensity, |
| 2-θ ° | Å | % |
| 3.71 | 23.84 | 4 |
| 5.15 | 17.16 | 6 |
| 7.38 | 11.97 | 100 |
| 9.42 | 9.39 | 5 |
| 10.22 | 8.65 | 3 |
| 10.69 | 8.27 | 8 |
| 11.59 | 7.63 | 3 |
| 12.67 | 6.99 | 4 |
| 13.07 | 6.77 | 4 |
| 14.14 | 6.26 | 7 |
| 14.87 | 5.96 | 8 |
| 15.83 | 5.60 | 6 |
| 16.59 | 5.34 | 7 |
| 17.06 | 5.20 | 7 |
| 17.71 | 5.01 | 9 |
| 18.21 | 4.87 | 8 |
| 18.98 | 4.67 | 9 |
| 19.63 | 4.52 | 11 |
| 20.08 | 4.42 | 9 |
| 20.54 | 4.32 | 8 |
| 21.69 | 4.10 | 6 |

### Succinate 1:

In another embodiment, provided herein is Succinate 1, which is a crystalline form of the succinate salt of a compound of Formula I, characterized by an X-ray powder diffraction pattern having peaks expressed in degrees-2-theta at angles (±0.2°) of 5.98, 7.05, 17.29, and 20.22. In particular aspects, Succinate 1 is characterized by having an X-ray powder diffraction pattern having peaks expressed in degrees-2-theta at angles (±0.2°) of 5.98, 7.05, 17.29, 18.82, 20.22, and 21.39. In still more particular aspects, Succinate 1 is characterized by having an X-ray powder diffraction pattern having peaks expressed in degrees-2-theta at angles (±0.2°) of 4.84, 5.49, 5.98, 7.05, 14.38, 16.79, 17.29, 18.82, 20.22, and 21.39. In another embodiment, the crystalline form of the compound of Formula I is Succinate 1, wherein the crystalline form is characterized by an X-ray powder diffraction pattern having peaks shown in Table 21 (expressed in degrees-2-theta at angles ±0.2°).

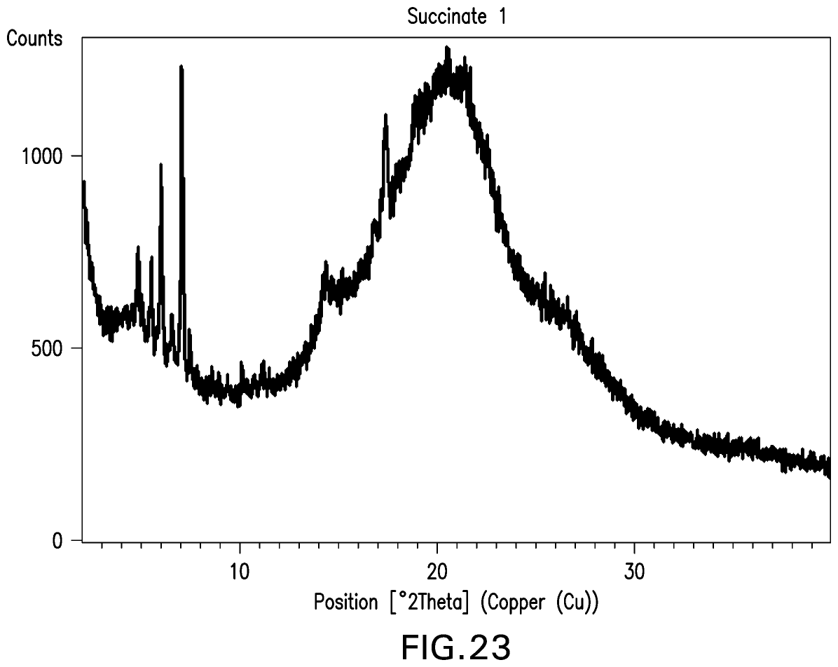

In aspects of this embodiment, about 10% to about 100% of the compound of Formula I in a pharmaceutical composition is in the form of Succinate 1, such as from about 25% to about 98%, from about 50% to about 96%, from about 75% to about 95%, from about 90% to about 94%, or about 92%. In aspects of this embodiment, Succinate 1 is characterized by an X-ray powder diffraction pattern substantially as shown in FIG. 23. In aspects of this embodiment, Succinate 1 is characterized by X-ray powder diffraction substantially as described by one or more of the characteristics recited in Table 21.

TABLE 21
X-Ray powder diffraction pattern of Succinate 1

| | | Relative |
|---|---|---|
| | d-spacing, | Intensity, |
| 2-θ ° | Å | % |
| 4.84 | 18.25 | 28 |
| 5.49 | 16.09 | 28 |
| 5.98 | 14.77 | 63 |
| 6.53 | 13.53 | 15 |
| 7.05 | 12.55 | 100 |
| 7.46 | 11.85 | 12 |
| 10.10 | 8.75 | 6 |
| 11.10 | 7.97 | 6 |
| 14.38 | 6.16 | 21 |
| 16.79 | 5.28 | 24 |
| 17.29 | 5.13 | 50 |
| 18.82 | 4.72 | 45 |
| 20.22 | 4.39 | 51 |
| 21.39 | 4.15 | 47 |
| 26.75 | 3.33 | 4 |

### Succinate 2:

In another embodiment, provided herein is Succinate 2, which is a crystalline form of the succinate salt of a compound of Formula I, characterized by an X-ray powder diffraction pattern having peaks expressed in degrees-2-theta at angles (±0.2°) of 5.15, 6.12, 7.22, and 7.90. In another embodiment, the crystalline form of the compound of Formula I is Succinate 2, wherein the crystalline form is characterized by an X-ray powder diffraction pattern having peaks shown in Table 22 (expressed in degrees-2-theta at angles ±0.2°).

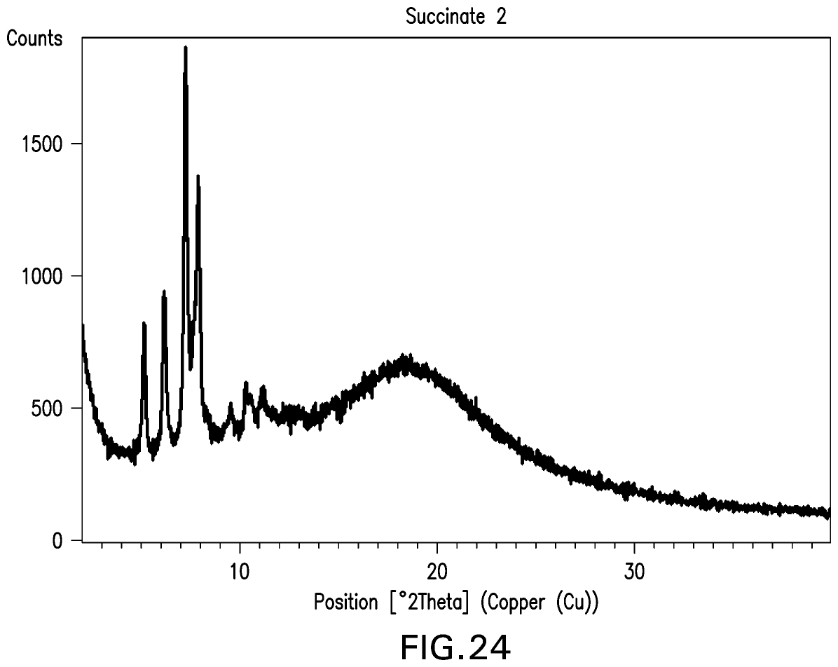

In aspects of this embodiment, about 10% to about 100% of the compound of Formula I in a pharmaceutical composition is in the form of Succinate 2, such as from about 25% to about 98%, from about 50% to about 96%, from about 75% to about 95%, from about 90% to about 94%, or about 92%. In aspects of this embodiment, Succinate 2 is characterized by an X-ray powder diffraction pattern substantially as shown in FIG. 24. In aspects of this embodiment, Succinate 2 is characterized by X-ray powder diffraction substantially as described by one or more of the characteristics recited in Table 22.

TABLE 22
X-Ray powder diffraction pattern of Succinate 2

| | | Relative |
|---|---|---|
| | d-spacing, | Intensity, |
| 2-θ ° | Å | % |
| 5.15 | 17.16 | 33 |
| 6.12 | 14.45 | 38 |
| 7.22 | 12.25 | 100 |
| 7.90 | 11.19 | 66 |
| 9.55 | 9.27 | 6 |
| 10.28 | 8.60 | 11 |
| 11.16 | 7.93 | 8 |

### L-Tartrate 1:

In another embodiment, provided herein is L-Tartrate 1, which is a crystalline form of the tartrate salt of a compound of Formula I, characterized by an X-ray powder diffraction pattern having peaks expressed in degrees-2-theta at angles (±0.2°) of 4.71, 6.52, 7.48, and 17.37. In another embodiment, the crystalline form of the compound of Formula I is L-Tartrate 1, wherein the crystalline form is characterized by an X-ray powder diffraction pattern having peaks shown in Table 23 (expressed in degrees-2-theta at angles ±0.2°).

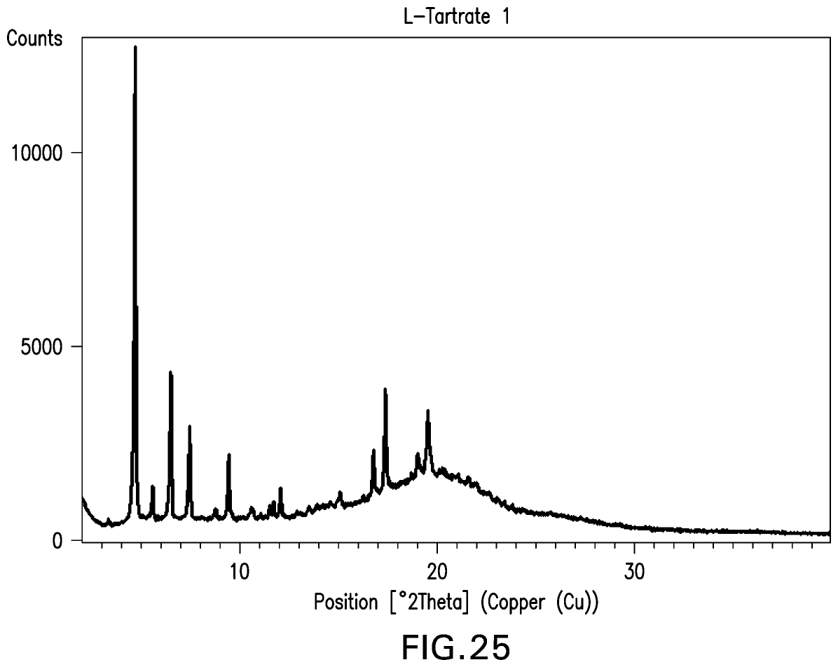

In aspects of this embodiment, about 10% to about 100% of the compound of Formula I in a pharmaceutical composition is in the form of L-Tartrate 1, such as from about 25% to about 98%, from about 50% to about 96%, from about 75% to about 95%, from about 90% to about 94%, or about 92%. In aspects of this embodiment, L-Tartrate 1 is characterized by an X-ray powder diffraction pattern substantially as shown in FIG. 25. In aspects of this embodiment, L-Tartrate 1 is characterized by X-ray powder diffraction substantially as described by one or more of the characteristics recited in Table 23.

TABLE 23
X-Ray powder diffraction pattern of L-Tartrate 1

| | d-spacing, | Relative |
|---|---|---|
| 2-θ ° | Å | Intensity, % |
| 4.71 | 18.75 | 100 |
| 5.61 | 15.75 | 8 |
| 6.52 | 13.56 | 31 |
| 7.48 | 11.82 | 20 |
| 8.81 | 10.04 | 3 |
| 9.45 | 9.36 | 15 |
| 10.62 | 8.33 | 3 |
| 11.07 | 8.00 | 2 |
| 11.53 | 7.67 | 4 |
| 11.73 | 7.54 | 4 |
| 12.07 | 7.33 | 7 |
| 13.51 | 6.56 | 3 |
| 13.93 | 6.36 | 4 |
| 14.58 | 6.08 | 4 |
| 15.09 | 5.87 | 6 |
| 16.27 | 5.45 | 5 |
| 16.77 | 5.29 | 15 |
| 17.37 | 5.10 | 28 |
| 18.70 | 4.75 | 10 |
| 19.00 | 4.67 | 13 |
| 19.54 | 4.54 | 23 |
| 20.34 | 4.37 | 10 |
| 21.08 | 4.22 | 9 |
| 21.59 | 4.12 | 9 |
| 21.98 | 4.04 | 8 |
| 22.63 | 3.93 | 5 |
| 23.03 | 3.86 | 5 |
| 23.39 | 3.80 | 4 |
| 23.81 | 3.74 | 3 |
| 24.38 | 3.65 | 2 |

### L-Tartrate 2:

In another embodiment, provided herein is L-Tartrate 2, which is a crystalline form of the tartrate salt of a compound of Formula I, characterized by an X-ray powder diffraction pattern having peaks expressed in degrees-2-theta at angles (±0.2°) of 7.35, 14.19, 15.86, and 18.70. In particular aspects, L-Tartrate 2 is characterized by having an X-ray powder diffraction pattern having peaks expressed in degrees-2-theta at angles (±0.2°) of 7.35, 10.90, 14.19, 15.86, 16.63, 17.58, 18.20, 18.70, 19.79, 20.54, and 20.94. In another embodiment, the crystalline form of the compound of Formula I is L-Tartrate 2, wherein the crystalline form is characterized by an X-ray 10 powder diffraction pattern having peaks shown in Table 24 (expressed in degrees-2-theta at angles ±0.2°).

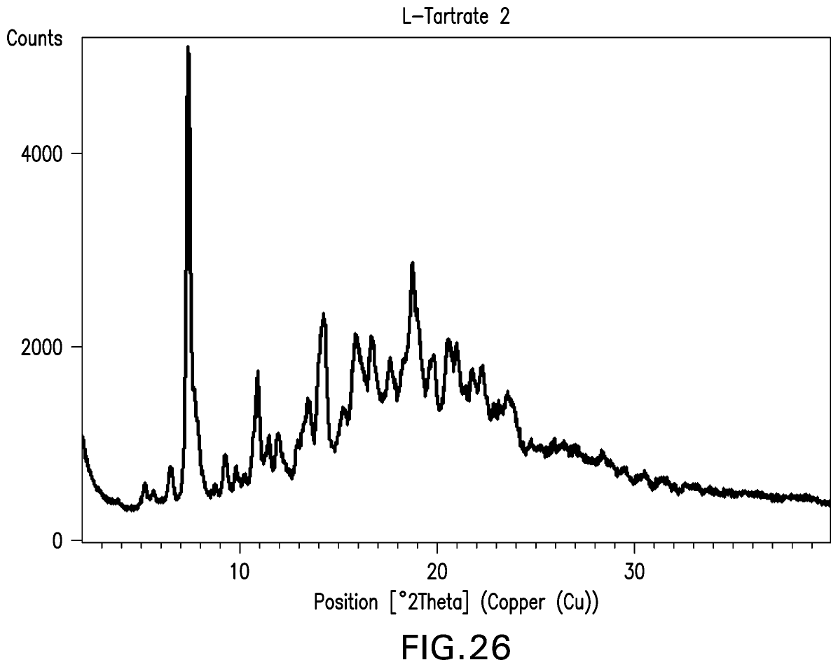

In aspects of this embodiment, about 10% to about 100% of the compound of Formula I in a pharmaceutical composition is in the form of L-Tartrate 2, such as from about 25% to about 98%, from about 50% to about 96%, from about 75% to about 95%, from about 90% to about 94%, or about 92%. In aspects of this embodiment, L-Tartrate 2 is characterized by an X-ray powder diffraction pattern substantially as shown in FIG. 26. In aspects of this embodiment, L-Tartrate 2 is characterized by X-ray powder diffraction substantially as described by one or more of the characteristics recited in Table 24.

TABLE 24
X-Ray powder diffraction pattern of L-Tartrate 2

| | d-spacing, | Relative |
|---|---|---|
| 2-θ ° | Å | Intensity, % |
| 5.17 | 17.08 | 5 |
| 5.61 | 15.75 | 3 |
| 6.46 | 13.67 | 9 |
| 7.35 | 12.03 | 100 |
| 7.88 | 11.22 | 17 |
| 8.72 | 10.14 | 4 |
| 9.27 | 9.54 | 11 |
| 9.78 | 9.05 | 8 |
| 10.23 | 8.65 | 6 |
| 10.90 | 8.12 | 30 |
| 11.45 | 7.73 | 15 |
| 11.88 | 7.45 | 15 |
| 12.81 | 6.91 | 12 |
| 13.42 | 6.60 | 23 |
| 14.19 | 6.24 | 40 |
| 15.16 | 5.84 | 20 |
| 15.86 | 5.59 | 37 |
| 16.63 | 5.33 | 36 |
| 17.58 | 5.04 | 31 |
| 18.20 | 4.87 | 30 |
| 18.70 | 4.74 | 53 |
| 19.79 | 4.49 | 32 |
| 20.54 | 4.32 | 33 |
| 20.94 | 4.24 | 34 |
| 21.72 | 4.09 | 28 |
| 22.24 | 4.00 | 29 |
| 22.85 | 3.89 | 21 |
| 23.52 | 3.78 | 24 |
| 23.84 | 3.73 | 20 |
| 24.70 | 3.61 | 12 |
| 25.82 | 3.45 | 12 |
| 26.36 | 3.38 | 12 |
| 27.02 | 3.30 | 10 |
| 28.37 | 3.15 | 9 |
| 29.40 | 3.04 | 6 |
| 30.46 | 2.93 | 5 |
| 31.49 | 2.84 | 3 |
| 32.50 | 2.75 | 2 |

### Sulfate 1:

In another embodiment, provided herein is Sulfate 1, which is a crystalline form of the 5 sulfate salt of a compound of Formula I, characterized by an X-ray powder diffraction pattern having peaks expressed in degrees-2-theta at angles (±0.2°) of 6.87, 19.48, 20.38, and 20.94. In particular aspects, Sulfate 1 is characterized by having an X-ray powder diffraction pattern having peaks expressed in degrees-2-theta at angles (±0.2°) of 2.40, 6.87, 9.13, 17.83, 18.50, 19.48, 20.38, 20.94, and 21.94. In another embodiment, the crystalline form of the compound of Formula I is Sulfate 1, wherein the crystalline form is characterized by an X-ray powder diffraction pattern having peaks shown in Table 25 (expressed in degrees-2-theta at angles ±0.2°).

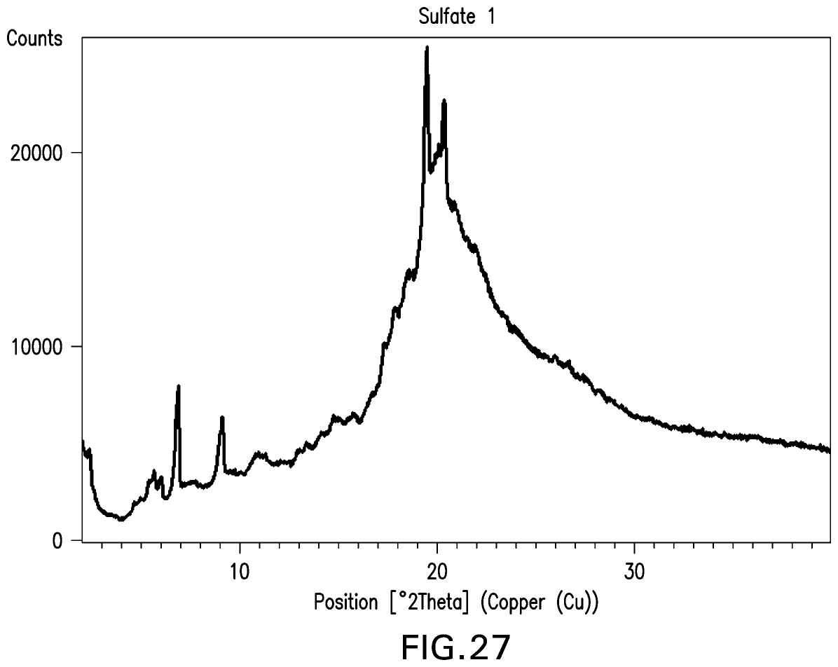

In aspects of this embodiment, about 10% to about 100% of the compound of Formula I in a pharmaceutical composition is in the form of Sulfate 1, such as from about 25% to about 98%, from about 50% to about 96%, from about 75% to about 95%, from about 90% to about 94%, or about 92%. In aspects of this embodiment, Sulfate 1 is characterized by an X-ray powder diffraction pattern substantially as shown in FIG. 27. In aspects of this embodiment, Sulfate 1 is characterized by X-ray powder diffraction substantially as described by one or more of the characteristics recited in Table 25.

TABLE 25
X-Ray powder diffraction pattern of Sulfate 1

| | | Relative |
|---|---|---|
| | d-spacing, | Intensity, |
| 2-θ ° | Å | % |
| 2.40 | 36.87 | 21 |
| 4.65 | 18.99 | 4 |
| 5.37 | 16.45 | 9 |
| 5.66 | 15.61 | 11 |
| 6.03 | 14.65 | 8 |
| 6.87 | 12.87 | 34 |
| 7.68 | 11.51 | 4 |
| 9.13 | 9.69 | 21 |
| 10.82 | 8.17 | 6 |
| 11.28 | 7.85 | 5 |
| 13.02 | 6.80 | 2 |
| 13.34 | 6.64 | 2 |
| 14.11 | 6.28 | 2 |
| 14.73 | 6.01 | 4 |
| 17.30 | 5.13 | 18 |
| 17.83 | 4.98 | 26 |
| 18.50 | 4.80 | 34 |
| 19.48 | 4.56 | 100 |
| 20.38 | 4.36 | 77 |
| 20.94 | 4.24 | 43 |
| 21.94 | 4.05 | 27 |

### Sulfate 2:

In another embodiment, provided herein is Sulfate 2, which is a crystalline form of the sulfate salt of a compound of Formula I, characterized by an X-ray powder diffraction pattern having peaks expressed in degrees-2-theta at angles (±0.2°) of 5.21, 5.75, 6.30, and 7.74. In another embodiment, the crystalline form of the compound of Formula I is Sulfate 2, wherein the crystalline form is characterized by an X-ray powder diffraction pattern having peaks shown in Table 26 (expressed in degrees-2-theta at angles ±0.2°).

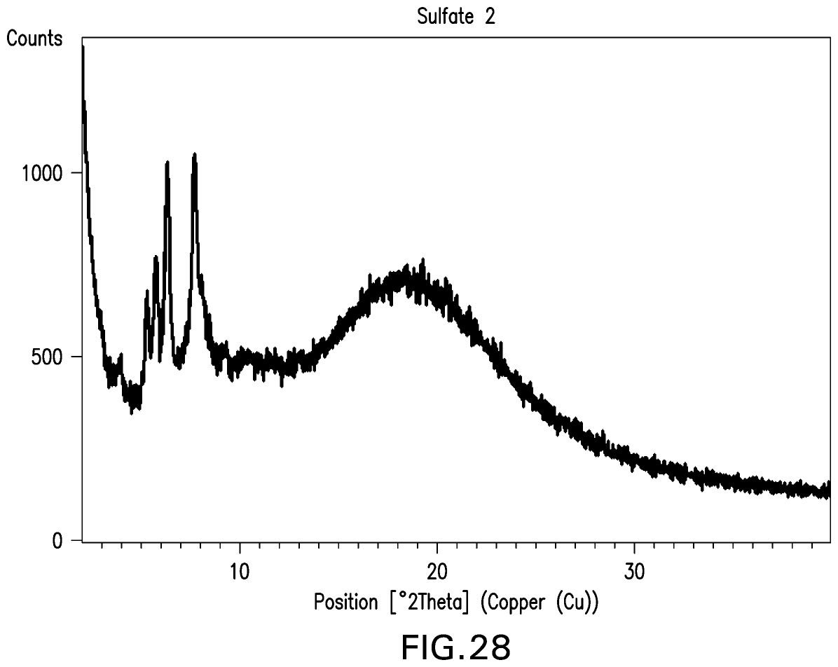

In aspects of this embodiment, about 10% to about 100% of the compound of Formula I in a pharmaceutical composition is in the form of Sulfate 2, such as from about 25% to about 98%, from about 50% to about 96%, from about 75% to about 95%, from about 90% to about 94%, or about 92%. In aspects of this embodiment, Sulfate 2 is characterized by an X-ray powder diffraction pattern substantially as shown in FIG. 28. In aspects of this embodiment, Sulfate 2 is characterized by X-ray powder diffraction substantially as described by one or more of the characteristics recited in Table 26.

TABLE 26
X-Ray powder diffraction pattern of Sulfate 2

| | | Relative |
|---|---|---|
| | d-spacing, | Intensity, |
| 2-θ ° | Å | % |
| 3.96 | 22.30 | 9 |
| 5.21 | 16.96 | 33 |
| 5.75 | 15.36 | 57 |
| 6.30 | 14.03 | 100 |
| 7.74 | 11.43 | 94 |
| 9.20 | 9.62 | 8 |

Additional aspects of such embodiments provide a particular drug substance that comprises a crystalline form of a compound of Formula I as described herein. By “drug substance” is meant the active pharmaceutical ingredient. The presence of a crystalline form in a drug substance can be detected by physical methods known to those of ordinary skill in the art, such as X-ray powder diffraction, carbon-13 cross-polarization magic-angle spinning (CPMAS) nuclear magnetic resonance (NMR) spectroscopy, and nitrogen-15 CPMAS NMR spectroscopy.

In additional aspects of this embodiment, Compound 2 is crystallized from a solvent system comprising a solvent chosen from ethers, esters, straight chained alkanes (C₃-C₁₀), alcohol, and water. In additional aspects of this embodiment, Compound 2 is crystallized from a solvent system comprising a solvent selected from the group consisting of ethers, esters, straight chained alkanes (C₃-C₁₀), alcohol, and water and mixtures thereof. In some embodiments, Compound 2 is crystallized from a solvent system comprising a solvent selected from the group consisting of 2-Me-THF, MTBE, ethyl acetate, n-butanol, 1-propanol, and water.

In a first instance, Caprate 2 is crystallized from a solvent system comprising a solvent chosen from 1-propanol, MTBE, water, and mixtures thereof. In an embodiment, the solvent system comprising Compound 2 is aged to form Caprate 2. In particular, Caprate 2 is crystallized from a solvent system comprising MTBE, approximately 30-40% weight 1-propanol and 0.5-5% water.

In a subsequent instance, Caprate 2 is filtered and optionally dried. In an embodiment, Caprate 2 is dried resulting in a crystalline form of Formula I, such as Caprate 3. The drying can take place at room temperature, and/or at a relative humidity of about 50%.

In another instance, Caprate 9 is crystallized from a solvent system comprising a solvent chosen from 1-propanol, MTBE, water, and mixtures thereof. In an embodiment, the solvent system comprising Compound 2 is suspended to form Caprate 9. The solvent system can comprise multiple forms of Compound 2. In particular, Caprate 9 is crystallized from a solvent system comprising MTBE, approximately 5-40% weight 1-propanol and 0.5-5% water. In an embodiment, Caprate 9 is filtered and optionally dried. In an embodiment, Caprate 9 is dried resulting in a crystalline form of Formula I, such as Caprate 3. The drying can take place at room temperature, and/or at a relative humidity of about 50%.

### Definitions

Certain technical and scientific terms are specifically defined below. Unless specifically defined elsewhere in this document, all other technical and scientific terms used herein have the meaning commonly understood by one of ordinary skill in the art to which this disclosure relates. That is, terms used herein have their ordinary meaning, which is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates.

The terms used herein have their ordinary meaning and the meaning of such terms is independent at each occurrence thereof. That notwithstanding and except where stated otherwise, the following definitions apply throughout the specification and claims.

“FIG” (or “FIG.” or “Fig.” or “Fig” or “fig.” or “fig”) means “Figure” (or “figure”) and refers to the corresponding drawing.

Numerical values provided herein, and the use of the term “about,” may include variations of, for example, ±0.1%, ±0.2%, ±0.3%, ±0.4%, ±0.5%, 0.75, ±1%, ±2%, ±3%, ±4%, ±5%, and ±10% and their numerical equivalents. “About” when used to modify a numerically defined parameter (e.g., 2θ values of an X-ray powder diffraction pattern measured using CuKα radiation, or the chemical shift of a ¹³C or ¹⁵N as described herein) means that the parameter may vary by as much as 10% below or above the stated numerical value for that parameter; where appropriate, the stated parameter may be rounded to the nearest whole number. In addition, the term “or,” as used herein, denotes alternatives that may, where appropriate, be combined; that is, the term “or” includes each listed alternative separately as well as their combination.

Exemplary methods and materials are described herein, although methods and materials similar or equivalent to those described herein can also be used in the practice or testing of the present disclosure. The materials, methods, and examples are illustrative only and not intended to be limiting.

“Patient” includes both human and other animals.

“Mammal” includes humans and other mammalian animals.

“XPRD” refers to powder x-ray diffraction.

“Excipient” means an essentially inert substance used to give stability, form or consistency to a formulation.

“Diluent” is a type of excipient that primarily acts as a diluting agent. A diluent may act to decrease the viscosity of a fluid.

The term “composition” (or “pharmaceutical composition” or “pharmaceutically acceptable composition”) as used herein is intended to encompass a product comprising the specified ingredients in the specified amounts, as well as any product which results, directly or indirectly, from combining the specified ingredients in the specified amounts. The term is intended to encompass a product comprising active ingredient(s), and the inert ingredient(s), if any, that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the invention encompass any composition made by admixing a crystalline form of a compound of Formula I, as described herein, and a pharmaceutically acceptable carrier. By “pharmaceutically acceptable” it is meant the carrier, diluent or excipient must be compatible with the other ingredients of the formulation and not deleterious to the recipient thereof.

The term “composition” (or “pharmaceutical composition” or “pharmaceutically acceptable composition”) as used herein is also intended to encompass either the bulk composition and/or individual dosage units. (Such compositions and units can additionally comprise additional active ingredients as described herein.) The bulk composition and each individual dosage unit can contain fixed amounts of active agent(s). The bulk composition is material that has not yet been formed into individual dosage units. Non-limiting examples of dosage units include oral dosage units such as tablets, pills and the like. Similarly, the herein-described method of treating a patient by administering a pharmaceutical composition of the present invention is also intended to encompass administration of afore-said bulk composition and individual dosage units.

The term “caprate” as used herein is also known as “decanoate.”

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam—lactim pairs, enamine—imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution.

As used herein, the term “treating” or “treatment” refers to inhibiting or ameliorating a disease, condition or disorder in a subject who is experiencing or displaying the pathology or symptoms of the disease, condition or disorder. For example, inhibiting a disease, condition, or disorder refers to arresting further development of the pathology and/or symptoms of said disease, condition or disorder. Additionally, ameliorating a disease, condition or disorder, for example, refers to reversing the pathology and/or symptoms, such as decreasing the severity of the disease.

The term “prevent,” “preventing” or “prevention” as used herein, comprises the prevention of at least one symptom associated with or caused by the disease, condition or disorder being prevented.

As used herein, “subject” refers to an animal, preferably a mammal, and in particular a human or a non-human animal including livestock animals and domestic animals including, but not limited to, cattle, horses, sheep, swine, goats, rabbits, cats, dogs, and other mammals in need of treatment. In some embodiments, the subject is a human.

As used herein, the term “administration” and variants thereof (e.g., “administering”) in reference to the compound of Formula I means providing the compound to a subject in need of treatment. As used herein, “orally” and variants thereof (e.g., “oral”) refers to administration via the mouth, i.e., administration of the compound of Formula I through the mouth.

Administering of the compound of Formula I to the subject includes both self-administration and administration to the subject by another. The subject may be in need of, or desire, treatment for an existing disease or medical condition, or may be in need of or desire prophylactic treatment to prevent or reduce the risk of occurrence of the disease or medical condition. As used herein, a subject “in need” of treatment of an existing condition or of prophylactic treatment encompasses both a determination of need by a medical professional as well as the desire of a patient for such treatment.

### Processes

Provided herein are processes or methods of making a crystalline form of a compound of Formula I:

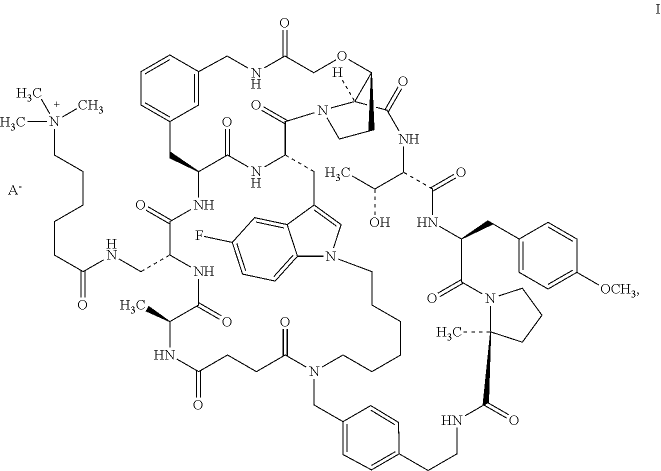

wherein A⁻ is a pharmaceutically acceptable anion;
formed by a process comprising adding an alcohol to a starting material,
wherein the starting material is selected from Compound 1, Compound 2, Compound 3, Compound 4, Compound 5, and Compound 6.

In an embodiment, the crystalline form of a compound of Formula I prepared by the above process is selected from Acetate 2, Acetate 3, Acetate 4, Acetate 5, Acetate 6, Caprate 2, Caprate 3, Caprate 4, Caprate 5, Caprate 6, Caprate 7, Caprate 8, Caprate 9, Caprate 10, Caprate 11, Caprate 12, Caprate 13, Caprate 14, D-Lactate 1, D-Lactate 2, Succinate 1, Succinate 2, L-Tartrate 1, L-Tartrate 2, Sulfate 1, and Sulfate 2.

In an embodiment of the process for preparing a crystalline form of a compound of Formula I, the alcohol is selected from ethanol, propanol, and butanol. In a further embodiment, the alcohol is ethanol. In an embodiment, the alcohol is propanol. In another embodiment, the alcohol is butanol. In still another embodiment, the alcohol is 1-propanol. In yet another embodiment, the alcohol is n-butanol.

In an embodiment, the process for preparing a crystalline form of a compound of Formula I comprises adding an organic solvent to the compound to form a slurry/solution. In a further embodiment, the process comprises aging the slurry/solution. In an embodiment, the process comprises aging the slurry/solution at a range of 0° C. to 40° C. In a further embodiment, the process comprises aging the slurry/solution at a range of 20 to 35° C.

In an embodiment, the process for preparing a crystalline form of a compound of Formula I comprises adding a mixture comprising an organic solvent and water. In a further embodiment, the process comprises adding the alcohol to the mixture. In another embodiment, the process comprises aging the mixture. In still another embodiment, the process comprises aging the mixture at 0° C. to 40° C. In a further embodiment, the process comprises filtering the mixture resulting in the formation of a wet cake. In an embodiment, the process comprises drying the wet cake. In a further embodiment, the process comprises drying the wet cake at 0° C. to 40° C.

In an embodiment, the process for preparing a crystalline form of a compound of Formula I comprises adding a mixture comprising an organic solvent. In a further embodiment, the alcohol is added in the mixture. In another embodiment, the process comprises aging the mixture. In still another embodiment, the process comprises aging the mixture at 0° C. to 40° C. In a further embodiment, the process comprises agitating the mixture. In an embodiment, the process comprises agitating the mixture at 0° C. to 20° C.

In an embodiment, the process for preparing a crystalline form of a compound of Formula I comprises washing the starting material with an organic solvent or the alcohol to form a wet cake. In another embodiment, the alcohol and the organic solvent are added together to form a mixture. In a further embodiment, the process further comprises drying the wet cake. In an embodiment, the process comprises drying the wet cake with nitrogen at 20° C. to 40° C.

In an embodiment, the process for preparing a crystalline form of a compound of Formula I further comprises exposing the crystalline form of the compound of Formula I to a relative humidity of about 5%, resulting in a second crystalline form of the compound of

Formula I. In another embodiment, the process for preparing a crystalline form of a compound of Formula I further comprises exposing the crystalline form of the compound of Formula I to a relative humidity of about 50%, resulting in a second crystalline form of the compound of Formula I.

In an embodiment of the process for preparing a crystalline form of a compound of Formula I, the organic solvent is selected from ethers, esters, and straight chained alkanes (C₃-C₁₀). In a further embodiment, the ether is 2-Me-THF. In yet another embodiment, the ether is MTBE. In an embodiment, the ester is ethyl acetate. In another embodiment, the alkane is heptane.

In a particular embodiment of the process for preparing a crystalline form of a compound of Formula I, the crystalline form of the compound of Formula I is selected from Acetate 2, Acetate 3, Acetate 4, Acetate 5, and Acetate 6 and the starting material is Compound 1 (the acetate salt of a compound of Formula I).

In a particular embodiment of the process for preparing a crystalline form of a compound of Formula I, the crystalline form of a compound of Formula I is selected from Caprate 2, Caprate 3, Caprate 4, Caprate 5, Caprate 6, Caprate 7, Caprate 8, Caprate 9, Caprate 10, Caprate 11, Caprate 12, Caprate 13, and Caprate 14 and the starting material is Compound 2 (the caprate salt of a compound of Formula I).

In a particular embodiment of the process for preparing a crystalline form of a compound of Formula I, the crystalline form of the compound of Formula I is selected from D-Lactate 1, Succinate 1, L-Tartrate 1, and Sulfate 1 and the starting material is Compound B (the bicarbonate salt of a compound of Formula I).

In a particular embodiment of the process for preparing a crystalline form of a compound of Formula I, the crystalline form of the compound of Formula I is D-Lactate 2 and the starting material is Compound 3 (the lactate salt of a compound of Formula I). In an embodiment, Compound 3 is D-Lactate 1.

In a particular embodiment of the process for preparing a crystalline form of a compound of Formula I, the crystalline form of the compound of Formula I is Succinate 2 and the starting material is Compound 4 (the succinate salt of a compound of Formula I). In an embodiment, Compound 4 is Succinate 1.

In a particular embodiment of the process for preparing a crystalline form of a compound of Formula I, the crystalline form of the compound of Formula I is L-Tartrate 2 and the starting material is Compound 5 (the tartrate salt of a compound of Formula I). In an embodiment, Compound 5 is L-Tartrate 1.

In a particular embodiment of the process for preparing a crystalline form of a compound of Formula I, the crystalline form of the compound of Formula I is Sulfate 2 and the starting material is Compound 6 (the sulfate salt of a compound of Formula I). In an embodiment, Compound 6 is Sulfate 1.

In another aspect, provided herein are processes or methods of making a crystalline form of a caprate salt of a compound of Formula I:

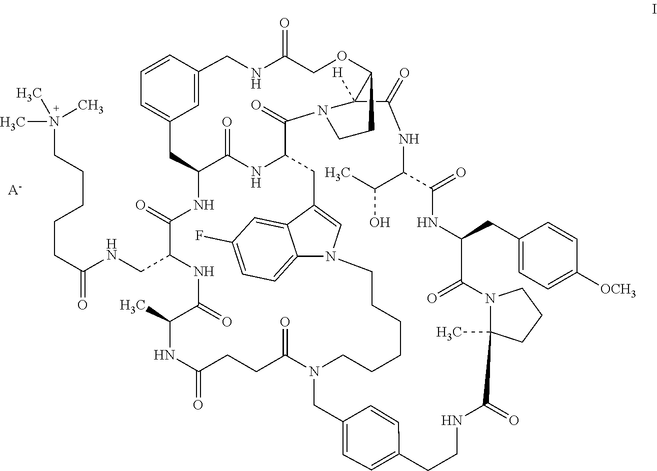

wherein A⁻ is caprate;
formed by a process comprising adding an alcohol to a form of a starting material,
wherein the starting material is Compound 2 (the caprate salt of a compound of Formula I).

In an embodiment of the process for preparing a crystalline form of a caprate salt of a compound of Formula I, the crystalline form prepared by the above process is selected from Caprate 2, Caprate 3, Caprate 4, Caprate 5, Caprate 6, Caprate 7, Caprate 8, Caprate 9, Caprate 10, Caprate 11, Caprate 12, Caprate 13, and Caprate 14.

In an embodiment, in the process for preparing a crystalline form of a caprate salt of a compound of Formula I, the alcohol is selected from ethanol, propanol, and butanol. In a further embodiment, the alcohol is ethanol. In an embodiment, the alcohol is propanol. In another embodiment, the alcohol is butanol. In still another embodiment, the alcohol is 1-propanol. In yet another embodiment, the alcohol is n-butanol.

In an embodiment, the process for preparing a crystalline form of a caprate salt of a compound of Formula I comprises adding an organic solvent to the starting material (Compound 2—the caprate salt of a compound of Formula I), to form a slurry/solution. In a further embodiment, the process comprises aging the slurry/solution. In an embodiment, the process comprises aging the slurry/solution at a range of 0° C. to 40° C. In a further embodiment, the process comprises aging the slurry/solution at a range of 20 to 35° C.

In an embodiment, the process for preparing a crystalline form of a caprate salt of a compound of Formula I comprises adding a mixture comprising an organic solvent and water. In a further embodiment, the process comprises adding the alcohol to the mixture. In another embodiment, the process comprises aging the mixture. In still another embodiment, the process comprises aging the mixture at 0° C. to 40° C. In a further embodiment, the process comprises filtering the mixture resulting in the formation of a wet cake. In an embodiment, the process comprises drying the wet cake. In a further embodiment, the process comprises drying the wet cake at 0° C. to 40° C.

In an embodiment, the process for preparing a crystalline form of a caprate salt of a compound of Formula I comprises adding a mixture comprising an organic solvent. In a further embodiment, the alcohol is added in the mixture. In another embodiment, the process comprises aging the mixture. In still another embodiment, the process comprises aging the mixture at 0° C. to 40° C. In a further embodiment, the process comprises agitating the mixture. In an embodiment, the process comprises agitating the mixture at 0° C. to 20° C.

In an embodiment, the process for preparing a crystalline form of a caprate salt of a compound of Formula I comprises washing the starting material (Compound 2—the caprate salt of a compound of Formula I), with an organic solvent or the alcohol to form a wet cake. In another embodiment, the alcohol and the organic solvent are added together to form a mixture. In a further embodiment, the process further comprises drying the wet cake. In an embodiment, the process comprises drying the wet cake with nitrogen at 20° C. to 40° C.

In an embodiment, the process for preparing a crystalline form of a caprate salt of a compound of Formula I further comprises exposing the crystalline form of the caprate salt of the compound of Formula I to a relative humidity of about 5%, resulting in a second crystalline form of the caprate salt of the compound of Formula I. In an embodiment, the process for preparing a crystalline form of a caprate salt of a compound of Formula I further comprises exposing the crystalline form of the caprate salt of the compound of Formula I to a relative humidity of about 50%, resulting in a second crystalline form of the caprate salt of the compound of Formula I. In an embodiment, the organic solvent of the above process is selected from ether, ester, and a straight chained alkane (C₃-C₁₀). In a further embodiment, the ether is 2-Me-THF. In yet another embodiment, the ether is MTBE. In an embodiment, the ester is ethyl acetate.

In an embodiment of the process for preparing a crystalline form of the caprate salt of a compound of Formula I, the starting material is selected from Caprate 1, Caprate 2, Caprate 3, Caprate 4, Caprate 5, Caprate 6, Caprate 7, Caprate 8, Caprate 9, Caprate 10, Caprate 11, Caprate 12, Caprate 13, and Caprate 14.

In an embodiment, the crystalline form of a caprate salt of a compound of Formula I is Caprate 4, and the starting material is Caprate 1.

In an embodiment, the crystalline form of a caprate salt of compound of Formula I is Caprate 5, and the starting material is Caprate 4.

In an embodiment, the crystalline form of a caprate salt of compound of Formula I is Caprate 2, and the starting material is Compound 2. In another embodiment, the crystalline form of a caprate salt of compound of Formula I is Caprate 2, and the starting material is Caprate 5. In yet another embodiment, the crystalline form of a caprate salt of compound of Formula I is Caprate 2, and the starting material is Compound A or Compound B. In a further embodiment, the process comprises adding a mixture comprising an organic solvent and water. In another further embodiment, the process comprises adding the alcohol to the mixture. In an embodiment, the organic solvent is an ether. In still another embodiment, the ether is MTBE. In yet an embodiment, the alcohol is propanol. In a further embodiment, the alcohol is 1-propanol. In an embodiment, the mixture is comprised of MTBE, approximately 30-40% weight 1-propanol and 0.5-5% water. In a further embodiment, the mixture is comprised of MTBE, approximately 39% weight 1-propanol and 1% water. In another embodiment, the process comprises aging the mixture. In a further embodiment, the process comprises aging the mixture at 20° C. to 30° C.

In an embodiment, the crystalline form of a compound of a caprate salt of Formula I is Caprate 3, and the starting material is Caprate 2 or Caprate 9. In a further embodiment, the process comprises filtering and optionally drying Caprate 2. In an embodiment, the process comprises drying Caprate 2 to form Caprate 3. In a further embodiment, the process comprises drying Caprate 2 at 20° C. to 30° C. to form Caprate 3. The drying can take place at room temperature, and/or at a relative humidity of about 50%.

As also described above, Caprate 3 can be formed by drying a composition comprising Caprate 9. The drying can take place at room temperature, and/or at a relative humidity of about 50%.

In an embodiment, the crystalline form of a caprate salt of a compound of Formula I is Caprate 4, and the starting material is Caprate 3.

In an embodiment, the crystalline form of a caprate salt of a compound of Formula I is Caprate 6, and the starting material is Caprate 1.

In an embodiment, the crystalline form of a caprate salt of a compound of Formula I is Caprate 7, and the starting material is Caprate 6.

In an embodiment, the crystalline form of a caprate salt of a compound of Formula I is Caprate 8, and the starting material is Caprate 4.

In an embodiment, the crystalline form of a caprate salt of a compound of Formula I is selected from Caprate 9 and Caprate 12 and the starting material is a mixture of Caprate 3, Caprate 5, and Caprate 8.

In an embodiment, the process for preparing a crystalline form of a caprate salt of a compound of Formula I further comprises exposing the first crystalline form of a compound of Formula I selected from Caprate 3 and Caprate 7 to a relative humidity of about 5% and resulting in a second crystalline form of a caprate salt of a compound of Formula I selected from Caprate 10 and Caprate 11. In a further embodiment, the first crystalline form of a caprate salt of a compound of Formula I is Caprate 3 and the second crystalline form of a caprate salt of a compound of Formula I is Caprate 10. In another embodiment, the first crystalline form of a caprate salt of a compound of Formula I is Caprate 7 and the second crystalline form of a caprate salt of a compound of Formula I is Caprate 11.

In an embodiment, the crystalline form of a caprate salt of a compound of Formula I is selected from Caprate 13 and Caprate 14 and the starting material is Caprate 3.

In an embodiment, the process for preparing a crystalline form of a caprate salt of a compound of Formula I comprises exposing Caprate 3 to a vapor comprising the alcohol and the crystalline form of a compound of Formula I is Caprate 13. In another embodiment, the process comprises exposing Caprate 3 to a vapor comprising organic solvent and the alcohol, wherein the crystalline form of a compound of Formula I is Caprate 13. In a further embodiment, the organic solvent is MTBE.

Also provided herein are processes or methods of making a crystalline form of a compound of Formula I:

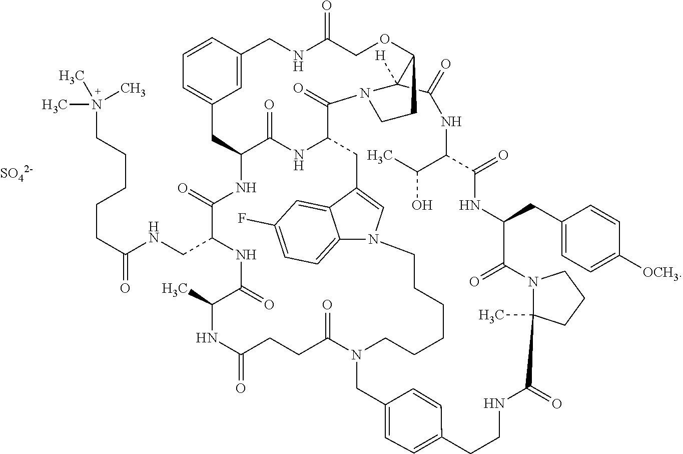

wherein A⁻ is a pharmaceutically acceptable anion;
formed by a process comprising adding an alcohol to a starting material,
wherein the starting material is Compound A (the chloride salt of a compound of Formula I) or Compound B (the bicarbonate salt of a compound of Formula I).

In an embodiment, the process of making a crystalline compound of Formula I comprises an ion exchange.

In an embodiment, the process of making a crystalline compound of Formula I comprises an ion exchange, wherein the staring material is Compound A (the chloride salt of a compound of Formula I) and the ion exchange comprises an ion exchange resin charged with A⁻.

In another embodiment, Compound B (the bicarbonate salt of a compound of Formula I) is formed by a process comprising an ion exchange, wherein the initial material is Compound A (the chloride salt of a compound of Formula I) and the ion exchange forming Compound B comprises:
(1) an ion exchange resin charged with bicarbonate; or
(2) a liquid-liquid extraction with an organic solvent and aqueous bicarbonate.

In a further embodiment, the process of making a crystalline compound of Formula I comprises ion exchange, wherein the starting material is Compound B (the bicarbonate salt of a compound of Formula I) and the ion exchange comprises adding an acid comprising the pharmaceutically acceptable anion.

In an embodiment, the crystalline form of a compound of Formula I prepared by the above process is selected from Acetate 2, Acetate 3, Acetate 4, Acetate 5, Acetate 6, Caprate 2, Caprate 3, Caprate 4, Caprate 5, Caprate 6, Caprate 7, Caprate 8, Caprate 9, Caprate 10, Caprate 11, Caprate 12, Caprate 13, Caprate 14, D-Lactate 1, D-Lactate 2, Succinate 1, Succinate 2, L-Tartrate 1, L-Tartrate 2, Sulfate 1, and Sulfate 2.

In an embodiment of the process for preparing a crystalline form of a compound of Formula I, the alcohol is selected from ethanol, propanol, and butanol. In a further embodiment, the alcohol is ethanol. In an embodiment, the alcohol is propanol. In another embodiment, the alcohol is butanol. In still another embodiment, the alcohol is 1-propanol. In yet another embodiment, the alcohol is n-butanol.

In an embodiment, the process for preparing a crystalline form of a compound of Formula I comprises adding an organic solvent to the compound to form a slurry/solution. In a further embodiment, the process comprises aging the slurry/solution. In an embodiment, the process comprises aging the slurry/solution at a range of about −10° C. to about 40° C. In a further embodiment, the process comprises aging the slurry/solution at a range of about 20 to about 35° C. In another embodiment, the process comprises aging the slurry/solution at a range of about-10 to about 0° C.

In an embodiment, the process for preparing a crystalline form of a compound of Formula I comprises adding a mixture comprising an organic solvent and water. In a further embodiment, the process comprises adding the alcohol to the mixture. In another embodiment, the process comprises aging the mixture. In still another embodiment, the process comprises aging the mixture at about −10° C. to about 40° C. In a further embodiment, the process comprises filtering the mixture resulting in the formation of a wet cake. In an embodiment, the process comprises drying the wet cake. In a further embodiment, the process comprises drying the wet cake at about 0° C. to about 40° C.

In an embodiment, the process for preparing a crystalline form of a compound of Formula I comprises adding a mixture comprising an organic solvent. In a further embodiment, the alcohol is added in the mixture. In another embodiment, the process comprises aging the mixture. In still another embodiment, the process comprises aging the mixture at about −10° C. to about 40° C. In a further embodiment, the process comprises agitating the mixture. In an embodiment, the process comprises agitating the mixture at about 0° C. to about 20° C.

In an embodiment, the process for preparing a crystalline form of a compound of Formula I comprises washing the starting material with an organic solvent or the alcohol to form a wet cake. In another embodiment, the alcohol and the organic solvent are added together to form a mixture. In a further embodiment, the process further comprises drying the wet cake. In an embodiment, the process comprises drying the wet cake with nitrogen at about 20° C. to about 40° C.

In an embodiment, the process for preparing a crystalline form of a compound of Formula I further comprises exposing the crystalline form of the compound of Formula I to a relative humidity of about 5% to about 50%, resulting in a second crystalline form of the compound of Formula I. In an embodiment, the relative humidity is about 5%. In another embodiment, the relative humidity is about 50%.

In an embodiment of the process for preparing a crystalline form of a compound of Formula I, the organic solvent is selected from ethers, esters, and straight chained alkanes (C₃-C₁₀). In a further embodiment, the ether is 2-Me-THF. In yet another embodiment, the ether is MTBE. In an embodiment, the ester is ethyl acetate. In another embodiment, the alkane is heptane.

In a particular embodiment of the process for preparing a crystalline form of a compound of Formula I, wherein the crystalline form of the compound of Formula I is selected from Acetate 2, Acetate 3, Acetate 4, Acetate 5, and Acetate 6 and the starting material is Compound A (the chloride salt of a compound of Formula I). In another embodiment of the process for preparing a crystalline form of a compound of Formula I, the crystalline form of the compound of Formula I is selected from Acetate 2, Acetate 3, Acetate 4, Acetate 5, and Acetate 6 and the starting material is Compound B (the bicarbonate salt of a compound of Formula I).

In a particular embodiment of the process for preparing a crystalline form of a compound of Formula I, wherein the crystalline form of a compound of Formula I is selected from Caprate 2, Caprate 3, Caprate 4, Caprate 5, Caprate 6, Caprate 7, Caprate 8, Caprate 9, Caprate 10, Caprate 11, Caprate 12, Caprate 13, and Caprate 14 and the starting material is Compound A (the chloride salt of a compound of Formula I). In another embodiment of the process for preparing a crystalline form of a compound of Formula I, the crystalline form of a compound of Formula I is selected from Caprate 2, Caprate 3, Caprate 4, Caprate 5, Caprate 6, Caprate 7, Caprate 8, Caprate 9, Caprate 10, Caprate 11, Caprate 12, Caprate 13, and Caprate 14 and the starting material is Compound B (the bicarbonate salt of a compound of Formula I).

In a particular embodiment of the process for preparing a crystalline form of a compound of Formula I, wherein the crystalline form of the compound of Formula I is selected from D-Lactate 1, Succinate 1, L-Tartrate 1, and Sulfate 1 and the starting material is Compound B (the bicarbonate salt of a compound of Formula I).

### Method of Treating

In another aspect provided herein are methods of employing PCSK9-specific antagonist compounds (e.g., compounds of Formula I) described herein for antagonizing PCSK9 function; said methods of which are further described below. Use of the term “antagonizing” throughout the present application refers to providing to the affected tissue(s) a substance which opposes the action of, inhibits, counteracts, neutralizes or curtails one or more functions of PCSK9 in the affected tissues. Inhibition or antagonism of one or more of PCSK9-associated functional properties can be readily determined according to methodologies known to the art (see, e.g., Barak & Webb, 1981 J. Cell Biol. 90:595-604; Stephan & Yurachek, 1993 J. Lipid Res. 34:325330; and McNamara et al., 2006 Clinica Chimica Acta 369:158-167) as well as those described herein. Inhibition or antagonism will effectuate a decrease in PCSK9 activity relative to that seen in the absence of the antagonist or, for example, that seen relative to the activity observed when a control antagonist of irrelevant specificity is present. Preferably, a PCSK9-specific antagonist in accordance with the present invention antagonizes PCSK9 functioning to the point that there is a decrease of at least 10%, of the measured parameter including but not limited to the activities disclosed herein, and more preferably, a decrease of at least 20%, 30%, 40%, 50%, 60%, 70%, 80%, 90% and 95% of the measured parameter. Such inhibition/antagonism of PCSK9 functioning is particularly effective in those instances where PCSK9 functioning is contributing at least in part to a particular phenotype, disease, disorder or condition which is negatively impacting the subject.

In one aspect, the present invention provides a method for antagonizing the activity of PCSK9, which comprises contacting a cell, population of cells or tissue sample capable of being affected by PCSK9 (i.e., which expresses and/or comprises LDL receptors) with a PCSK9-specific antagonist disclosed herein (e.g., compounds of Formula I) under conditions that allow said antagonist to bind to PCSK9 when present and inhibit PCSK9's inhibition of cellular LDL uptake. In some embodiments of the present invention include such methods wherein the cell is a human cell. Additional embodiments of the present invention include such methods wherein the cell is a murine cell.

In one aspect, the present invention provides a method for antagonizing the activity of PCSK9 in a subject, which comprises administering to the subject a therapeutically effective amount of a PCSK9-specific antagonist of the present invention. In some embodiments, the methods for antagonizing PCSK9 function are for the treatment, as defined herein, of a PCSK9-associated disease, disorder or condition or, alternatively, for providing therapy in a disease, disorder or condition that could benefit from the effects of a PCSK9 antagonist.

The present invention, thus, contemplates the use of PCSK9-specific antagonists described herein in various methods of treatment where antagonizing PCSK9 function is desirable. As used herein, the term “method of treatment” relates to a course of action resulting in a change in at least one symptom of a disease state which can be prophylactic or therapeutic in nature. In some embodiments, the present invention relates to a method of treatment for a condition associated with and/or attributed to PCSK9 activity, or a condition where the functioning of PCSK9 is contraindicated for a particular subject, the method comprising administering to the subject a therapeutically effective amount of a PCSK9-antagonist compound of Formula I, or pharmaceutically acceptable salt thereof. In some embodiments, the condition may be atherosclerosis, hypercholesterolemia, peripheral arterial disease, cerebrovascular disease, coronary heart disease, metabolic syndrome, acute coronary syndrome or related cardiovascular disease and cardiometabolic conditions, or may be a disease state or condition in which PCSK9 activity is contraindicated.

In an aspect, provided herein is the use of a crystalline form of a compound of Formula I as an active ingredient in a medicament for treating hypercholesterolemia in a subject.

In an embodiment, provided herein in the use of a pharmaceutical composition comprising a crystalline form of a compound of Formula I as a medicament for treating hypercholesterolemia in a subject.

In another embodiment, provided herein is the use of a crystalline form of a compound of Formula I as an active ingredient in a medicament for reducing LDL-C in a subject.

In yet another embodiment, provided herein is the use of a crystalline form of a compound of Formula I as an active ingredient in a medicament for treating atherosclerotic cardiovascular disease in a subject.

In an aspect, provided herein is a method of treating hypercholesterolemia, comprising administering to a patient in need thereof a therapeutically effective amount of a compound of a crystalline form of a compound of Formula I.

In an aspect, provided herein is the use of a crystalline form of a compound of Formula I as an active ingredient in a medicament for treating peripheral arterial disease in a subject.

In an embodiment, provided herein in the use of a pharmaceutical composition comprising a crystalline form of a compound of Formula I as a medicament for treating peripheral arterial disease in a subject.

In an aspect, provided herein is the use of Caprate 3 as an active ingredient in a medicament for treating hypercholesterolemia in a subject.

In an embodiment, provided herein is a pharmaceutical composition comprising Caprate 3 as a medicament for treating hypercholesterolemia in a subject.

In another embodiment, provided herein is the use of Caprate 3 as an active ingredient in a medicament for reducing LDL-C in a subject.

In yet another embodiment, provided herein is the use of Caprate 3 as an active ingredient in a medicament for treating atherosclerotic cardiovascular disease in a subject.

In an aspect, provided herein is a method of treating hypercholesterolemia, comprising administering to a patient in need thereof a therapeutically effective amount of Caprate 3.

In an aspect, provided herein is the use of Caprate 3 as an active ingredient in a medicament for treating peripheral arterial disease in a subject.

In an embodiment, provided herein is a pharmaceutical composition comprising Caprate 3 as a medicament for treating peripheral arterial disease in a subject.

### Pharmaceutical Compositions

Methods of treatment in accordance with the present invention comprise administering to an individual a therapeutically (or prophylactically) effective amount of a PCSK9-specific antagonist of the present invention. Use of the terms “therapeutically effective” or “prophylactically effective” in reference to an amount refers to the amount necessary at the intended dosage to achieve the desired therapeutic and/or prophylactic effect for the period of time desired. The desired effect may be, for example, the alleviation, amelioration, reduction or cessation of at least one symptom associated with the treated condition. These amounts will vary, as the skilled artisan will appreciate, according to various factors, including but not limited to the disease state, age, sex, and weight of the individual, and the ability of the PCSK9-specific antagonist to elicit the desired effect in the individual. The response may be documented by in vitro assay, in vivo non-human animal studies, and/or further supported from clinical trials.

In some embodiments it is preferred to administer a PCSK9 antagonist compound of the invention in the form of a pharmaceutical composition as described herein.

Dosing of antagonist therapeutics is well within the realm of the skilled artisan, see, e.g., Lederman et al., 1991 Int. J. Cancer 47:659-664; Bagshawe et al., 1991 Antibody, Immunoconjugates and Radiopharmaceuticals 4:915-922, and will vary based on a number of factors, for example, but not limited to, those mentioned above, including the condition of the patient, the area being treated, the route of administration, and the treatment desired, for example, prophylaxis or acute treatment and the like. A physician or veterinarian of ordinary skill can readily determine and prescribe the effective therapeutic amount of the antagonist.

The subject may be in need of, or desire, treatment for an existing disease or medical condition. As used herein, the subject “in need” of treatment of an existing condition encompasses both a determination of need by a medical professional as well as the desire of the subject for such treatment. The term “subject,” as used herein, refers to a mammal, a plant, a lower animal, or a cell culture. In one embodiment, a subject is a human or other animal patient in need of treatment. When a compound or a salt thereof is provided in combination with one or more other active agents, “administration” and its variants are each understood to include provision of the compound or its salt and the other agents contemporaneously or simultaneously or over a course of separate administrations over a period of time. When the agents of a combination are administered at the same time, they can be administered together in a single composition or they can be administered separately. It is understood that a “combination” of active agents can be a single composition containing all of the active agents or multiple compositions each containing one or more of the active agents. In the case of two active agents a combination can be either a single composition comprising both agents or two separate compositions each comprising one of the agents; in the case of three active agents a combination can be either a single composition comprising all three agents, three separate compositions each comprising one of the agents, or two compositions one of which comprises two of the agents and the other comprises the third agent; and so forth.

The compositions and combinations of the present invention are suitably administered in effective amounts. The term “effective amount” means the amount of active compound sufficient to antagonize PCSK9 and thereby elicit the response being sought (i.e., induce a therapeutic response in the treatment or management of conditions associated with or impacted by PCSK9 function, including, but not limited to atherosclerosis, hypercholesterolemia, peripheral arterial disease, cerebrovascular disease, coronary heart disease, metabolic syndrome, acute coronary syndrome, and related cardiovascular disease and cardiometabolic conditions in an animal or human).

The actual dosage employed may be varied depending upon the requirements of the patient and the severity of the condition being treated. Determination of the proper dosage regimen for a particular situation is within the skill in the art, for example, as described in the standard literature, for example, as described in the “Physicians' Desk Reference” (PDR), e.g., 1996 edition (Medical Economics Company, Montvale, NJ 07645-1742, USA), the Physician's Desk Reference, 56th Edition, 2002 (published by Medical Economics company, Inc. Montvale, NJ 07645-1742), or the Physician's Desk Reference, 57th Edition, 2003 (published by Thompson PDR, Montvale, NJ 07645-1742); the disclosures of which is incorporated herein by reference thereto. For convenience, the total daily dosage may be divided and administered in portions during the day as required or delivered continuously.

The PCSK9-specific antagonist may be administered to an individual by any route of administration appreciated in the art, including but not limited to oral administration, administration by injection (specific embodiments of which include intravenous, subcutaneous, intraperitoneal or intramuscular injection), or administration by inhalation, intranasal, or topical administration, either alone or in combination with other agents designed to assist in the treatment of the individual. The PCSK9-specific antagonist may also be administered by injection devices, injector pens, needleless devices; and subcutaneous patch delivery systems. The route of administration should be determined based on a number of considerations appreciated by the skilled artisan including, but not limited to, the desired physiochemical characteristics of the treatment.

### Doses and Formulation

Also provided herein is a method of treating hypercholesterolemia in a subject in need of such treatment, comprising orally administering to the subject an amount of a crystalline form of a compound of Formula I:

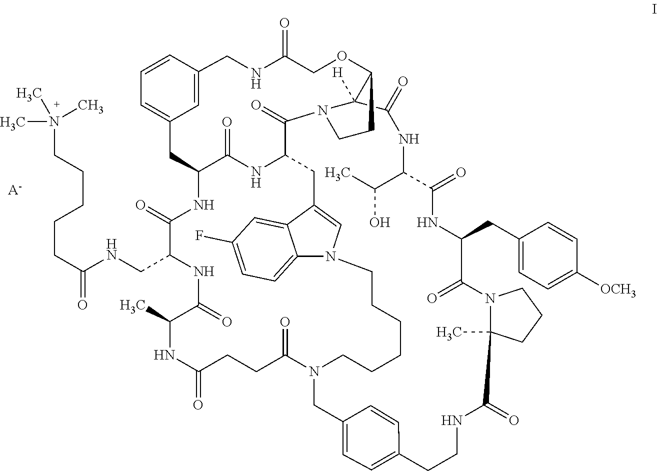

wherein A⁻ is selected from a pharmaceutically acceptable anion, and wherein the amount administered is from about 5 mg to about 300 mg of the compound of Formula I.

The dosage regimen is selected in accordance with a variety of factors including type, species, age, weight, sex, and medical condition of the patient; the severity of the condition to be treated; the route of administration; and the renal and hepatic function of the patient. An ordinarily skilled physician, veterinarian, or clinician can readily determine and prescribe the effective amount of the drug required to prevent, counter, or arrest the progress of the condition.

The forms of the present disclosure may be formulated and administered in solid dosage forms, such as tablets, pills, capsules, powders, or granules, which are intended for oral administration. Formulation of the compositions according to the disclosure can conveniently be by methods known from the art, for example, as described in Remington's Pharmaceutical Sciences, 18th ed., 1990, and Remington: The Science and Practice of Pharmacy, 22^{nd} ed., 2012. Furthermore, the forms of the present disclosure may be formulated and administered in sterile solutions for enteral (oral), parenteral, intravenous, or intramuscular administration.

In the methods of the present disclosure, the forms described herein may be formulated as the active pharmaceutical ingredient, and may be administered in admixture with suitable pharmaceutical diluents, excipients, or carriers (collectively referred to herein as “carrier” materials) suitably selected with respect to the intended form of administration and consistent with conventional pharmaceutical practices, that is, oral tablets, oral capsules, oral suspensions, oral formulations, or sterile solutions for parenteral, intravenous, or intramuscular administration.

For instance, for oral administration in the form of a tablet or capsule, the form described herein can be combined with an oral, non-toxic, pharmaceutically acceptable, inert carrier (such as lactose, starch, sucrose, glucose, methyl cellulose, magnesium stearate, dicalcium phosphate, calcium sulfate, mannitol, sorbitol, and the like). For parenteral, intravenous, or intramuscular administration in the form of a sterile solution, the form described herein may be combined with suitable excipients and non-toxic, pharmaceutically acceptable, inert carrier into a formulation that may be provided as a prepared dosage form in a pre-filled injection apparatus, as a lyophilized formulation to be reconstituted for injection, or as a sterile liquid to be diluted for injection.

In an embodiment, the amount administered to the subject is from about 5 mg to about 300 mg of the crystalline form of the compound of Formula I. Whole and half integers between 5 and 300 mg are included in this invention. In an embodiment, the amount administered is from about 10 mg to about 300 mg of the crystalline form of the compound of Formula I. In an embodiment, the amount administered is about 10 mg or about 20 mg or about 22 mg of the crystalline form of the compound of Formula I. In an embodiment, the amount administered is about 5 mg, about 6 mg, about 10 mg, about 12 mg, about 15 mg, about 18 mg, about 20 mg, about 22 mg, about 24, about 25, about 30 mg, about 35 mg, about 40 mg, or about 100 mg of the crystalline form of the compound of Formula I. In an embodiment, the amount administered is about 10 mg, about 12 mg, about 15 mg, about 18 mg, about 20 mg, about 22 mg, about 24 mg, about 25 mg, about 30 mg, or about 40 mg of the crystalline form of the compound of Formula I. In an embodiment, the amount administered is about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 26.5, about 27, about 27.5, about 28, about 28.5, about 29, about 29.5, or about 30 mg of the crystalline form of the compound of Formula I. In an embodiment, the amount administered is a daily dose of about 5 mg to about 300 mg. In an embodiment, the amount administered is a daily dose of about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 26.5, about 27, about 27.5, about 28, about 28.5, about 29, about 29.5, about 30 mg, about 35 mg or about 40 mg of the crystalline form of the compound of Formula I. In an embodiment, the amount administered is a daily dose of about 5, about 6, about 10, about 12, about 15, about 18, about 20, about 22, about 22.5, about 24, about 25, or about 30 mg of the crystalline form of the compound of Formula I. In an embodiment, the amount administered is a daily dose of about 10, about 12, about 15, about 18, about 20, about 22, about 24, about 25, or about 30 mg of the crystalline form of the compound of Formula I.

In an embodiment, the amount of Formula I administered to the subject is from about 10 mg to about 40 mg of the crystalline form of the compound of Formula I. In an embodiment, the amount of Formula I administered to the subject is from about 10 mg to about 30 mg of the crystalline form of the compound of Formula I. In another embodiment, the amount administered to the subject is from about 12 mg to about 27 mg of the crystalline form of the compound of Formula I. In still another embodiment, the amount administered to the subject is from about 15 mg to about 25 mg of the crystalline form of the compound of Formula I. In an embodiment, the amount administered to the subject is from about 10 mg to about 22.5 mg of the crystalline form of the compound of Formula I. In an embodiment, the amount administered to the subject is from about 10 mg to about 20 mg of the crystalline form of the compound of Formula I. In yet another embodiment, the amount administered to the subject is from about 15 mg to about 20 mg of the crystalline form of the compound of Formula I.

In an embodiment, the amount administered to the subject is from about 10 mg to about 30 mg of Caprate 3. In another embodiment, the amount administered to the subject is from about 12 mg to about 27 mg of Caprate 3. In still another embodiment, the amount administered to the subject is from about 15 mg to about 25 mg of Caprate 3. In an embodiment, the amount administered to the subject is from about 10 mg to about 22 mg of Caprate 3. In an embodiment, the amount administered to the subject is from about 10 mg to about 20 mg of Caprate 3. In yet another embodiment, the amount administered to the subject is from about 15 mg to about 22 mg of Caprate 3. In an embodiment, the amount is a daily dose of about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 26.5, about 27, about 27.5, about 28, about 28.5, about 29, about 29.5, or about 30 mg of Caprate 3. In an embodiment, the amount is a daily dose of about 15, about 15.5, about 16, about 16.5, about 17, about 17.5, about 18, about 18.5, about 19, about 19.5, about 20, about 20.5, about 21, about 21.5, about 22 mg of Caprate 3.

In yet another embodiment, the amount administered to the subject in need is about 15 mg, about 17.5 mg, 18 mg, about 20 mg or about 22 mg of Caprate 3. In a further embodiment, the amount administered to the subject in need is about 20 mg or about 22 mg of Caprate 3. In a further embodiment, the amount administered to the subject in need is about 20 mg of Caprate 3. In a further embodiment, the amount administered to the subject in need is about 22 mg of Caprate 3.

It is to be understood that the administration of a particular dose of the crystalline form of the compound of Formula I will correspond to the administration of the corresponding free form of the compound of Formula I. For example, the administration of about 22 mg of Caprate 3 corresponds to administration of about 20 mg of the corresponding free form of the compound of Formula I.

In an embodiment, orally administering comprises administering a single oral dosage form comprising the amount of the crystalline form of the compound of Formula I. In an embodiment, orally administering comprises administering more than one or multiple oral dosage forms, each comprising the amount of the crystalline form of the compound of Formula I or a portion thereof. In an embodiment, orally administering comprises administering a single oral dosage form comprising the amount of the crystalline form of the compound of Formula I once daily. In an embodiment, orally administering comprises administering more than one or multiple oral dosage forms, each comprising the amount of the crystalline form of the compound of Formula I or a portion thereof, once daily. In an embodiment, orally administering comprises administering a single oral dosage form comprising the amount of the crystalline form of the compound of Formula I more than once daily, e.g., twice, three times or four times daily. In an embodiment, orally administering comprises administering more than one or multiple oral dosage forms, each comprising the amount of the crystalline form of the compound of Formula I or a portion thereof, more than once daily, e.g., twice, three times or four times daily. The oral dosage form may be administered with or without fasting, i.e., with or without food. In an embodiment, the subject in need of treatment fasts approximately 30 minutes before the administration of the crystalline form of the compound of Formula I.

In an embodiment, a single oral dosage form is administered once daily for at least 14 days. In an embodiment, a single oral dosage form is administered once daily for 14 days. In an embodiment, a single oral dosage form is administered once daily for as long as the subject is in need of the treatment.

As used herein, an “oral dosage form” refers to a pharmaceutical formulation, comprising the crystalline form of the compound of Formula I and at least one pharmaceutically acceptable excipient, that is suitable for administration through the mouth of the subject. As used herein, the terms “oral dosage form” and “pharmaceutical composition” are intended to encompass both the combination of the specified ingredients in the specified amounts, and any product which results, directly or indirectly, from combination of the specified ingredients in the specified amounts. An oral dosage form may include the entire amount of the crystalline form of the compound of Formula I, e.g., about 5 mg to about 300 mg, which may or may not be a daily dose. An oral dosage form may include a portion of a daily dose of the crystalline form of the compound of Formula I.

The oral dosage forms according to the disclosure can be solid, semi-solid or liquid. Such oral dosage forms include, but are not limited to, powders, dispersible granules, mini-tablets, and beads (which can be used, for example, for tableting, encapsulation, or direct administration), pills, tablets, lacquered tablets, sugar-coated tablets, hard and soft capsules including gelatin capsules, lozenges, rapidly dissolving tablets, aqueous, alcoholic or oily solutions, gels, syrups, emulsions or suspensions. The oral dosage forms according to the disclosure may comprise additionally one or more coatings which modify release properties, for example, coatings which impart delayed release or formulations which have extended release properties. Also included in the present disclosure are formulations which are intended to be converted, shortly before use, to a suspension or a solution; examples include, but are not limited to, freeze-dried formulations and liquid formulations adsorbed into a solid absorbent medium. In an embodiment, the oral dosage form is a liquid-filled capsule, e.g., a hard gelatin capsule filled with the crystalline form of the compound of Formula I in a combination of Labrasol® and propylene glycol in, e.g., a 2:1 ratio. In an embodiment, the oral dosage form is a liquid-filled capsule, e.g., a hard gelatin capsule filled with the crystalline form of the compound of Formula I in a combination of Labrasol® and propylene glycol in, e.g., a 2:1 ratio, over-encapsulated with an enteric capsule, e.g., an HPMC Vcaps® Enteric capsule (Capsugel®, Lonza). In an embodiment, the oral dosage form is a suspension, e.g., the crystalline form of the compound of Formula I suspended in a combination of OraBlend SF and propylene glycol in, e.g., a 2:1 ratio. In an embodiment, the oral dosage form is a dry-filled enteric coated capsule, e.g., a dry-filled HPMC Vcaps® Enteric capsule (Capsugel®, Lonza). In an embodiment, the oral dosage form is a tablet. In a further embodiment, the oral dosage form is tablet which is film-coated.

In an embodiment, a pharmaceutical composition provided herein contains a diluent selected from a polyethylene glycol (of varying molecular weights above 300), microcrystalline cellulose, mannitol, starch, dicalcium phosphate, calcium carbonate, sodium carbonate, lactose or combinations thereof. In an embodiment, the pharmaceutical composition provided herein contains a diluent selected from PEG300, macrogol (PEG4000)), microcrystalline cellulose, mannitol, lactose, or combinations thereof. In a further embodiment, the diluent is selected from PEG300, macrogol (PEG4000), microcrystalline cellulose or lactose.

In an embodiment, a pharmaceutical composition provided herein contains a binder selected from hydroxypropyl cellulose, hydroxypropyl methyl cellulose or polyvinyl pyrrolidone. In a further embodiment, the binder is hydroxypropyl cellulose. In an embodiment, the binder is used in wet granulation (high shear, twin screw or fluid bed granulation)

In an embodiment, a pharmaceutical composition of provided herein contains a disintegrant selected from croscarmellose sodium, crospovidone, or sodium starch glycolate. In a further embodiment, the disintegrant is croscarmellose sodium. In an embodiment, a pharmaceutical composition of the instant invention contains a glidant selected from silicon dioxide, starch, talc, magnesium stearate, or tricalcium phosphate. In a further embodiment, the glidant is selected from silicon dioxide or tricalcium phosphate. In an embodiment, a pharmaceutical composition of the instant invention contains a lubricant selected from magnesium stearate or sodium stearyl fumerate or both. In an embodiment, a pharmaceutical composition of the instant invention contains a solubilizing agent selected from propylene gylcol, polysorbate 80, sorbitol, cremophor EL, castor oil, corn oil, cottonseed oil, safflower oil, sesame oil, soybean oil, peppermint oil, olive oil, miglyol, glycerin or combinations thereof. In a further embodiment, the solubilizing agent is a propylene glycol.

In an embodiment, an oral dosage form further comprises a permeation enhancer. As used herein, a “permeation enhancer” refers to a pharmaceutically acceptable excipient which improves the absorption of an active agent, e.g., the crystalline form of the compound of Formula I, from the gastrointestinal tract. Permeation enhancers afford the absorption of cell-impermeable compounds by promoting size-limited passage through tight junctions between intestinal epithelial cells. (D. J. Drucker, Advances in oral peptide therapeutics, Nat Rev Drug Discov, 19, pp 277-289 (2020). Suitable permeation enhancers include, without limitation, sodium caprate, Labrasol®, salcaprozate sodium (SNAC) and combinations thereof. Labrasol® is also known as Caprylocaproyl macrogol-8 glycerides and is manufactured by Gattefosse, Saint Priest, Lyon, France. In an embodiment, an oral dosage form comprises Labrasol®. In an embodiment, an oral dosage form comprises sodium caprate. When present in an oral dosage form, an amount of up to 1800 mg, an amount of up to about 720 mg, an amount of up to about 540 mg, an amount of up to about 360 mg, an amount ranging from about 90 mg to about 360 mg, an amount ranging from about 180 to about 360 mg, or an amount of 90 mg, 180 mg or 360 mg of permeation enhancer is used. In an embodiment provided herein, an oral dosage form comprises an amount of up to about 360 mg, an amount ranging from about 90 mg to about 360 mg, an amount ranging from about 180 to about 360 mg, or an amount of 90 mg, 180 mg or 360 mg of a permeation enhancer. In an embodiment, the oral dosage form provided herein comprises an amount of 90 mg or 180 mg or 360 mg of a permeation enhancer. In an embodiment, the oral dosage form of the instant invention comprises an amount of 180 mg or 360 mg of a permeation enhancer.

When present in an oral dosage form, an amount of up to about 360 mg, an amount ranging from about 90 mg to about 360 mg, an amount ranging from about 180 to about 360 mg, or an amount of 90 mg, 180 mg or 360 mg of sodium caprate is used. In an embodiment, the oral dosage form provided herein comprises the permeation enhancer sodium caprate in the amount of 90 mg, 180 mg, or 360 mg. In an embodiment, 180 mg of sodium caprate is used in the oral dosage form. In an embodiment, 360 mg of sodium caprate is used in the oral dosage form.

In an embodiment, dry filled capsules or tablets may be used to administer the crystalline form of the compound of Formula I to a subject in need. In a pharmaceutical composition provided herein, a permeation enhancer may be included. In an embodiment, the amount of a permeation enhancer, such as sodium caprate, can range from about 1 wt % to about 75 wt %. As used herein, wt % refers to the weight percent of an ingredient relative to the total weight of the pharmaceutical composition. In another embodiment, the amount of a permeation enhancer in the pharmaceutical composition is from about 18 wt % to about 65 wt %. In a further embodiment, the amount of a permeation enhancer in the pharmaceutical composition is from about 22 wt % to about 36 wt %. For a tablet, the amount of permeation enhancer, such as sodium caprate, may range from about 22 wt % to about 65 wt %. Oral dosage forms may be manufactured by standard methods, including wet and dry granulation.

TABLE 27
Examples of formulations using Caprate 3

| | | | Formulation B |
|---|---|---|---|
| | | Formulation A* | Film Coating, |
| | | wt % | wt % |
| Caprate 3 | Active Pharmaceutical | 1 to 8 | 4 |
| | Ingredient (API) | | |
| Sodium Caprate | Permeation enhancer | 22.5 to 50 | 33 |
| Microcrystalline cellulose | Diluent | 20 to 36 | 29 |
| Lactose | Diluent | 20 to 36 | 29 |
| Croscarmellose sodium | Disintegrant | 0 to 3 | 3 |
| Silicon Dioxide | Glidant | 0 to 1 | 1 |
| Magnesium Stearate | Lubricant | 1 | 1 |
| Total core tablet or capsule fill | | 100 | 100 |
| Opadry ® TF Titanium free | Film coat | 0 to 8 | 6 |
| Carnauba wax | wax | 0 to 0.2 | 0.2 |

*Formulations A may be compressed into a tablet or filled into a capsule shell not limited to enteric, hard gelatin, or hypromellose

In an embodiment, provided herein is a pharmaceutical composition comprising a crystalline form of a compound of Formula I:

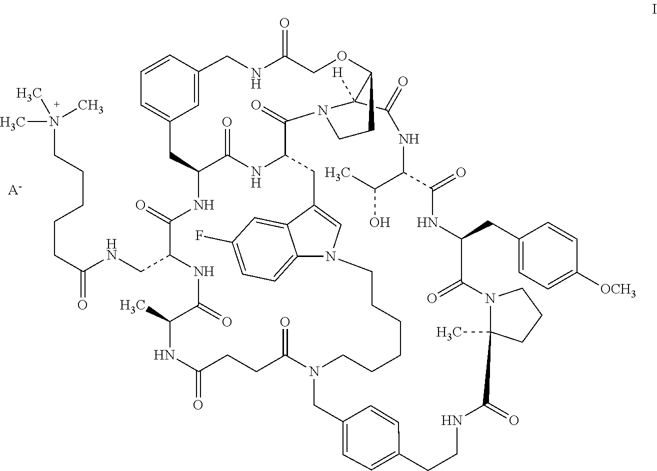

wherein A⁻ is a pharmaceutically acceptable anion, and a permeation enhancer. In a further embodiment, the permeation enhancer is sodium caprate. In another embodiment, the pharmaceutical composition further comprises a diluent. In a further embodiment, the composition comprises two or more diluents, wherein the two or more diluents comprise a combination of microcrystalline cellulose, macrogol (PEG 4000) and lactose. In a further embodiment, the composition comprises two or more diluents, wherein the two or more diluents comprise a combination of microcrystalline cellulose (Avicel PH 102), macrogol (PEG 4000) and lactose.

In an embodiment of the invention, the pharmaceutical composition comprises about 1% to about 8% of a crystalline form of a compound of Formula I by weight relative to the total weight of the pharmaceutical composition. In an embodiment, the pharmaceutical composition comprises about 1% to about 6% by weight of the crystalline form of a compound of Formula I relative to the total weight of the pharmaceutical composition. In another embodiment, the pharmaceutical composition comprises about 4% by weight of a crystalline form of a compound of Formula I relative to the total weight of the pharmaceutical composition.

In another embodiment of the invention, the pharmaceutical composition comprises about 22.5% to about 50% of the permeation enhancer by weight relative to the total weight of the pharmaceutical composition. In another embodiment, the pharmaceutical composition comprises about 33% of the permeation enhancer by weight relative to the total weight of the pharmaceutical composition.

In an embodiment of the invention, the pharmaceutical composition comprises at least one diluent. In an embodiment of the invention, the pharmaceutical composition comprises two diluents. In an embodiment, the diluent or combination of diluents comprise about 10% to about 70%, about 20% to about 60%, about 30% to about 50%, or about 40% to about 50% by weight of the diluents relative to the total weight of the pharmaceutical composition. In another embodiment, the pharmaceutical composition comprises about 15% to about 72% of diluents by weight relative to the total weight of the pharmaceutical composition. In an embodiment, the diluents comprise about 40% to about 72% by weight relative to the total weight of the pharmaceutical composition is present in the pharmaceutical composition. In an embodiment, the pharmaceutical composition comprises about 58% of diluents by weight relative to the total weight of the pharmaceutical composition.

In an embodiment of the invention, the pharmaceutical composition comprises a disintegrant. In an embodiment, the pharmaceutical composition comprises about 0% to about 3% of disintegrate by weight relative to the total weight of the pharmaceutical composition. In another embodiment, the pharmaceutical composition comprises about 3% of disintegrant by weight relative to the total weight of the pharmaceutical composition.

In an embodiment of the invention, the pharmaceutical composition comprises a glidant. In an embodiment, the pharmaceutical composition comprises about 0% to about 1% of glidant by weight relative to the total weight of the pharmaceutical composition. In another embodiment, the pharmaceutical composition comprises about 1% of glidant by weight relative to the total weight of the pharmaceutical composition.

In an embodiment of the invention, the pharmaceutical composition comprises a lubricant. In an embodiment, the pharmaceutical composition comprises about 1% to about 1.5% of lubricant by weight relative to the total weight of the pharmaceutical composition. In an embodiment, the pharmaceutical composition comprises about 1% of lubricant by weight relative to the total weight of the pharmaceutical composition.

In an embodiment of the invention, the pharmaceutical composition comprises a) about 1% to about 8% by weight relative to the total weight of the pharmaceutical composition of a crystalline form of a compound of Formula I; b) about 1% to about 80% by weight relative to the total weight of the pharmaceutical composition of a permeation enhancer; c) at least one diluent; d) optionally a glidant and/or a lubricant. In an embodiment, about 18% to about 74% by weight relative to the total weight of the pharmaceutical composition of a permeation enhancer is present in the pharmaceutical composition. In another embodiment of the invention, a pharmaceutical composition comprises a) about 1% to about 7% by weight relative to the total weight of the pharmaceutical composition of a crystalline form of a compound of Formula I; b) about 22% to about 67% by weight relative to the total weight of the pharmaceutical composition of a permeation enhancer selected from sodium caprate or Labrasol®; c) at least one diluent or solubilizing agent selected from PEG4000, microcrystalline cellulose, propylene glycol and lactose; d) optionally a glidant; and e) optionally a lubricant. In a further embodiment, the pharmaceutical composition further comprises about 22% to about 36%, by weight relative to the total weight of the pharmaceutical composition, of a permeation enhancer selected from sodium caprate or Labrasol®.

In an embodiment of the invention, provided herein is a pharmaceutical composition comprising a) about 1% to about 8% of a crystalline form of a compound of Formula I by weight relative to the total weight of the pharmaceutical composition; b) about 22.5% to about 80% of a permeation enhancer by weight relative to the total weight of the pharmaceutical composition, wherein the permeation enhancer is sodium caprate; c) two diluents selected from microcrystalline cellulose and lactose, wherein the combination of diluents comprise about 15% to about 72% by weight of diluent relative to the total weight of the pharmaceutical composition; d) optionally a disintegrant, wherein the pharmaceutical composition comprises about 0% to about 3% of the disintegrant by weight relative to the total weight of the pharmaceutical composition; e) optionally a glidant, wherein the pharmaceutical composition comprises about 0% to about 1% of glidant by weight relative to the total weight of the pharmaceutical composition, wherein the glidant is silicon dioxide; and f) about 1% to about 1.5% of a lubricant by weight relative to the total weight of the pharmaceutical composition, wherein the lubricant is magnesium stearate.

In an embodiment of the invention, provided herein is a pharmaceutical composition comprises a) about 1% to about 6% of a crystalline form of a compound of Formula I by weight relative to the total weight of the pharmaceutical composition; b) about 22.5% to about 50% of a permeation enhancer by weight relative to the total weight of the pharmaceutical composition, wherein the permeation enhancer is sodium caprate; c) two diluents selected from microcrystalline cellulose and lactose, wherein the combination of diluents comprise about 40% to about 72% by weight of diluent relative to the total weight of the pharmaceutical composition; d) about 3% of a disintegrant by weight relative to the total weight of the pharmaceutical composition; e) about 1% of a glidant by weight relative to the total weight of the pharmaceutical composition, where the glidant is silicon dioxide; and f) about 1% of a lubricant by weight relative to the total weight of the pharmaceutical composition, wherein the lubricant is magnesium stearate.

In an embodiment of the invention, provided herein is a pharmaceutical composition comprises a) about 4% of a crystalline form of a compound of Formula I by weight relative to the total weight of the pharmaceutical composition; b) about 33% of a permeation enhancer by weight relative to the total weight of the pharmaceutical composition, wherein the permeation enhancer is sodium caprate; c) two diluents selected from microcrystalline cellulose and lactose, wherein the combination of diluents comprise about 58% by weight of diluent relative to the total weight of the pharmaceutical composition; d) about 3% of a disintegrant by weight relative to the total weight of the pharmaceutical composition; e) about 1% of a glidant by weight relative to the total weight of the pharmaceutical composition, where the glidant is silicon dioxide; and f) about 1% of a lubricant by weight relative to the total weight of the pharmaceutical composition, wherein the lubricant is magnesium stearate.

In an embodiment of the invention, a pharmaceutical composition comprises a) about 2% to about 6% by weight relative to the total weight of the pharmaceutical composition of a crystalline form of a compound of Formula I; b) about 18% to about 74% by weight relative to the total weight of the pharmaceutical composition of a permeation enhancer, where the permeation enhancer is sodium caprate; c) at least one diluent selected from PEG4000, microcrystalline cellulose or lactose; d) about 0% to about 3% by weight relative to the total weight of the pharmaceutical composition of a glidant, where the glidant is silicon dioxide; e) about 0% to about 2% by weight relative to the total weight of the pharmaceutical composition of a lubricant where the lubricant is magnesium stearate and f) optionally at least one disintegrant.

In an embodiment of the above, the diluent comprises about 10% to about 70%, about 20% to about 60%, about 30% to about 50%, or about 40% to about 50% by weight relative to the total weight of the pharmaceutical composition.

In another embodiment of the invention, provided herein is a pharmaceutical composition comprising a) about 4% by weight relative to the total weight of the pharmaceutical composition of a crystalline form of a compound of Formula I; b) about 33% by weight relative to the total weight of the pharmaceutical composition of a permeation enhancer, where the permeation enhancer is sodium caprate; c) about 58% by weight relative to the total weight of the pharmaceutical composition of one or more diluents selected from, microcrystalline cellulose or lactose; d) about 1% by weight relative to the total weight of the pharmaceutical composition of a glidant, where the glidant is silicon dioxide; e) about 1% by weight relative to the total weight of the pharmaceutical composition of a lubricant where the lubricant is magnesium stearate and f) about 3% by weight relative to the total weight of the pharmaceutical composition of at least one disintegrant.

In an embodiment, provided herein is a pharmaceutical composition comprising Caprate 3, a pharmaceutically acceptable anion, and a permeation enhancer. In a further embodiment, the permeation enhancer is sodium caprate. In another embodiment, the pharmaceutical composition further comprises a diluent. In a further embodiment, the composition comprises two or more diluents, wherein the two or more diluents comprise a combination of microcrystalline cellulose, macrogol (PEG 4000) and lactose.

In an embodiment of the invention, the pharmaceutical composition comprises a) about 1% to about 8% by weight relative to the total weight of the pharmaceutical composition of Caprate 3; b) about 1% to about 75% by weight relative to the total weight of the pharmaceutical composition of a permeation enhancer; c) at least one diluent; d) optionally a glidant and/or a lubricant. In an embodiment, about 18% to about 74% by weight relative to the total weight of the pharmaceutical composition of a permeation enhancer is present in the pharmaceutical composition. In another embodiment of the invention, a pharmaceutical composition comprises a) about 1% to about 8% by weight relative to the total weight of the pharmaceutical composition Caprate 3; b) about 22% to about 67% by weight relative to the total weight of the pharmaceutical composition of a permeation enhancer selected from sodium caprate or Labrasol®; c) at least one diluent or solubilizing agent selected from PEG4000, microcrystalline cellulose, propylene glycol and lactose; d) optionally a glidant; and e) optionally a lubricant.

In an embodiment of the above, the diluent or solubilizing agent comprises about 10% to about 70%, about 20% to about 60%, about 30% to about 60%, or about 40% to about 60% by weight relative to the total weight of the pharmaceutical composition.

In an embodiment of the invention, a pharmaceutical composition comprises a) about 2% to about 6% by weight relative to the total weight of the pharmaceutical composition of Caprate 3; b) about 18% to about 74% by weight relative to the total weight of the pharmaceutical composition of a permeation enhancer, where the permeation enhancer is sodium caprate; c) at least one diluent selected from PEG4000, microcrystalline cellulose or lactose; d) 0% to about 3% by weight relative to the total weight of the pharmaceutical composition of a glidant, where the glidant is silicon dioxide; e) 0% to about 2% by weight relative to the total weight of the pharmaceutical composition of a lubricant where the lubricant is magnesium stearate and f) optionally at least one disintegrant.

In an embodiment of the above, the diluent or solubilizing agent comprises about 10% to about 70%, about 20% to about 60%, about 30% to about 50%, or about 40% to about 50% by weight relative to the total weight of the pharmaceutical composition.

In another embodiment of the invention, provided herein is a pharmaceutical composition comprising a) about 4% by weight relative to the total weight of the pharmaceutical composition Caprate 3; b) about 33% by weight relative to the total weight of the pharmaceutical composition of a permeation enhancer, where the permeation enhancer is sodium caprate; c) about 58% by weight relative to the total weight of the pharmaceutical composition of one or more diluents selected from PEG4000, microcrystalline cellulose or lactose; d) about 1% by weight relative to the total weight of the pharmaceutical composition of a glidant, where the glidant is silicon dioxide; e) about 1% by weight relative to the total weight of the pharmaceutical composition of a lubricant where the lubricant is magnesium stearate and f) about 3% by weight relative to the total weight of the pharmaceutical composition of at least one disintegrant.

The disclosure is further illustrated by the following examples and synthesis, which are not to be construed as limiting this disclosure in scope or spirit. Those of skill in the art will readily recognize a variety of non-critical parameters which can be changed or modified to yield essentially the same or similar results.

### EXAMPLES

### Examples for Preparing Crystallized Forms of Compounds of Formula I

### Synthesis of a Compound of Formula I

A compound of Formula I and methods for making the same are illustrated in PCT International Patent Application Publication No. WO 2019/246349, which is incorporated herein by reference in its entirety. As demonstrated in Example 25 of International Patent Application Publication No. WO 2019/246349, Compound A has a Kᵢ Plus of 0.01127 nm and a Kᵢ Ultra of 0.00463 nm as demonstrated in an PCSK9 Alexa FRET Ultra assay.

### Example 1A: Preparation of Lyophilized Acetate 1 (Amorphous Acetate Salt)

Macroporous anion exchange resin AG MP-1M (6 g, 100-200 mesh, chloride form) was packed in a 60 mL funnel. The packed resin was washed with 9 mL of the mixture of acetonitrile (MeCN)/water (1:1 ratio), 5 times. The resin was washed with 200 mL of 1M sodium hydroxide (NaOH) and then with 50 mL of IM acetic acid (AcOH) in water. The resin was transferred to a 100 mL round bottom flask containing a solution of Compound A (0.3 g) in 6 mL of a 1:1 mixture of acetonitrile and water. An additional 18 mL of MeCN/water (1:1) was added. The mixture was aged at room temperature for 30 minutes and the resulting mixture was transferred into a 60 mL funnel. The filtrate was collected in a 20 mL vial, and the resin was washed with 10 mL MeCN/water (1/1), three times, and the filtrate was collected in 20 ml vials. The fractions containing Acetate I were combined and concentrated, to remove MeCN, and then the desired amorphous Acetate 1 (0.304 g) was isolated via lyophilization of the solution.

### Example 1B: Alternative Preparation of Acetate 1

Macroporous anion exchange resin AG 1-X2 (8.1 g, 100-200 mesh, acetate form) was packed in a 100 mL filter funnel. The resin was washed with water (UPLC LC-MS grade, 5×12.5 mL, the first three wash fractions were not clear, continued to wash the resin by slurrying and applying vacuum till the eluate was clear). The resin was transferred to Redi Sep Rf (Teledyne ISCO) empty solid load cartridge using 10 mL of water with gravity elution. Compound A (0.3 g, 0.189 mmol) was dissolved in 3 mL of water. The solution of Compound A was loaded to the cartridge. The resulting compound, Acetate I was eluted with water (25 mL). Acetate 1 (0.29 g) was isolated via lyophilization of the solution.

### Example 2: Preparation of Lyophilized Caprate 1 (Amorphous Caprate Salt)

Macroporous anion exchange resin AG MP-1M (6 g, 100-200 mesh, chloride form) was packed in a 60 mL funnel. The packed resin was washed with 9 mL of a mixture of acetonitrile and water (1:1), five times. The resin was washed with 200 mL of IM NaOH and then with 10 mL of water, two times. The resin was transferred to a glass column and washed with 10 mL of water, three times. The resin was then washed with 10 mL of ethanol (EtOH), two times, and then with 9 mL of IM capric acid solution in EtOH, two times, followed by 9 mL of EtOH, three times. Compound A (0.3 g) was dissolved in 6 mL MeCN/water (1:1) and loaded into the resin-packed column. The filtrate was collected in a 20 mL vial. The column was washed with 15 mL of MeCN and water solution (1:1), three times, and the filtrate was collected in 20 ml vials. The fractions containing Caprate 1 were combined and concentrated, to remove MeCN, and then the desired amorphous Caprate 1 (0.29 g) was isolated via lyophilization.

### Example 3: Preparation of Acetate 2

Acetate 1 (25.5 mg) was added to a vial, and 2-Me-THF (250 uL) was charged. The slurry was aged at room temperature. n-BuOH (150 uL) was charged, and the mixture was aged until it became homogenous. The homogeneous solution was aged at room temperature for 4 days, which provided a white slurry. The microscope image of the slurry revealed that the slurry had needle-shaped crystals (Acetate 2).

### Example 4: Preparation of Acetate 3

To a round bottom flask, Acetate 1 (37 g) was dissolved in 3 volumes of 1-propanol (111 ml). The solution was aged for 20 minutes at 25° C. 2-Me-THF (46.7 mL) was charged. Acetate 2 seeds were charged as a slurry and the mixture was aged for 40 minutes. The remaining seed slurry was charged. 2-Me-THF (174 ml) was charged over 10 hours at 25° C. The mixture was aged for 4 hours after the 2-Me-THE addition to provide Acetate 3.

### Example 5: Preparation of Acetate 4

The mixture from Example 4 (Acetate 3) was vacuum filtered, and the wet cake was washed with a n-propanol-2-Me-THE mixture (1:4.3, w/w), followed by a second wash of 2-Me-THF. The solids were dried with nitrogen (N₂) at ambient temperature for 4 days and provided Acetate 4.

### Example 6: Alternative Preparation of Acetate 1

Acetate 4 (1.755 g) was added to a vial. Wet MeTHF (2 wt % water in 2-methyltetrahydrofuran (2-MeTHF, 19.99 g) was charged and the resulting slurry was aged at room temperature. The slurry was filtered, and the wet cake was dried under vacuum with air sweep over 1 hour. A white solid of Acetate 1 (1.75 g) was obtained.

### Example 7: Preparation of Acetate 5

Acetate 1 (4.787 g) was dissolved in 14.4 mL nPrOH. 2-MeTHF (6.2 mL) was charged over 5 minutes while stirring. Karl Fischer (KF) of the resulting solution was 7628 ppm. An additional 2 mL of 2-MeTHF was charged. The seeds of Acetate 2 were charged as a slurry. The resulting slurry was aged at room temperature for 15 minutes. 2-MeTHF (20.7 mL) was slowly charged over 5 hours at room temperature. The slurry was aged for an additional 22 hours after the addition was completed. The slurry was filtered, and the wet cake was washed with 5 mL 2-MeTHF, two times and provided Acetate 5.

### Example 8: Preparation of Acetate 6

The wet cake from Example 7 (Acetate 5) was dried under vacuum with N₂ sweep. A white solid was obtained as Acetate 6.

### Example 9: Preparation of Caprate 4

To a glass vessel, Caprate 1 (40 g) was dissolved in 3 volumes of 1-propanol (120 ml). The solution was aged for 20 min at 20° C. MTBE was added to solution (14.4 ml). The mixture was heated to 28-28.5° C. to achieve dissolution. The solution was cooled to 25° C. and the resulting Caprate 4 seeds were charged as a slurry. The mixture was aged for 20 minutes, followed by MTBE solvent addition (225.6 ml) over 10 hours. The suspension was aged at 25° C. for 8.5 hours to afford Caprate 4.

### Example 10: Preparation of Caprate 5

The suspension of Example 10 (Caprate 4) was filtered and washed with MTBE-20% 1-propanol. The solids were dried under blanket of N₂ over an extended period of time (˜118 hr) to remove 1-propanol and MTBE and afford Caprate 5.

### Example 11: Preparation of Caprate 2

To a vial, Caprate 5 (1.838 g) was added, followed by a ternary solvent mixture containing methyl tert-butyl ether (MTBE) with 38.3 weight % n-propanol and 0.99 weight % water. Mixture aged for 3 hours to provide Caprate 2.

### Example 12A: Preparation of Caprate 3

The mixture of Example 11 (Caprate 2) was filtered by centrifugation with the use of a centrifugal filter. The cake naturally air-dried under ambient conditions for 2 hours to provide Caprate 3.

### Example 12B: Alternative Preparation of Caprate 3

Compound A (chloride salt, 700 g) was dissolved in a 4/1 mixture of acetonitrile-water (8.4 L) at 20° C. and the solution was warmed to 35° C. The solution was mixed with 3.0 M aqueous KHCO₃ (7.0 L; the solution of KHCO₃ was prepared and kept at 35° C. to prevent precipitation), stirred for 10 min, and the layers were separated while maintaining the temperature at 35° C. The organic phase was combined with a 4/1 mixture of acetonitrile-water (0.7 L) and 3.0 M aqueous KHCO₃ (7.0 L), stirred for 10 min, and the layers were separated while maintaining the temperature at 35° C. The organic phase was again combined with a 4/1 mixture of acetonitrile-water (0.7 L) and 3.0 M aqueous KHCO₃ (7.0 L), stirred for 10 min, and the layers were separated while maintaining the temperature at 35° C. The resulting organic phase containing Compound B (bicarbonate salt) was cooled at 20° C. and 1-propanol (7.0 L) was added. The heterogenous solution was cooled at 4° C. and aged overnight. The mixture was filtered, and the filter was rinsed with 1-propanol (1.4 L). To the combined filtrate was added decanoic acid (93 g) and the mixture was stirred for 15 min at rt to achieve dissolution. Acetonitrile solvent was replaced with 1-propanol by continuous distillation under vacuum. 1-Propanol was added to the concentrated residue until reaching a total volume of 1-propanol of 2.0 L. Water (36 mL) was added to reach 2.3 wt % of water relative to 1-propanol. The mixture was stirred at room temperature and MTBE (0.984 L) was added (Solution #1). A separate flask was charged MTBE (2.95 L) (Solution #2).

In a separate vessel, the seed bed was prepared by addition of 25 g of Caprate 3 seeds following by 0.6 L of a solution of 2/1 MTBE/1-PrOH containing 0.5 wt % water. The resulting slurry was aged at 22° C. for 1 h to obtain Caprate 2 seed slurry.

Solutions #1 and #2 were simultaneous added to a stirred Caprate 2 seed slurry over the course of 6 h while maintaining temperature at 22° C. The resulting slurry was aged overnight at 22° C. to afford Caprate 2 slurry.

Caprate 2 slurry was filtered under nitrogen atmosphere. The wet cake was washed with 1.4 L of a solution of 8/2 (m: m) MTBE/1-PrOH containing 0.5 wt % water. The cake was dried under a nitrogen flow in order to remove MTBE and part of the 1-PrOH solvent.

Humid drying with humidified nitrogen (50% RH) was applied to remove residual 1-PrOH affording Caprate 3 (684 g).

### Example 12C: Alternative Preparation of Caprate 3

To a mixture of MTBE (6.75 mL) and n-propanol (3.37 mL) was added water (54 μl) to ensure that the water content in the solution is at least 0.7 wt %. Caprate 3 seed crystals (120 mg) were added, and the slurry was stirred for 1 h at 22° C. to provide the seed slurry of Caprate 2.

In a separate mixture, Caprate 3 (5 g, containing 6.7 wt % water) was dissolved in n-propanol (13.8 mL). MTBE (6.9 mL) was added to the solution to dilute it. Capric acid (120 mg) was added to the solution.

Both solutions were added simultaneously into the stirred seed slurry over 20 h at 22° C. A solution of MTBE (6.13 mL), n-propanol (3.07 mL), and water (49 μL) was used to rinse the addition lines into the slurry. The resulting slurry of Caprate 2 was filtered. The filter cake was washed with a solution of MTBE (8.30 g), n-propanol (2.07 g), and water (52 μL). The filter cake was dried by passing nitrogen through the filter funnel to surface-dry the batch. The batch was then dried under humidified nitrogen at 250 mm Hg and relative humidity of 50% to provide Caprate 3 (4.01 g).

### Example 13: Alternative Preparation of Caprate 4

To a vial, Caprate 3 (101.9 mg) was added, followed by 1-propanol-MTBE (1:1.1) solvent mixture (1 ml). The mixture was agitated at 5° C. and aged overnight to afford Caprate 4.

### Example 14: Preparation of Caprate 6

To a vial, Caprate 1 (0.5 g) was added. Next, n-propanol (1.35 mL) was charged to achieve dissolution. Ethyl acetate (7.2 mL) was charged, followed by cooling to sub-ambient temperature. An additional 2.7 ml of ethyl acetate was charged to the solution. The mixture was aged overnight to obtain Caprate 6.

### Example 15: Preparation of Caprate 7

The suspension of Example 14 (Caprate 6) was vacuum filtered, and the wet cake was washed with 1-propanol-ethyl acetate mixture (1:10). The cake was dried overnight in a vacuum oven at 30° C. with a dry nitrogen sweep to afford Caprate 7.

### Example 16: Preparation of Caprate 8

The cake of Example 9 (Caprate 4) was vacuum filtered, and the wet cake was washed with a n-propanol-MTBE mixture (1:4, w/w), followed by a second wash of MTBE (120 ml). The solids were dried with N₂ at ambient temperature to afford Caprate 8.

### Example 17: Alternative Preparation of Caprate 1

Caprate 8 was stressed at 97% RH for at least 3 days followed by drying at 40° C. under reduced pressure with nitrogen sweep over 1 hour providing Caprate 1.

### Example 18: Preparation of Caprate 9

A mixture of Caprate 3, Caprate 5, and Caprate 7 (0.01:1:1) was suspended in a 1-propanol-MTBE (1:12, v/v) solvent mixture at room temperature for at least a week to obtain Caprate 9.

### Example 19: Alternative Preparation of Caprate 3

The wet cake of Example 18 (Caprate 9) was vacuum filtered, and dried overnight in a vacuum oven with a dry nitrogen sweep to afford Caprate 3.

### Example 20: Preparation of Caprate 10

Caprate 3 was exposed to 5% RH for at least 3 hr to obtain Caprate 10.

### Example 21: Preparation of Caprate 11

Caprate 7 was exposed to 5% RH for at least 3 hr to obtain Caprate 11.

### Example 22: Preparation of Caprate 12

A mixture of Caprate 3, Caprate 5, and Caprate 7 (0.01:1:1) was suspended in 1-propanol-MTBE (1:1, v/v) mixture at 5° C. for one week to obtain Caprate 12.

### Example 23: Preparation of Caprate 13

Caprate 3 was exposed to 1-propanol solvent vapor for at least three days to obtain Caprate 13.

### Example 24: Preparation of Caprate 14

Caprate 3 was exposed to 1-propanol-MTBE solvent vapor for at least three days to obtain Caprate 14.

### Example 25: Preparation of Compound B (Bicarbonate Salt)

Macroporous anion exchange resin AG MP-1M (chloride form, 100-200 mesh, 160 g) was loaded in a 500 mL filter funnel. The resin was washed with water (UPLC LC-MS grade, 5×264 mL, the first three wash fractions were not clear, continued to wash the resin by slurrying and applying vacuum till the eluate was clear). The resin in the filter funnel was converted to HCO₃-anion form by being eluted with 2.5 bed volumes of 5 wt % NaHCO₃ in water (2.5×265 mL) with some slurrying. The resin was transferred into a Redi Sep Rf (Teledyne ISCO, Diameter: 2.42 inches) empty solid load cartridge using 100 mL 5 wt % NaHCO₃ solution in water with gravity elution. The cartridge was eluted with 7.5 bed volumes of 5 wt % NaHCO₃ in water (7.5×265 mL) with gravity elution. Excess NaHCO₃ was washed out with 2×265 mL water with gravity elution. Compound A (10 g, 6.14 mmol) was dissolved in 100 ml of water. The solution of Compound A was loaded to the cartridge and was rinsed with 10 ml of water. The resulting compound, Compound B (the bicarbonate salt) was eluted with water (260 mL).

### Example 26: Preparation of D-Lactate 1

A solution of D-lactic acid (280 mg, 3.07 mmol) in water was added to a solution of Compound B (3.07 mmol) in water. The solution was aged at 0° C. for 30 min. Then the solution was frozen in a dry ice-acetone bath and lyophilized overnight to provide 5.0 g of lyophilized D-lactate salt of a compound of Formula I. The lyophilized D-lactate salt of a compound of Formula I (5.0 g) was dissolved in an ethanol/2-Me-THF mixture (1:1, 20 mL). The solution was transferred to a 250 mL 3-neck round bottom flask with overhead stirrer and N₂ inlet. The transfer was completed by rinsing the flask with an ethanol: 2-Me-THF (1:1, 10 mL) and adding the rinse to the 250 mL 3-neck round-bottom flask. 2-Me-THF (10 mL) was added dropwise via syringe. The addition was stopped and seeded using a slurry of seed crystals. After 1 hour, a proper slurry had formed. An ethanol/2-Me-THF mixture (1:3) was added, followed by 2-Me-THF was added (20 mL) via syringe pump over 2.5 hours. The slurry was aged overnight to provide D-Lactate 1.

### Example 27: Preparation of D-Lactate 2

A slurry of D-Lactate 1 (˜5 g) was filtered using some filtrate to complete the transfer. The cake was washed with 2-Me-THF: EtOH (3:1, 7 mL), followed by 2-Me-THF (10 mL), followed by heptane (20 mL). The solid was dried with vacuum under a blanket of nitrogen to provide D-Lactate 2 (3.9 g).

### Example 28: Preparation of Succinate 1

A solution of succinic acid (362.9 mg, 3.07 mmol) in water was added to a solution of Compound B (167 g; 3.07 mmol) in water and aged at room temperature for 1 h. Then the solution was frozen in a dry ice-acetone bath and lyophilized overnight to provide lyophilized succinate salt of a compound of Formula I (5.05 g). A mixture of succinate salt of a compound of Formula I (3.50 g, 2.097 mmol) and EtOH (17.5 ml) was evaporated under nitrogen stream at 15-25° C. to a gum. Under nitrogen, EtOH (17.5 mL) was added, and the mixture was evaporated under nitrogen stream at 45-55° C. to a gum. The residue was dissolved in EtOH (17.5 mL) at 75° C. to give a homogeneous solution. The mixture was cooled to 25° C. and seed crystals (1 mg) were added after which solid slowly crystallized out. The mixture was stirred for 16 hours and then cooled to 1-3° C. for 2 hours to provide Succinate 1.

### Example 29: Preparation of Succinate 2

A suspension of Succinate 1 (˜3.5 g) was filtered on a 30cc polypropylene filter funnel using EtOH (10 mL) at 0-5° C. to completely transfer the slurry to the filter funnel. The filter cake was dried under nitrogen stream for 24 hours to give Succinate 2 (2.8 g, 1.678 mmol, 80% yield) as a white crystalline solid.

### Example 30: Preparation of L-Tartrate 1

A solution of L-(+)-tartaric acid (460.9 mg, 3.07 mmol) in water was added to a solution of Compound B (167 g; 3.07 mmol) in water and aged at room temperature for 1 hour. Then the solution was frozen in a dry ice-acetone bath and lyophilized overnight to provide lyophilized L-tartrate salt of a compound of Formula I (5.15 g). Lyophilized L-tartrate salt of a compound of Formula I (5.0 g) and n-propanol (50 mL) was charged into a 100 mL EasyMax vessel with a nitrogen blanket to control humidity. The mixture was agitated and heated to 55° C. to dissolve all solids. The solution was cooled to 50° C. and seed crystals were charged. The slurry was slowly cooled to 45° C. and then several heating and cooling cycles were performed to crystallize the product. In the final cycle, the slurry was heated to 40° C. and cooled to 20° C. over 4 h to provide L-Tartrate 1.

### Example 31: Preparation of L-Tartrate 2

L-Tartrate 1 (˜5 g) was filtered, and the cake was washed with n-propanol. The cake was dried in the oven at 40° C. with a nitrogen sweep overnight to provide L-Tartrate 2 (2.57 g).

### Example 32: Preparation of Sulfate 1

A solution of sulfuric acid in water (1M, 1.5 mL, 1.5 mmol) was added to a solution of Compound B (167 g; 3.07 mmol) in water and was aged at room temperature for 1 hour. Then the solution was frozen in a dry ice-acetone bath and lyophilized overnight to provide lyophilized sulfate salt of a compound of Formula I (4.88 g). A 250 mL 3-neck round bottom flask equipped with an overhead stirrer was charged with lyophilized sulfate salt of a compound of Formula I (2.5 g). 1-propanol (40 mL) was added to the round bottom flask and stirred vigorously to dissolve with some solids sticking to the wall of the flask. Crystals were formed prior to all solids dissolving. The mixture continued to be vigorously stirred and 10 mL heptane over 1 hour via syringe pump and aged for 6 hours providing Sulfate 1.

### Example 33: Preparation of Sulfate 2

A suspension of Sulfate 1 (˜4.9 g) was filtered, and the cake was washed with 10 mL of a mixture of 1-propanol and 20% heptane. Then the cake was washed with 20 mL heptane followed by additional 10 mL heptane. The cake was dried with vacuum under a blanket of nitrogen overnight to provide Sulfate 2 (2.21 g).

### Example 34: Description of Powder X-Ray Diffraction Studies

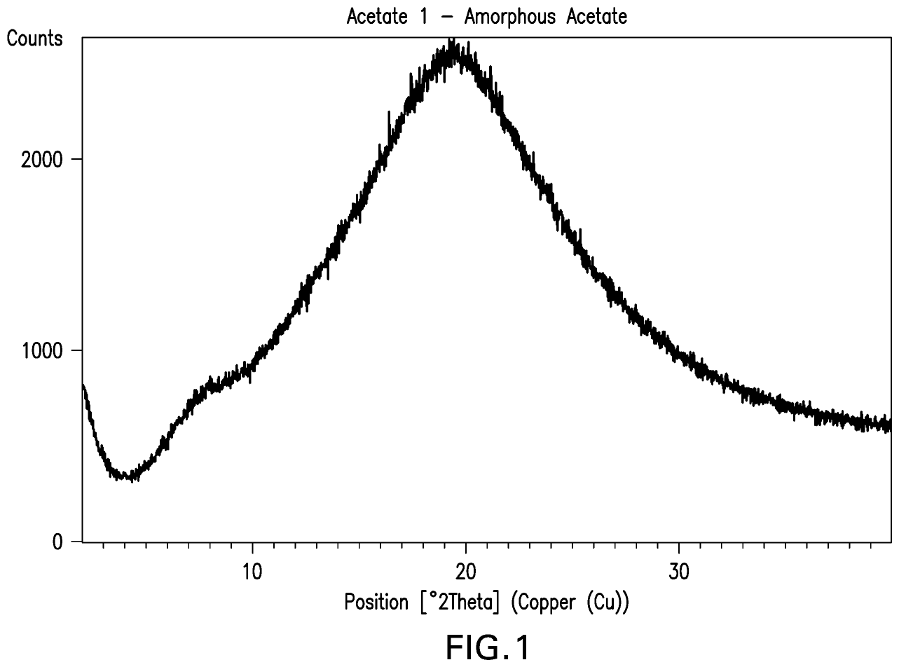

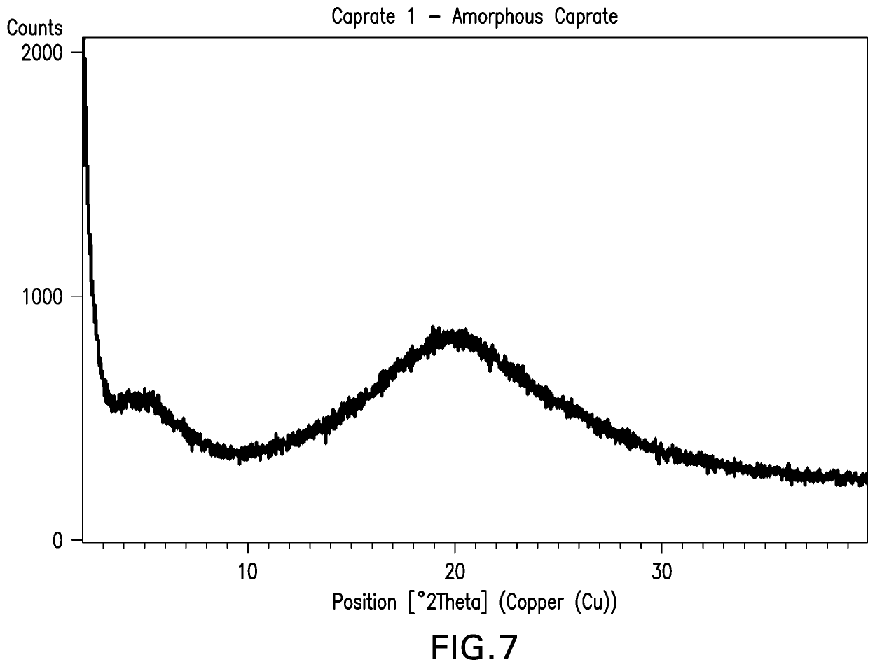

X-ray powder diffraction studies are widely used to characterize molecular structures, crystallinity, and polymorphism. The X-ray powder diffraction patterns disclosed herein were generated on a Philips Analytical X′Pert PRO X-ray Diffraction System with PW3040/60 console. A PW3373/00 ceramic Cu LEF X-ray tube K-Alpha radiation was used as the source. Samples of Examples 1-33 were characterized by XRPD. XRPD analysis shows that Acetate 1 (FIG. 1) and Caprate 1 (FIG. 7) are amorphous and that Acetate 2-6 (FIG. 2-FIG. 6), Caprate 2-14 (FIG. 8-FIG. 20), D-Lactate 1-2 (FIG. 21-FIG. 22), Succinate 1-2 (FIG. 23-FIG. 24), L-Tartrate 1-2 (FIG. 25-FIG. 26), and Sulfate 1-2 (FIG. 27-FIG. 28) are crystalline.

### Example 35: Chemical Stability of Crystalline Salts Over Compound A (the Amorphous Chloride Salt)

The crystalline forms disclosed herein provide improved chemical purification advantages. In particular, these crystalline forms avoid the use of SFC chromatography and lyophilization necessary for the purification of Compound A (the chloride salt). This improved strategy reduces the cost of goods and results in a process simplification as there is a decrease in the number of units of operations involved which is critical for commercial viability.

The acetate and caprate salts of Formula I, including crystalline forms Caprate 3 and Caprate 7, demonstrated a satisfactory purity of >99% at 40° C. and 75% RH for a range of 3 months (FIG. 29A) in contrast to the purity of the amorphous chloride. Compound A. The graph of FIG. 29A demonstrates that the acetate and caprate salts offer enhanced chemical stability over the chloride salt. In addition, the chloride salt requires −20° C. storage to minimize chemical degradation while the acetate and caprate salts are less prone to degradation at higher temperatures. The graph of FIG. 29B demonstrates that the acetate and caprate crystalline salts offer enhanced chemical stability over the amorphous forms of caprate and acetate salts at accelerated stability conditions, which is especially evident at the three-month time point. The stability of crystalline forms of the compounds of Formula I were characterized by XRPD under relative humidity conditions that simulate potential storage conditions.

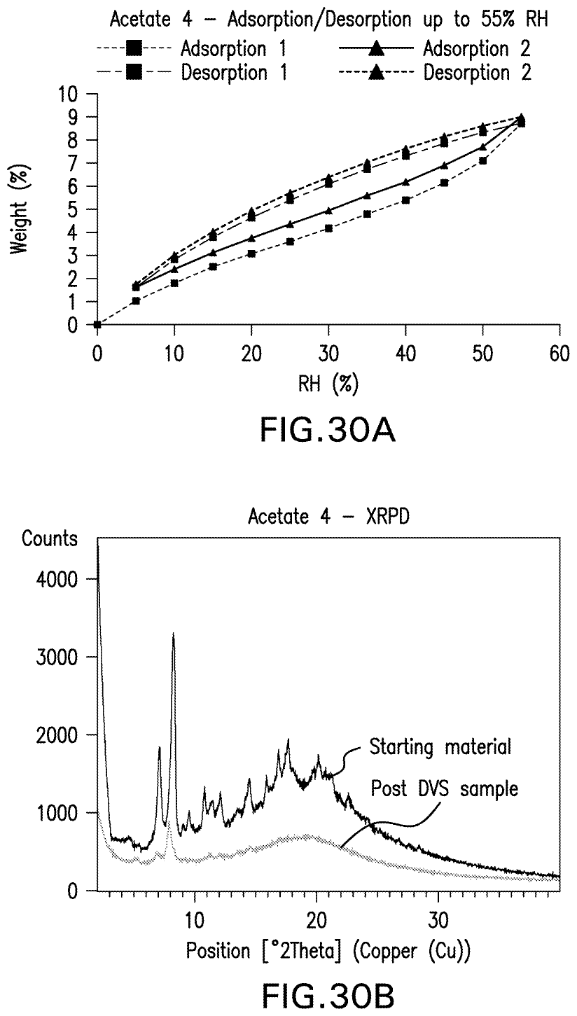

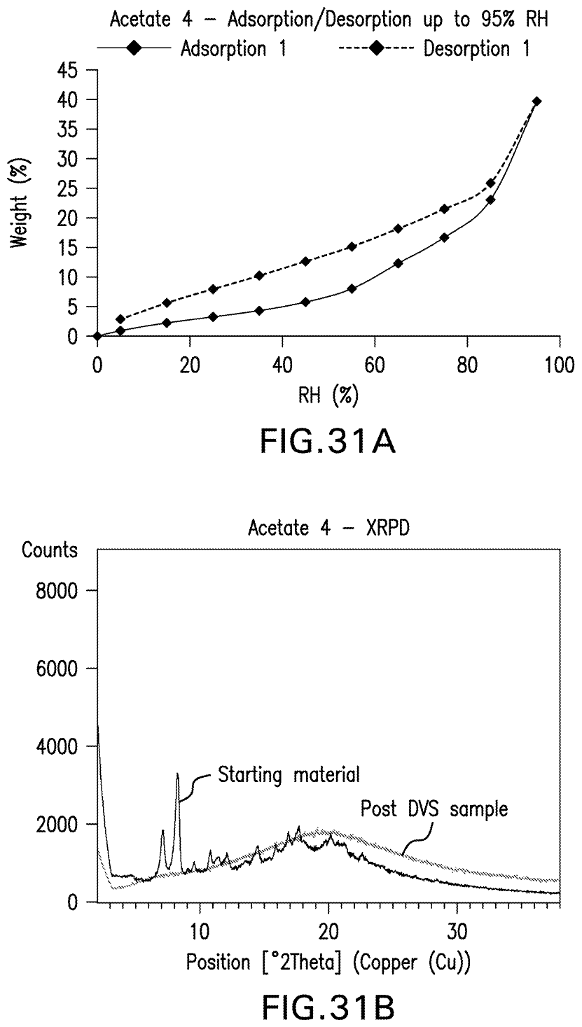

Adsorption/desorption cycles show that Acetate 4 is hygroscopic with approximately a 9% increase in weight at 55% RH (see FIG. 30A). XRPD analysis shows that Acetate 4 retains minimal crystallinity upon two adsorption/desorption cycles of 5-55% RH (see FIG. 30B). Adsorption/desorption cycles show that Acetate 4 is very hygroscopic with approximately a 40% increase in weight at 95% RH (see FIG. 31A). Hysteresis is observed during the desorption step. XRPD analysis shows that Acetate 4 loses crystallinity after the 5-95-5% RH adsorption/desorption cycle (see FIG. 31B).

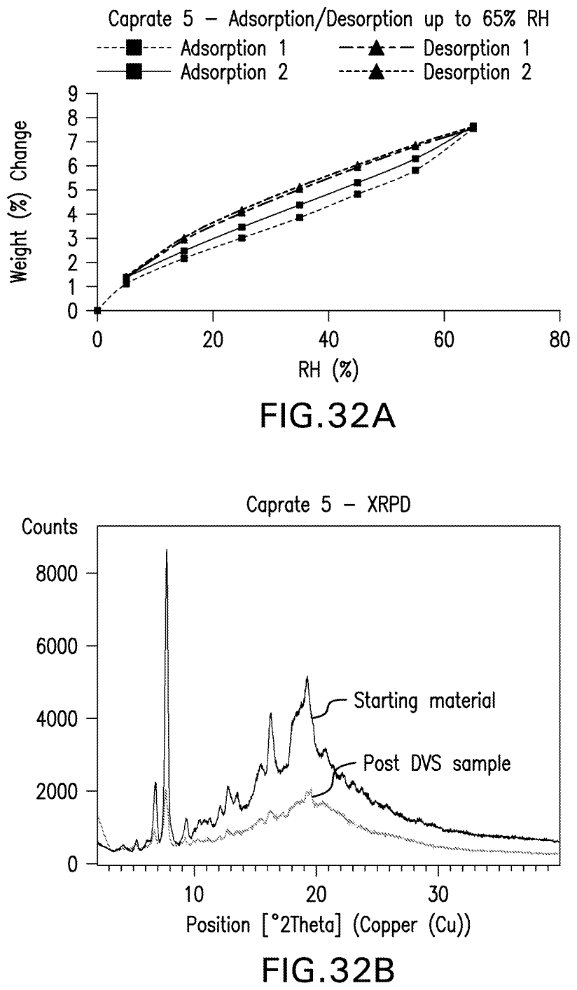

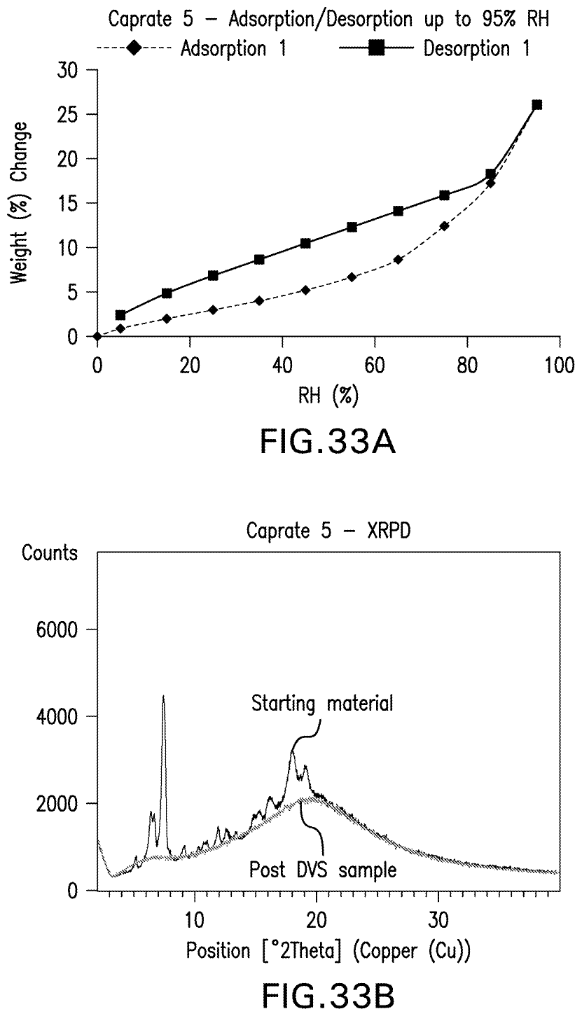

Adsorption/desorption cycles show that Caprate 5 is hygroscopic with approximately a 7.5% increase in weight at 65% RH (see FIG. 32A). Slight hysteresis is observed during the desorption steps of Cycle 1 and Cycle 2. XRPD analysis shows that Caprate 5 retains some crystallinity upon two adsorption/desorption cycles of 5-65% RH (see FIG. 32B). Adsorption/desorption cycles show that Caprate 5 is very hygroscopic with approximately a 26% increase in weight at 95% RH (see FIG. 33A). XRPD analysis shows that Caprate 5 loses crystallinity after the 5-95-5% RH adsorption/desorption cycle (see FIG. 33B).

Adsorption/desorption cycles show that Caprate 3 is hygroscopic with approximately a 4.9% increase in weight at 85% RH (see FIG. 34A). This is in contrast to the larger increase in weight observed for Acetate 4 (9%; see FIG. 30A) and Caprate 5 (7.5%; see FIG. 32A), at 55% RH and 65% RH, respectively. XRPD analysis shows that Caprate 3 retains crystallinity upon adsorption/desorption cycles of 5-85% RH (see FIG. 34B). This is in contrast to the decrease in crystallinity observed for Acetate 4 (FIG. 30B) and Caprate 5 (see FIG. 32B) upon two adsorption/desorption cycles of 5-55% RH and 5-65% RH, respectively.

Caprate 3 was dried at 40° C. under N₂ for 3 hours to remove residual. Adsorption/desorption cycles show that the water-free Caprate 3 is hygroscopic with approximately a 9.4% increase in weight at 85% RH (see FIG. 35A). This adsorption/desorption cycle shows that the Caprate 3 readily absorbs H₂O by approximately 25-35% RH and does not lose H₂O until 15% RH. XRPD analysis shows that the water-free Caprate 3 retains crystallinity upon adsorption/desorption cycles of 5-85% RH (see FIG. 35B).

The behavior of Caprate 3 indicates a high stability of this crystalline form with respect to relative humidity, which is important for further development and especially for withstanding variable storage conditions. This is in contrast to the decrease in crystallinity observed in Acetate 4 and Caprate 5 with increases in relative humidity.

### Example 36: Solid State NMR Studies

Caprate 3, Caprate 5, and Caprate 8 batches were characterized based on the respective solid-state carbon-13 nuclear magnetic resonance (NMR) spectra. All carbon-13 spectra were recorded on a Bruker AV400 NMR spectrometer operating at a carrier frequency of 400.14 MHz, using a Bruker 4 mm H/F/X BB triple resonance CPMAS probe. The spectra were collected utilizing proton/carbon-13 variable-amplitude cross-polarization (VACP) at 80 kHz, with a contact time of 3 minutes. Other experimental parameters used for data acquisition were a proton 90-degree pulse of 100 kHz, SPINAL64 decoupling at 100 kHz, a pulse delay of 1.5 s, and signal averaging for 50000 scans. The magic-angle spinning (MAS) rate was set to 13 kHz. A Lorentzian line broadening of 30 Hz was applied to the spectra before Fourier Transformation. Chemical shifts are reported on the TMS scale using the carbonyl carbon of glycine (176.70 5 ppm.) as a secondary reference.

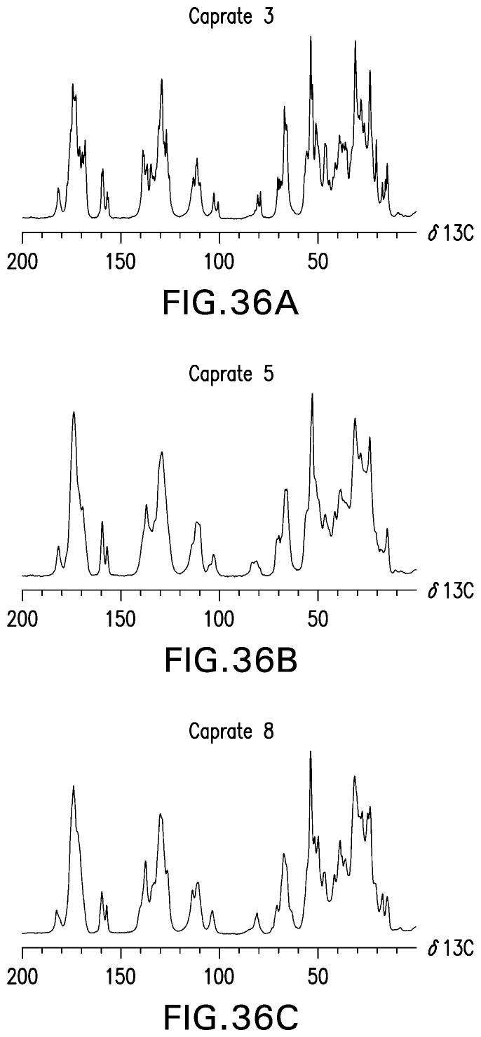

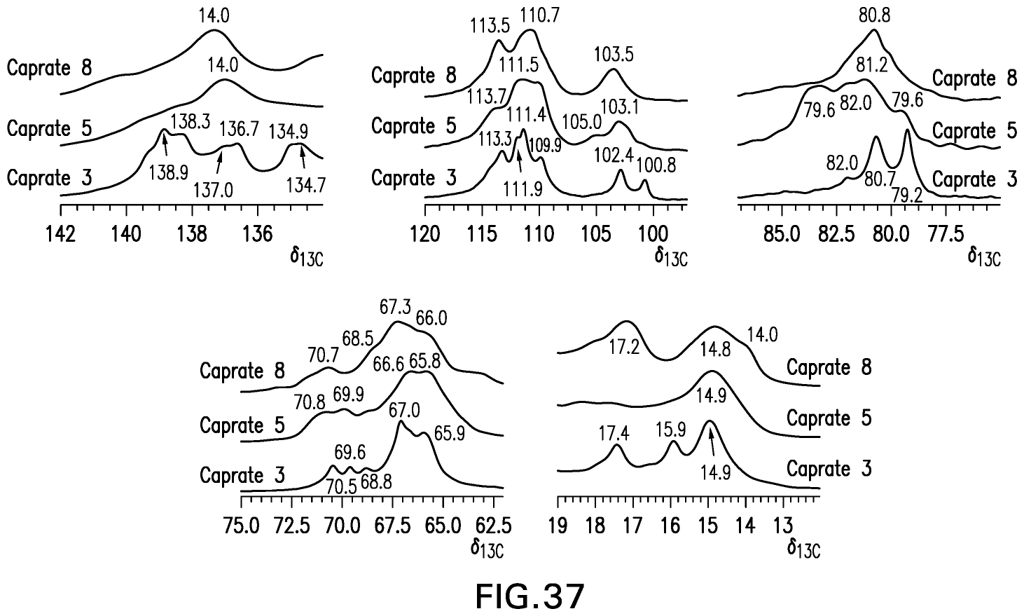

FIGS. 36A-C show the individual carbon-13 CPMAS spectra for Caprate 3, Caprate 5, and Caprate 8, respectively. The three caprate forms exhibit similar carbon-13 CPMAS spectra and, furthermore, small deviations in the respective spectra for a given form. Nevertheless, each form can be clearly identified by its carbon-13 CPMAS spectrum based on comparing specific spectral regions. FIG. 37 displays the spectral regions exhibiting the distinctive isotropic chemical shifts, and relative peak heights and shapes for each from.

What is claimed is:

1. A crystalline form of a compound of Formula I: 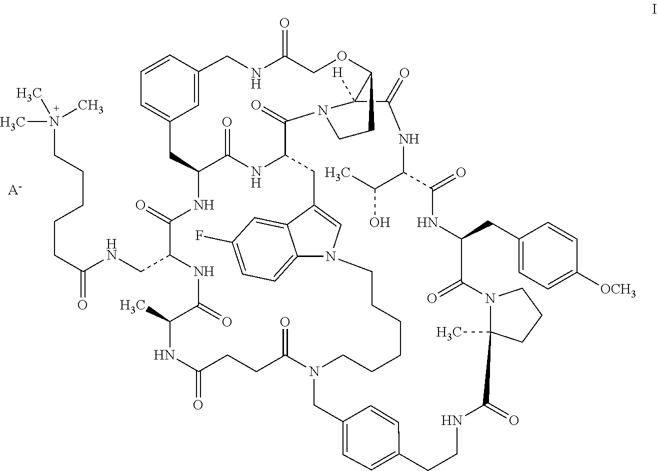
wherein A⁻ is a pharmaceutically acceptable anion.

2. The crystalline form of claim 1, wherein the crystalline form is selected from Acetate 2, Acetate 3, Acetate 4, Acetate 5, Acetate 6, Caprate 2, Caprate 3, Caprate 4, Caprate 5, Caprate 6, Caprate 7, Caprate 8, Caprate 9, Caprate 10, Caprate 11, Caprate 12, Caprate 13, Caprate 14, D-Lactate 1, D-Lactate 2, Succinate 1, Succinate 2, L-Tartrate 1, L-Tartrate 2, Sulfate 1, and Sulfate 2.

3. The crystalline form of claims 1-2, wherein the crystalline form is Acetate 2, wherein the crystalline form is characterized by an X-ray powder diffraction pattern having peaks expressed in degrees-2-theta at angles of (±0.2°) 4.92, 6.59, 9.82, and 17.91.

4. The crystalline form of claims 1-2, wherein the crystalline form is Acetate 3, wherein the crystalline form is characterized by an X-ray powder diffraction pattern having peaks expressed in degrees-2-theta at angles (±0.2°) of 4.48, 18.17, 18.79, and 19.27.

5. The crystalline form of claims 1-2, wherein the crystalline form is Acetate 4, wherein the crystalline form is characterized by an X-ray powder diffraction pattern having peaks expressed in degrees-2-theta at angles (±0.2°) of 8.36, 17.74, 20.29, and 21.35.

6. The crystalline form of claims 1-2, wherein the crystalline form is Acetate 5, wherein the crystalline form is characterized by an X-ray powder diffraction pattern having peaks expressed in degrees-2-theta at angles (±0.2°) of 5.02, 6.66, 9.89, and 19.84.

7. The crystalline form of claims 1-2, wherein the crystalline form is Acetate 6, wherein the crystalline form is characterized by an X-ray powder diffraction pattern having peaks expressed in degrees-2-theta at angles (±0.2°) of 7.79, 11.00, 16.24, and 18.89.

8. The crystalline form of claims 1-2, wherein the crystalline form is Caprate 2, wherein the crystalline form is characterized by an X-ray powder diffraction pattern having peaks expressed in degrees-2-theta at angles (±0.2°) of 4.85, 7.65, 17.16, 18.20, and 19.50.

9. The crystalline form of claims 1-2, wherein the crystalline form is Caprate 3, wherein the crystalline form is characterized by an X-ray powder diffraction pattern having peaks expressed in degrees-2-theta at angles (±0.2°) of 7.92, 15.40, 17.33, and 19.60.

10. The crystalline form of claims 1-2, wherein the crystalline form is Caprate 4, wherein the crystalline form is characterized by an X-ray powder diffraction pattern having peaks expressed in degrees-2-theta at angles (±0.2°) of 4.18, 6.14, 17.51, and 17.68.

11. The crystalline form of claims 1-2, wherein the crystalline form is Caprate 5, wherein the crystalline form is characterized by an X-ray powder diffraction pattern having peaks expressed in degrees-2-theta at angles (±0.2°) of 7.66, 16.18, 18.26, and 19.11.

12. The crystalline form of claims 1-2, wherein the crystalline form is Caprate 6, wherein the crystalline form is characterized by an X-ray powder diffraction pattern having peaks expressed in degrees-2-theta at angles (±0.2°) of 5.33, 6.97, 19.04, and 21.58.

13. The crystalline form of claims 1-2, wherein the crystalline form is Caprate 7, wherein the crystalline form is characterized by an X-ray powder diffraction pattern having peaks expressed in degrees-2-theta at angles (±0.2°) of 7.73, 17.14, 18.75, and 19.48.

14. The crystalline form of claims 1-2, wherein the crystalline form is Caprate 8, wherein the crystalline form is characterized by an X-ray powder diffraction pattern having peaks expressed in degrees-2-theta at angles (±0.2°) of 7.45, 17.97, 19.32, and 22.08.

15. The crystalline form of claims 1-2, wherein the crystalline form is Caprate 9, wherein the crystalline form is characterized by an X-ray powder diffraction pattern having peaks expressed in degrees-2-theta at angles (±0.2°) of 6.73, 11.95, 18.23, and 19.77.

16. The crystalline form of claims 1-2, wherein the crystalline form is Caprate 10, wherein the crystalline form is characterized by an X-ray powder diffraction pattern having peaks expressed in degrees-2-theta at angles (±0.2°) of about 3.50, 7.90, 16.21, and 18.23.

17. The crystalline form of claims 1-2, wherein the crystalline form is Caprate 11, wherein the crystalline form is characterized by an X-ray powder diffraction pattern having peaks expressed in degrees-2-theta at angles (±0.2°) of, 3.93, 4.90, and 7.68.

18. The crystalline form of claims 1-2, wherein the crystalline form is Caprate 12, wherein the crystalline form is characterized by an X-ray powder diffraction pattern having peaks expressed in degrees-2-theta at angles (±0.2°) of 6.80, 15.37, 18.22, and 20.63.

19. The crystalline form of claims 1-2, wherein the crystalline form is Caprate 13, wherein the crystalline form is characterized by an X-ray powder diffraction pattern having peaks expressed in degrees-2-theta at angles (±0.2°) of 5.02, 6.29, 7.12, and 20.25.

20. The crystalline form of claims 1-2, wherein the crystalline form is Caprate 14, wherein the crystalline form is characterized by an X-ray powder diffraction pattern having peaks expressed in degrees-2-theta radiation at angles (±0.2°) of 6.74, 18.16, 19.51, and 20.68.

21. A pharmaceutical composition comprising at least one crystalline form according to any one of claims 1-20 and a pharmaceutically acceptable carrier.

22. Use of the crystalline form according to any one of claims 1-20 as an active ingredient in a medicament for treating hypercholesterolemia in a subject.

23. Use of the crystalline form according to any one of claims 1-20 as an active ingredient in a medicament for treating hypercholesterolemia in a subject.

24. Use of the crystalline form according to any one of claims 1-20 as an active ingredient in a medicament for reducing LDL-C in a subject.

25. Use of the crystalline form according to any one of claims 1-20 as an active ingredient in a medicament for treating atherosclerotic cardiovascular disease in a subject.

26. A method of treating hypercholesterolemia, comprising administering to a patient in need thereof a therapeutically effective amount of a crystalline form of any one of claims 1-20.

27. A crystalline form of a compound of Formula I: 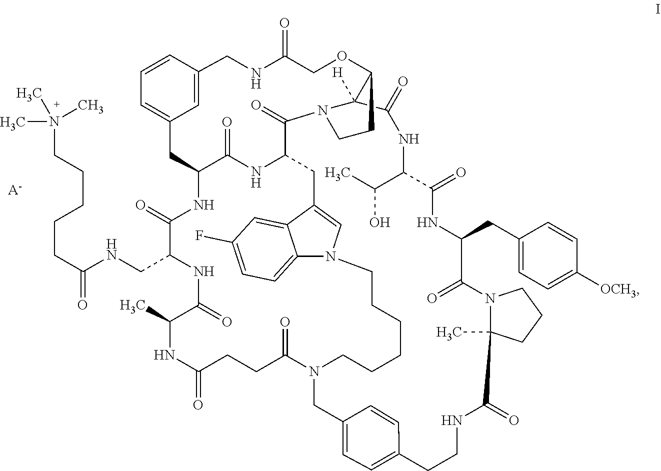
wherein A⁻ is a pharmaceutically acceptable anion;
formed by a process comprising adding an alcohol to a starting material,
wherein the starting material is selected from Compound 1, Compound 2, Compound 3, Compound 4, Compound 5, and Compound 6.

28. The crystalline form, according to claim 27, of a compound of Formula I: 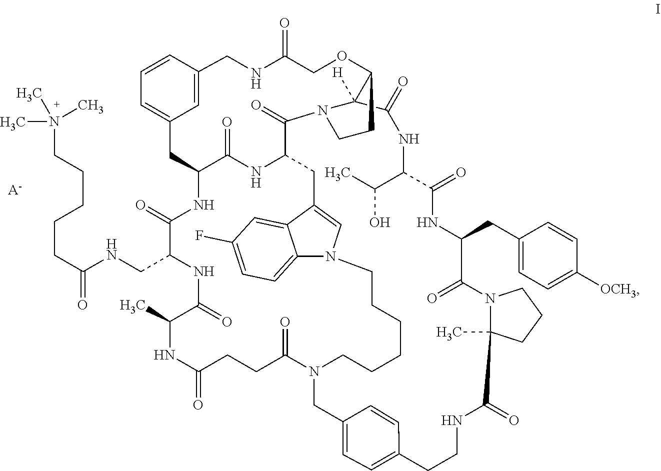
wherein A⁻ is caprate;
formed by a process comprising adding an alcohol to a form of Compound 2.

29. The crystalline form of any one of claims 27-28, wherein the crystalline form is selected from Caprate 2, Caprate 3, Caprate 4, Caprate 5, Caprate 6, Caprate 7, Caprate 8, Caprate 9, Caprate 10, Caprate 11, Caprate 12, Caprate 13, and Caprate 14.

30. The crystalline form of any one of claims 27-29, wherein the alcohol is selected from: ethanol, propanol, or butanol.

31. The crystalline form of any one of claim 27-30, wherein the process comprises adding a solvent system to Compound 2, wherein the solvent system comprises MTBE, water, and the alcohol.

32. The crystalline form of claim 31, wherein the solvent system is comprised of MTBE, approximately 30-40% weight 1-propanol and 0.5-5% water.

33. The crystalline form of claim 32, wherein the crystalline form is Caprate 3 and the process further comprises filtering the solvent system resulting in the formation of a wet cake and drying the wet cake.

34. A method of inhibiting PCSK9 activity in a subject in need of such treatment comprising orally administering to the subject an amount of a crystalline form, according to claim 1, of a compound of Formula I: 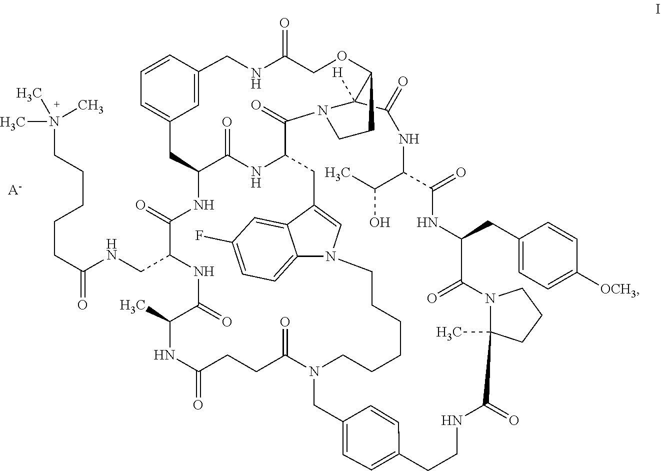
wherein A⁻ is a pharmaceutically acceptable anion, and wherein the amount is from about 5 mg to about 300 mg of the crystalline form of the compound of Formula I.

35. A pharmaceutical composition comprising a crystalline form, according to claim 1, of a compound of Formula I: 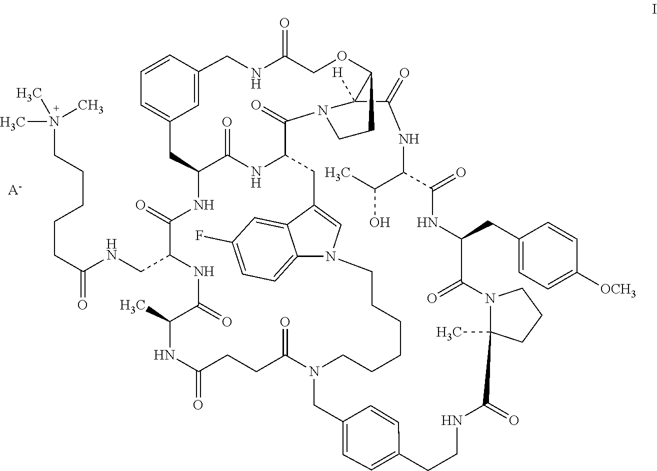
wherein A⁻ is a pharmaceutically acceptable anion, and wherein the amount is from about 5 mg to about 300 mg of the crystalline form of the compound of Formula I, and a permeation enhancer.

36. The pharmaceutical composition of claim 35, wherein the permeation enhancer is sodium caprate.

37. The pharmaceutical composition of any of claims 35-36, wherein the crystalline form of the compound of Formula I is Caprate 3.
